(19) United States
(12) Patent Application Publication
(10) Pub. No.: US 2012/0286251 A1
(43) Pub. Date: Nov. 15, 2012

(54) NOVEL COMPOUND AND ORGANIC LIGHT-EMITTING DEVICE INCLUDING THE SAME

(76) Inventors: Soo-Jin Park, Yongin-city (KR); Tsuyoshi Naijo, Yongin-city (KR); Keon-Ha Choi, Yongin-city (KR); Ji-Hye Shim, Yongin-city (KR); Jong-Tae Je, Cheongwon-gun (KR); Jeong-Soo Kim, Cheongwon-gun (KR); Hyun-Jung Kwon, Cheongwon-gun (KR); Yoo-Na Shin, Cheongwon-gun (KR)

(21) Appl. No.: 13/451,424

(22) Filed: Apr. 19, 2012

(30) Foreign Application Priority Data
May 9, 2011 (KR) 10-2011-0043650
Dec. 1, 2011 (KR) 10-2011-0127858

(51) Int. Cl. H01L 51/50; C07C 211/54; C07C 23/18; C07C 13/70; C07D 211/80

(52) U.S. Cl. 257/40; 585/27; 585/26; 546/285; 570/183; 564/427; 257/E51.019

(57) ABSTRACT

Embodiments of the present invention are directed to a compound represented by Formula 1, and an organic light-emitting device including an organic film that includes the compound of Formula 1:

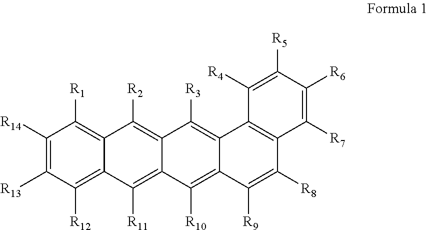

### CROSS-REFERENCE TO RELATED PATENT APPLICATION

This application claims priority to and the benefit of Korean Patent Application Nos. 10-2011-0043650 filed on May 9, 2011 in the Korean Intellectual Property Office, and 10-2011-0127858 filed on Dec. 1, 2011 in the Korean Intellectual Property Office, the entire contents of each of which are incorporated herein by reference.

### BACKGROUND

1. Technical Field

The present invention relates to a compound represented by Formula 1, and an organic light-emitting device including the same.

2. Description of Related Art

Recently, large display devices have become more common, and demand for flat display devices that occupy relatively small spaces is increasing. A liquid crystal display device is a representative flat display device that is lightweight compared to existing cathode ray tubes (CRTs). However, the liquid crystal display device has a limited viewing angle and necessarily requires back light. Another example of a flat display device is an organic light-emitting diode (OLED), which is a self-emission display device, and has a wide viewing angle, is lightweight and has a simplified structure compared to the liquid crystal display device, and has a short response speed. Also, OLEDs are expected to be applied in full-color displays or illumination devices in the future.

In general, organic light emission refers to the conversion of electrical energy into light energy using an organic material.

OLEDs operate based on the organic light emission phenomenon, and each OLED typically includes a cathode, an anode, and an organic material layer between the cathode and the anode. In this regard, the organic material layer may have a multi-layered structure including a plurality of layers formed of different materials to increase the efficiency and stability of a formed OLED. For example, the organic material layer may include a hole injection layer, a hole transport layer, an emission layer, an electron transport layer, an electron injection layer, etc. In an OLED having such a structure, if a voltage is applied between the cathode and the anode, holes are injected into the organic material layer through the cathode, and electrons are injected into the organic material layer through the anode, and when the holes and the electrons are recombined, excitons are formed, and when the excitons return to the ground state, light is generated. OLEDs are a type of self-emission device and have high brightness, high efficiency, low driving voltage, wide viewing angles, high contrast ratios, and high-speed response properties.

### SUMMARY

Embodiments of the present invention provide a novel compound having low driving voltage and high luminescent efficiency.

Embodiments of the present invention provide an organic light-emitting device including the novel compound.

Embodiments of the present invention provide a flat panel display device including the organic light-emitting device.

According to embodiments of the present invention, a compound is represented by Formula 1 below:

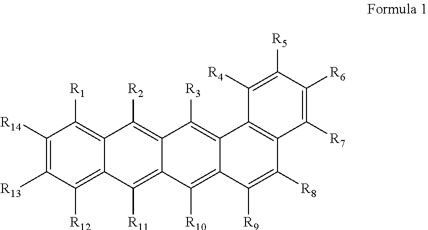

In Formula 1, R₁ to R₁₄ may independently be a hydrogen atom, a deuterium atom, a substituted or unsubstituted C1 to C60 alkyl group, a substituted or unsubstituted C2 to C60 alkenyl group, a substituted or unsubstituted C2 to C60 alkynyl group, a substituted or unsubstituted C3 to C60 cycloalkyl group, a substituted or unsubstituted C1 to C60 alkoxy group, a substituted or unsubstituted C5 to C60 aryloxy group, a substituted or unsubstituted C5 to C60 arylthio group, a substituted or unsubstituted C5 to C60 aryl group, an amino group substituted with a C5 to C60 aryl group or a C3 to C60 heteroaryl group, a substituted or unsubstituted C3 to C60 heteroaryl group, a substituted or unsubstituted C6 to C60 fused polycyclic ring, a halogen atom, a cyano group, a nitro group, a hydroxyl group, or a carboxyl group.

According to an embodiment of the present invention, in Formula 1, each of R₁ to R₁₄ may independently be a hydrogen atom, a deuterium atom, a cyano group, a halogen atom, a substituted or unsubstituted C1 to C30 alkyl group, a substituted or unsubstituted C5 to C30 aryl group, a substituted or unsubstituted C3 to C30 heteroaryl group, an amino group substituted with a C5 to C30 aryl group or a C3 to C30 heteroaryl group, or a substituted or unsubstituted C6 to C30 fused polycyclic ring.

According to another embodiment of the present invention, in Formula 1, each of R₁ to R₁₄ may independently be a hydrogen atom, a deuterium atom, a halogen atom, a cyano group, a substituted or unsubstituted C1 to C20 alkyl group, or a compound represented by one of Formulae 2a to 2g below:

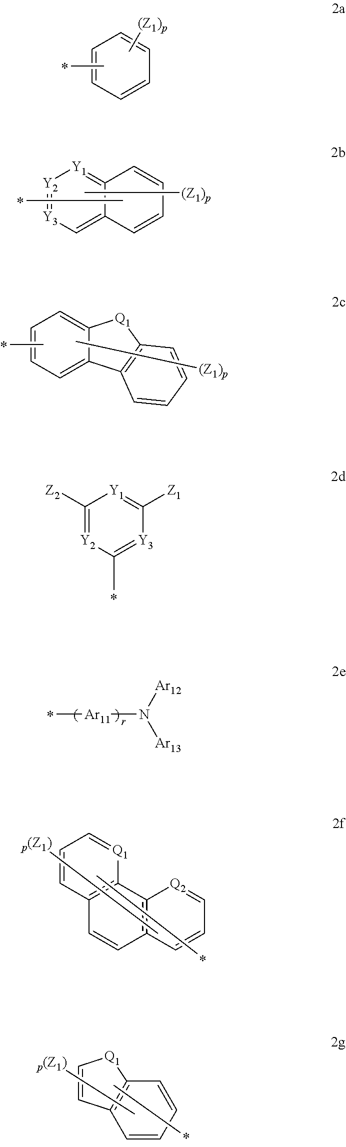

In Formulae 2a to 2g, each of Q₁ and Q₂ are linkers that can be represented by —C(R₂₀)(R₂₁)—, —N(R₂₀)—, —S—, or —O—. Each of Y₁, Y₂, and Y₃ is a linker that may independently be represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. In some embodiments, adjacent Ar₁₂ and Ar₁₃ groups, or adjacent R₂₀ and R₂₁ groups may be fused with each other to form a ring, or may be linked to each other via a single bond. Ar₁₁ may be a substituted or unsubstituted C1 to C20 alkylene group, a substituted or unsubstituted C5 to C20 arylene group, or a substituted or unsubstituted C3 to C20 heteroarylene group. Also, p is an integer of 1 to 12, r is an integer of 0 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom.

According to another embodiment of the present invention, in Formula 1, R₂ and R₁₁ may be identical to each other.

According to another embodiment of the present invention, each of R₁ to R₁₄ may independently be a hydrogen atom, a deuterium atom, a halogen atom, a cyano group, a substituted or unsubstituted C1 to C20 alkyl group, a substituted or unsubstituted C3 to C30 heteroaryl group, a substituted or unsubstituted C6 to C30 fused polycyclic ring, or a compound represented by one of Formulae 3a to 3f below:

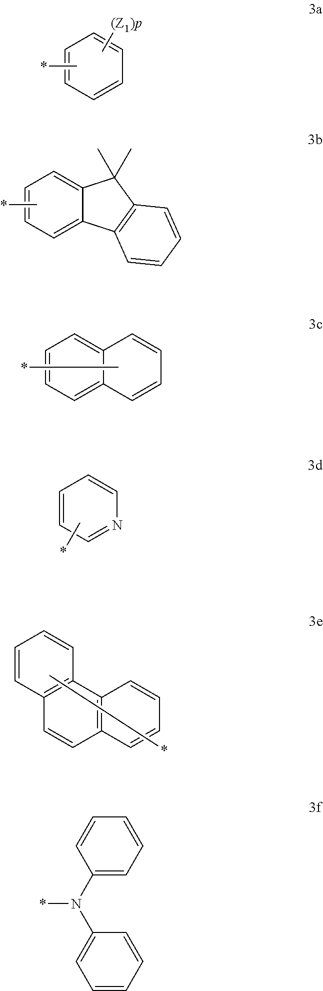

In Formulae 3a to 3f, Z₁ may be a hydrogen atom, a deuterium atom, or a substituted or unsubstituted C5 to C20 aryl group. Also, p is an integer of 1 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 2a to 2g below:

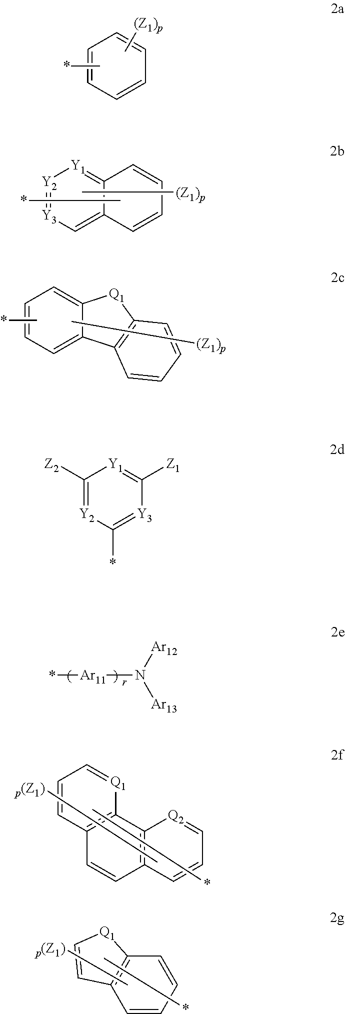

In Formulae 2a to 2g, each of Q₁ and Q₂ is a linker that may be independently represented by —C(R₂₀)(R₂₁)—, —N(R₂₀)—, —S—, or —O—. Each of Y₁, Y₂, and Y₃ is a linker that may be independently represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. In some embodiments, adjacent Ar₁₂ and Ar₁₃ groups, or adjacent R₂₀, and R₂₁ groups, may be fused with each other to form a ring, or linked to each other via a single bond. Ar₁₁ may be a substituted or unsubstituted C1 to C20 alkylene group, a substituted or unsubstituted C5 to C20 arylene group, or a substituted or unsubstituted C3 to C20 heteroarylene group. Also, p is an integer of 1 to 12, r is an integer of 0 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 3a to 3f below:

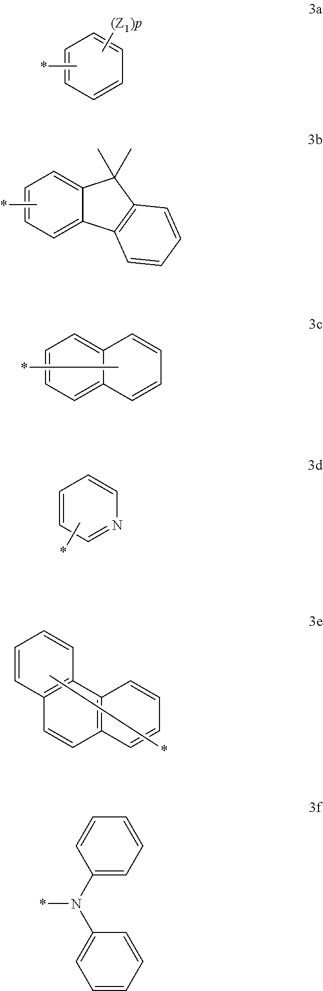

In Formulae 3a to 3f, Z₁ may be a hydrogen atom, a deuterium atom, or a substituted or unsubstituted C5 to C20 aryl group. Also, p is an integer of 1 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 2a to 2g below, and R₂ and R₁₁ may be identical to each other:

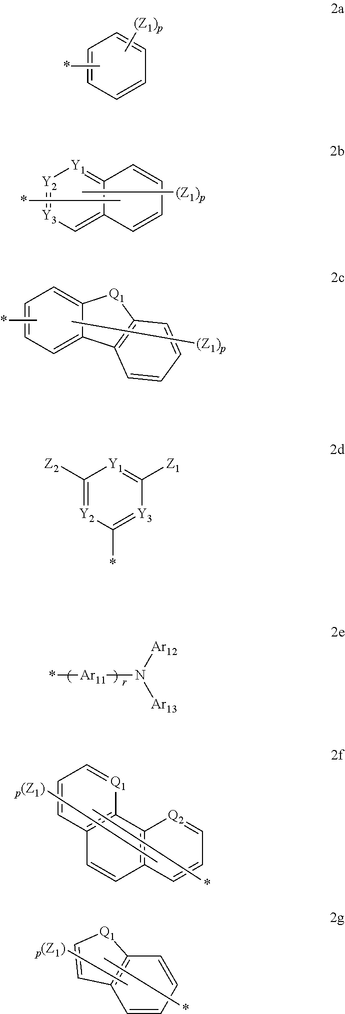

In Formulae 2a to 2g, each of Q₁ and Q₂ may be a linker independently represented by —C(R₂₀)(R₂₁)—, —N(R₂₀)—, —S—, or —O—. Each of Y₁, Y₂, and Y₃ may independently be a linker represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. Adjacent Ar₁₂ and Ar₁₃ groups, or adjacent R₂₀ and R₂₁ groups, may be fused with each other to form a ring, or linked to each other via a single bond. Ar₁₁ may be a substituted or unsubstituted C1 to C20 alkylene group, a substituted or unsubstituted C5 to C20 arylene group, or a substituted or unsubstituted C3 to C20 heteroarylene group. Also, p is an integer of 1 to 12, r is an integer of 0 to 5, and * indicates a binding site.

According to another embodiment of the present invention, the compound of Formula 1 may be one of Compounds 10, 24, 28, 29, 83, 144, 231 and 330, below:

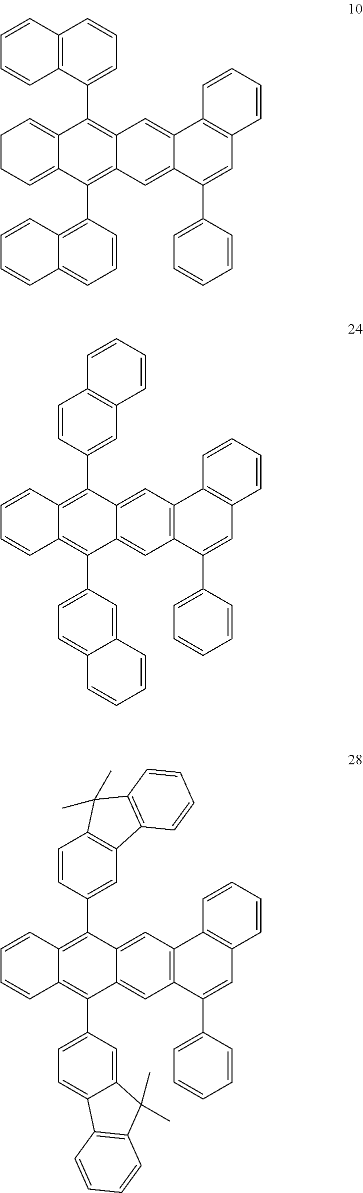

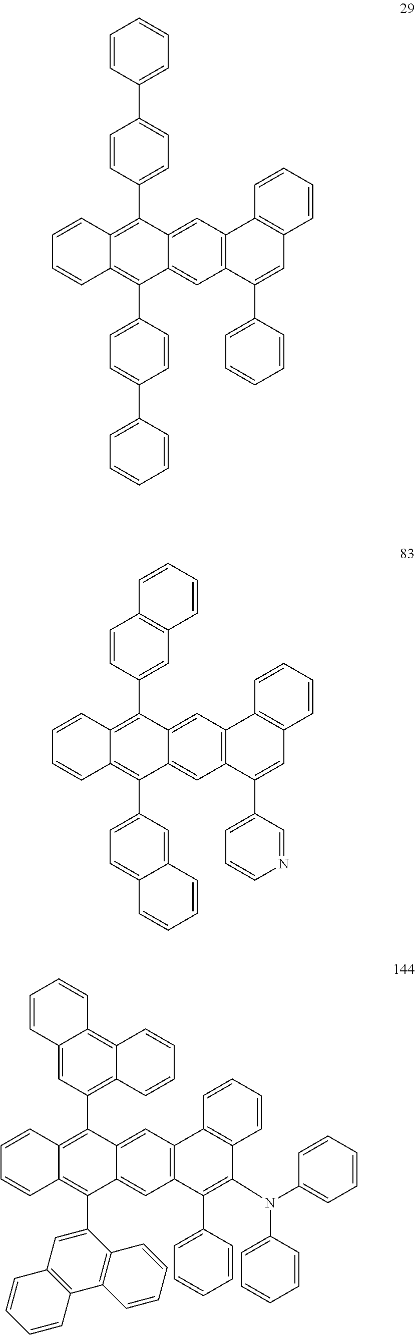

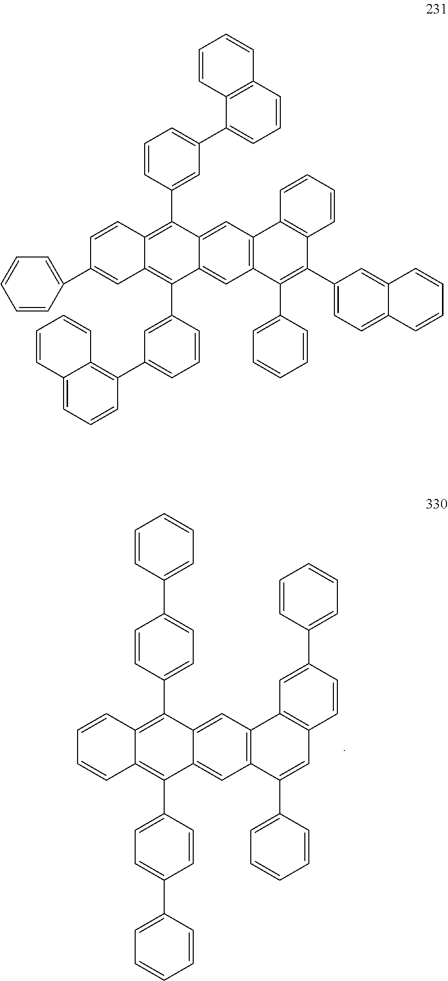

According to other embodiments of the present invention, an organic light-emitting device includes: a first electrode; a second electrode; and an organic layer between the first electrode and the second electrode, where the organic layer includes the compound represented by Formula 1.

According to an embodiment of the present invention, the organic layer may be a hole injection layer, a hole transport layer, a functional layer having hole injection and hole transport capabilities, an electron injection layer, an electron transport layer, or a functional layer having electron transport and electron injection capabilities.

According to other embodiments of the present invention, the organic light-emitting device may include an electron transport layer that includes an electron transporting organic material and a metal-containing material.

According to another embodiment of the present invention, the organic layer may be an emission layer, and the compound represented by Formula 1 may be used as a host for a fluorescence or phosphorescence device.

According to another embodiment of the present invention, the organic light-emitting device may include an emission layer, a hole transport layer, and an electron transport layer, and the emission layer may further include an anthracene compound, arylamine compound, or a styryl compound.

According to another embodiment of the present invention, the organic light-emitting device may include an emission layer, a hole transport layer, and an electron transport layer, and the emission layer may include a red layer, a green layer, a blue layer, and a white layer, and any one of these layers may further include a phosphorescent compound.

The organic layer may be a red emission layer.

The organic layer may be a red emission layer, and the compound of Formula 1 may be used as a red host.

According to another embodiment of the present invention, the organic layer may be formed by a wet process using the compound of claim 1.

According to another embodiment of the present invention, a flat panel display device includes the organic light-emitting device, and the first electrode of the organic light-emitting device is electrically connected to a source electrode or a drain electrode of a thin film transistor.

### BRIEF DESCRIPTION OF THE DRAWINGS

The above, and other features, and advantages of the present invention will become more apparent by reference to the following detailed description when considered in conjunction with the following drawing, in which:

FIG. 1 is a schematic sectional view of an organic light-emitting device according to an embodiment of the present invention.

### DETAILED DESCRIPTION OF THE INVENTION

Expressions such as “at least one of” used herein, when preceding a list of elements, modify the entire list of elements, and do not modify the individual elements of the list.

A material used in an organic material layer that is included in an organic light-emitting device can be categorized as a light-emitting material or a charge transport material according to its function (for example, a hole injection material, a hole transport material, an electron transport material, an electron injection material, etc.). The light-emitting material can be categorized as a polymer material or a low molecular weight material, according to its molecular weight, and can also be categorized as a fluorescent material (derived from a singlet excited state of an electron), or a phosphorescent material (derived from a triplet excited state of an electron), according to the emission mechanism. Also, the light-emitting material can be categorized as a blue light-emitting material, a green light-emitting material, a red light-emitting material, a yellow light-emitting material, or an orange light-emitting material, according to the emission color, and the yellow and orange light-emitting materials are used to obtain more natural color.

Also, if only one material is used as the light-emitting material, the maximum light emission wavelength may be shifted toward a longer wavelength due to intermolecular interaction, and color purity or light emission may be decreased, and thus, a formed device may have lower efficiency. Accordingly, to increase color purity and to increase light emission efficiency through energy transition, a host-dopant system may be used as the light-emitting material.

This mechanism is as follows: if a dopant that has a narrower energy band interval than a host used in forming an emission layer is included in a small amount in an emission layer, excitons generated from the emission layer may be transported by the dopant and thus, light emission efficiency may be improved. In this case, the wavelength range of the host is moved toward the wavelength range of the dopant. Accordingly, the wavelength of light may be controlled by the dopant.

To manufacture an organic light-emitting device having such improved characteristics, a material included in an organic material layer (for example, a hole injection material, a hole transport material, a light-emitting material, an electron transport material, an electron injection material, etc.) should be stable and efficient. However, up to now, a stable and efficient material for an organic material layer for use in an organic light-emitting device has not been satisfactorily developed. Accordingly, demand for developing new materials continues in the art.

According to embodiments of the present invention, a compound is represented by Formula 1 below:

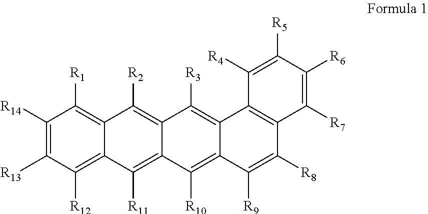

In Formula 1, each of R₁ to R₁₄ may independently be a hydrogen atom, a deuterium atom, a substituted or unsubstituted C1 to C60 alkyl group, a substituted or unsubstituted C2 to C60 alkenyl group, a substituted or unsubstituted C2 to C60 alkynyl group, a substituted or unsubstituted C3 to C60 cycloalkyl group, a substituted or unsubstituted C1 to C60 alkoxy group, a substituted or unsubstituted C5 to C60 aryloxy group, a substituted or unsubstituted C5 to C60 arylthio group, a substituted or unsubstituted C5 to C60 aryl group, an amino group substituted with a C5 to C60 aryl group or a C3 to C60 heteroaryl group, a substituted or unsubstituted C3 to C60 heteroaryl group, a substituted or unsubstituted C6 to C60 fused polycyclic ring, a halogen atom, a cyano group, a nitro group, a hydroxyl group, or a carboxyl group.

The compound of Formula 1 may be used as a light-emitting material for an organic light-emitting device. The compound of Formula 1 is stable and has good luminescence characteristics. An organic light-emitting device manufactured using the compound of Formula 1 is driven at low voltage and has improved luminescent efficiency.

Substituents included in the compound of Formula 1 are described in detail below.

According to an embodiment of the present invention, in Formula 1, each of R₁ to R₁₄ may independently be a hydrogen atom, a deuterium atom, a cyano group, a halogen atom, a substituted or unsubstituted C1 to C30 alkyl group, a substituted or unsubstituted C5 to C30 aryl group, a substituted or unsubstituted C3 to C30 heteroaryl group, an amino group substituted with a C5 to C30 aryl group or a C3 to C30 heteroaryl group, or a substituted or unsubstituted C6 to C30 fused polycyclic ring.

According to another embodiment of the present invention, in Formula 1, each of R₁ to R₁₄ may independently be a hydrogen atom, a deuterium atom, a halogen atom, a cyano group, a substituted or unsubstituted C1 to C20 alkyl group, or a compound represented by one of Formulae 2a to 2g:

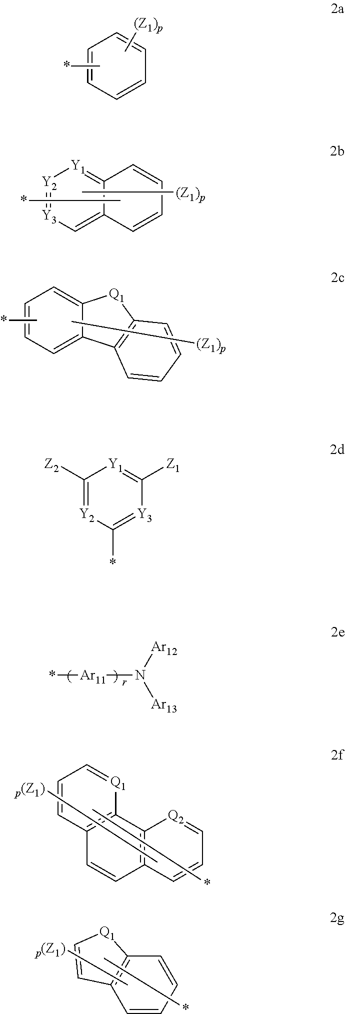

In Formulae 2a to 2g, each of Q₁ and Q₂ may independently be a linker represented by —C(R₂₀)(R₂₁)—, —N(R₂₀)—, —S—, or —O—. Each of Y₁, Y₂, and Y₃ may independently be a linker represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. Adjacent Ar₁₂ and Ar₁₃ groups, or adjacent R₂₀ and R₂₁ groups, may optionally be fused with each other to form a ring, or linked to each other via a single bond. Ar₁₁ may be a substituted or unsubstituted C1 to C20 alkylene group, a substituted or unsubstituted C5 to C20 arylene group, or a substituted or unsubstituted C3 to C20 heteroarylene group. Also, p may be an integer of 1 to 12, r may be an integer of 0 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom.

According to another embodiment of the present invention, in Formula 1, R₂ and R₁₁ may be identical to each other.

According to another embodiment of the present invention, each of R₁ to R₁₄ may independently be a hydrogen atom, a deuterium atom, a halogen atom, a cyano group, a substituted or unsubstituted C1 to C20 alkyl group, a substituted or unsubstituted C3 to C30 heteroaryl group, a substituted or unsubstituted C6 to C30 fused polycyclic ring, or a compound represented by one of Formulae 3a to 3f below:

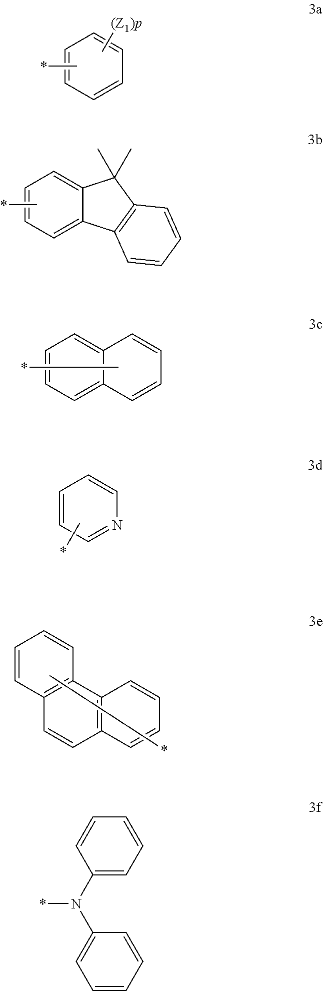

In Formulae 3a to 3f, Z₁ may be a hydrogen atom, a deuterium atom, or a substituted or unsubstituted C5 to C20 aryl group. Also, p may be an integer of 1 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 2a to 2g below:

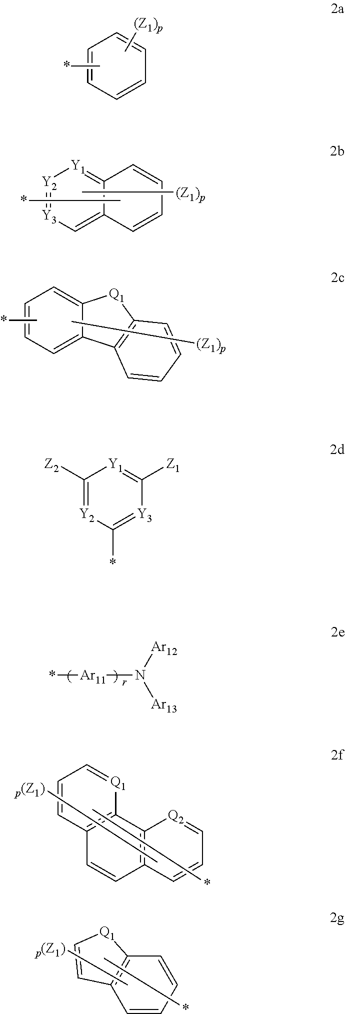

In Formulae 2a to 2g, each of Q₁ and Q₂ may independently be a linker represented by —C(R₂₀)(R₂₁)—, —N(R₂₀)—, —S—, or —O—. Each of Y₁, Y₂, and Y₃ may independently be a linker represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. Adjacent Ar₁₂ and Ar₁₃ groups, or adjacent R₂₀ and R₂₁ groups, may optionally be fused with each other to form a ring, or may be linked to each other via a single bond. Ar₁₁ may be a substituted or unsubstituted C1 to C20 alkylene group, a substituted or unsubstituted C5 to C20 arylene group, or a substituted or unsubstituted C3 to C20 heteroarylene group. Also, p may be an integer of 1 to 12, r may be an integer of 0 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 3a to 3f below:

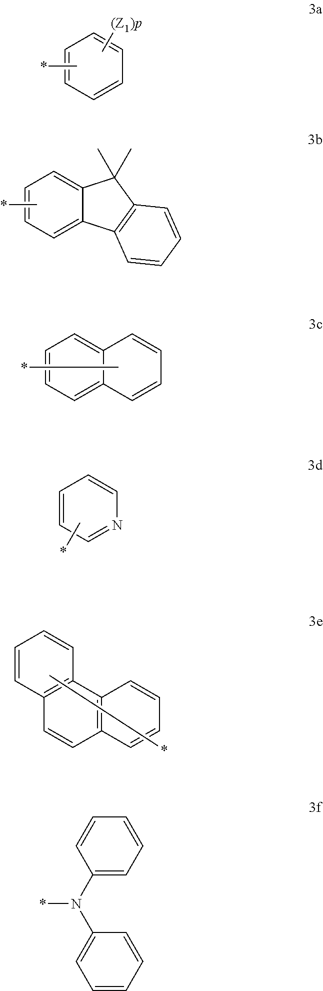

In Formulae 3a to 3f, Z₁ may be a hydrogen atom, a deuterium atom, or a substituted or unsubstituted C5 to C20 aryl group. Also, p may be an integer of 1 to 5, and * indicates a binding site.

According to another embodiment of the present invention, in Formula 1, each of R₁, R₃, R₄, R₇, R₁₀, and R₁₂ to R₁₄ may independently be a hydrogen atom or a deuterium atom, and each of R₂, R₅, R₆, R₈, R₉, and R₁₁ may independently be a hydrogen atom, a deuterium atom, a halogen atom, a cyano group, a substituted or unsubstituted C1 to C20 alkyl group, or a compound represented by one of Formulae 2a to 2g below, and R₂, and R₁₁ may be identical to each other:

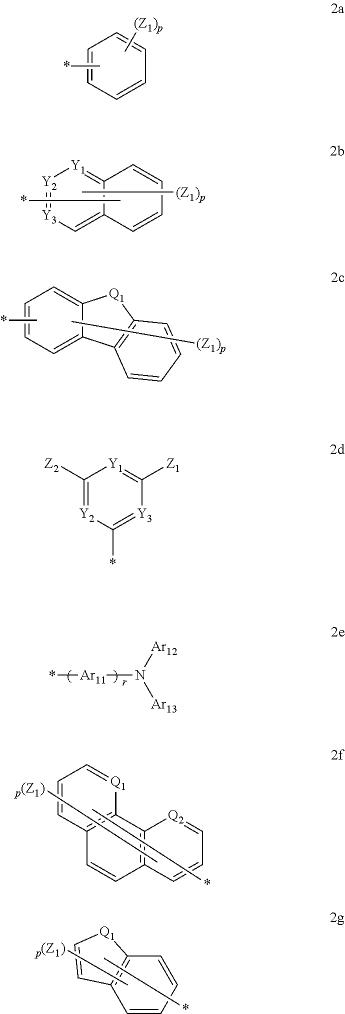

In Formulae 2a to 2g, each of Q₁ and Q₂ may independently be a linker represented by —C(R₂₀)(R₂₁)—, —N(R₂₀)—, —S—, or —O—. Each of Y₁, Y₂, and Y₃ may independently be a linker represented by —N═ or —C(R₂₂)═. Each of Z₁, Z₂, Ar₁₂, Ar₁₃, R₂₀, R₂₁, and R₂₂ may independently be a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group. Adjacent Ar₁₂ and Ar₁₃ groups, or adjacent R₂₀ and R₂₁ groups, may optionally be fused with each other to form a ring, or may be linked to each other via a single bond (for example, in Formula 2c, if Q₁ is —C(R₂₀)(R₂₁)—, R₂₀ and R₂₁ are phenyl groups, and R₂₀ and R₂₁ are linked to each other via a single bond, a Spiro fluorene group is formed). Ar₁₁ may be a substituted or unsubstituted C1 to C20 alkylene group, a substituted or unsubstituted C5 to C20 arylene group, or a substituted or unsubstituted C3 to C20 heteroarylene group. Also, p may be an integer of 1 to 12, r may be an integer of 0 to 5, and * indicates a binding site.

Hereinafter, definitions of some of the groups used in the formulae above are presented below (the number of carbon atoms in the substituents is non-limiting, and does not limit the characteristics of the respective substituents).

The unsubstituted C1 to C60 alkyl group, as used herein, refers to a linear or branched group, and non-limiting examples thereof include methyl, ethyl, propyl, isobutyl, sec-butyl, pentyl, iso-amyl, hexyl, heptyl, octyl, nonanyl, dodecyl, etc. In the substituted C1 to C60 alkyl group, at least one hydrogen atom of the alkyl group may be substituted with a deuterium atom, a halogen atom, a hydroxyl group, a nitro group, a cyano group, an amino group, an amidino group, a hydrazine group, a hydrazone group, a carboxylic acid group or salt thereof, a sulfonic acid group or salt thereof, a phosphoric acid group or salt thereof, a C1 to C10 alkyl group, a C1 to C10 alkoxy group, a C2 to C10 alkenyl group, a C2 to C10 alkynyl group, a C6 to 16 aryl group, or a C4 to 16 heteroaryl group.

The unsubstituted C2 to C60 alkenyl group, as used herein, refers to a group (such as any of the unsubstituted alkyl groups described above) that has one or more carbon-to-carbon double bonds. Non-limiting examples thereof include an ethenyl group, a propenyl group, a butenyl group, etc. In the substituted C2 to C60 alkenyl group, at least one hydrogen atom of the unsubstituted alkenyl group may be substituted with any one of the substituents described above with respect to the substituted alkyl group.

The unsubstituted C2 to C60 alkynyl group used herein refers to a group (such as any of the unsubstituted alkyl groups defined above) that has one or more carbon-to-carbon triple bonds. Non-limiting examples thereof include acetylenyl, propylenyl, phenylacetylenyl, naphthylacetylenyl, isopropylacetylenyl, t-butylacetylenyl, diphenylacetylenyl, etc. In the substituted C2 to C60 alkynyl group, at least one hydrogen atom of the alkynyl group may be substituted with any one of the substituents described above with respect to the substituted alkyl group.

The unsubstituted C3 to 60 cycloalkyl group, as used herein, refers to a C3 to C60 cyclic alkyl group. In the substituted C3 to C60 cycloalkyl group, at least one hydrogen atom of the cycloalkyl group may be substituted with any one of the substituents described above with respect to the substituted C1 to C60 alkyl group.

The unsubstituted C1 to C60 alkoxy group, as used herein, refers to a group represented by -OA (where A is an unsubstituted C1 to C60 alkyl group (described above)). Non-limiting examples thereof include methoxy, ethoxy, propoxy, isopropyloxy, butoxy, pentoxy, etc. In the substituted C1 to C60 alkoxy group, at least one hydrogen atom of the alkoxy group may be substituted with any one of the substituents described above with respect to the substituted alkyl group.

The unsubstituted C5 to 60 aryl group, as used herein, refers to a carbocyclic aromatic system having one or more rings. If two or more rings are included, the rings may be fused with each other, or may be linked to each other via a single bond. The term ‘aryl’ includes an aromatic system, such as phenyl, naphthyl, or anthracenyl. Also, in the substituted C5 to C60 aryl group, at least one hydrogen atom of the aryl group may be substituted with any one of the substituents described above with respect to the C1 to C60 alkyl group.

Non-limiting examples of the substituted or unsubstituted C5 to 60 aryl group include a phenyl group, a C1 to C10 alkylphenyl group (for example, ethylphenyl group), a halophenyl group (for example, o-, m-, and p-fluorophenyl groups, a dichlorophenyl group), a cyanophenyl group, a dicyanophenyl group, a trifluoromethoxyphenyl group, a biphenyl group, a halobiphenyl group, a cyanobiphenyl group, a C1 to C10 alkylbiphenyl group, a C1 to C10 alkoxybiphenyl group, o-, m-, and p-tolyl groups, o-, m-, and p-cumenyl groups, a mesityl group, a phenoxyphenyl group, a (α,α-dimethylbenzene)phenyl group, a (N,N′-dimethyl)aminophenyl group, a (N,N′-diphenyl)aminophenyl group, a pentalenyl group, an indenyl group, a naphthyl group, a halonaphthyl group (for example, fluoronaphthyl group), a C1 to C10 alkylnaphthyl group (for example, methylnaphthyl group), a C1 to C10 alkoxynaphthyl group (for example, a methoxynaphthyl group), a cyanonaphthyl group, an anthracenyl group, an azulenyl group, a heptalenyl group, an acenaphthylenyl group, a phenalenyl group, a fluorenyl group, an anthraquinolyl group, a methylanthryl group, a phenanthryl group, a triphenylenyl group, a pyrenyl group, a chrysenyl group, an ethyl-chrysenyl group, a picenyl group, a perylenyl group, a chloroperylenyl group, a pentaphenyl group, a pentacenyl group, a tetraphenylenyl group, a hexaphenyl group, a hexacenyl group, a rubicenyl group, a coroneryl group, a trinaphthylenyl group, a heptaphenyl group, a heptacenyl group, a pyranthrenyl group, an ovalenyl group, etc.

The unsubstituted C4 to 60 heteroaryl group, as used herein, includes at least one ring having one or more heteroatoms selected from the group consisting of nitrogen (N), oxygen (O), phosphorous (P), and sulfur (S). If two or more rings are included, the rings may be fused with each other, or may be linked to each other via a single bond. Non-limiting examples of the unsubstituted C4 to 60 heteroaryl group include a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolyl group, an isoquinolyl group, a dibenzothiophenyl group etc. Also, in the substituted C4 to C60 heteroaryl group, at least one hydrogen atom of the heteroaryl group may be substituted with any one of the substituents described above with respect to the C1 to C60 alkyl group.

The unsubstituted C5 to 60 aryloxy group, as used herein, refers to a group represented by -OA₁ where A₁ may be a C5 to 60 aryl group. An example of the aryloxy group is a phenoxy group. In the substituted C5 to C60 aryloxy group, at least one hydrogen atom of the aryloxy group may be substituted with any one of the substituents described above with respect to the C1 to C60 alkyl group.

The unsubstituted C5 to 60 arylthio group, as used herein, refers to a group represented by —SA₁ where A₁ may be a C5 to 60 aryl group. Non-limiting examples of the arylthio group include a benzylthio group and a naphthylthio group. In the substituted C5 to C60 arylthio group, at least one hydrogen atom of the arylthio group may be substituted with any one of the substituents described above with respect to the C1 to C60 alkyl group.

The unsubstituted C6 to 60 fused polycyclic ring, as used herein, refers to a substituent including at least two rings that are fused to each other. The at least two rings may include at least one aromatic ring and/or at least one non-aromatic ring. The unsubstituted C6 to 60 fused polycyclic ring is distinguished from the aryl or heteroaryl group in that the unsubstituted C6 to 60 fused polycyclic ring does not have an entirely aromatic property. In the substituted C6 to C60 fused polycyclic ring, at least one hydrogen atom of the fused polycyclic ring may be substituted with any one of the substituents discussed above with respect to the C1 to C60 alkyl group.

A non-limiting example of the substituted fused polycyclic ring is a compound having the following structure:

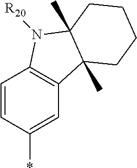

In the above structure, R₂₀ and * are the same as defined above.

Non-limiting examples of compounds represented by Formula 1 include Compounds 10 to 333 illustrated below.

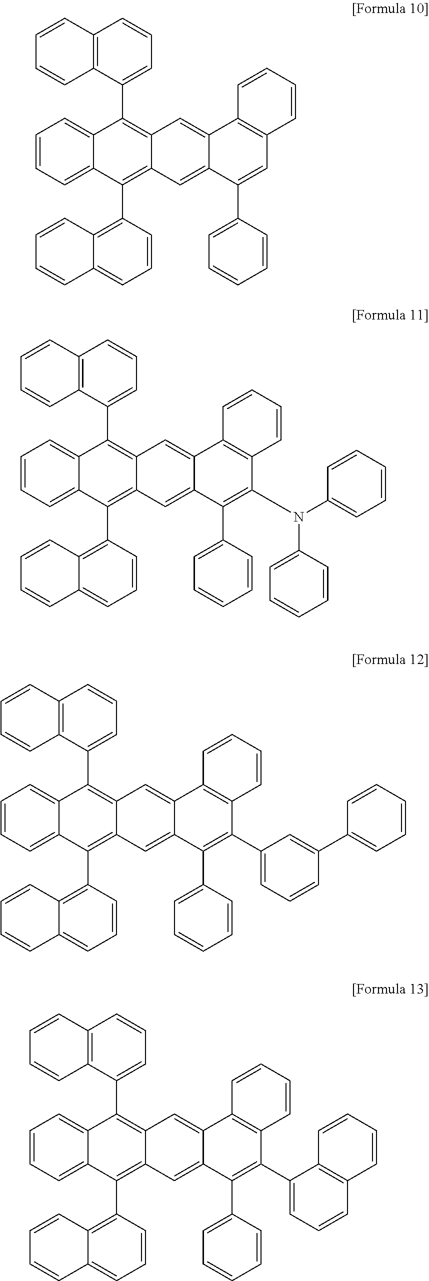

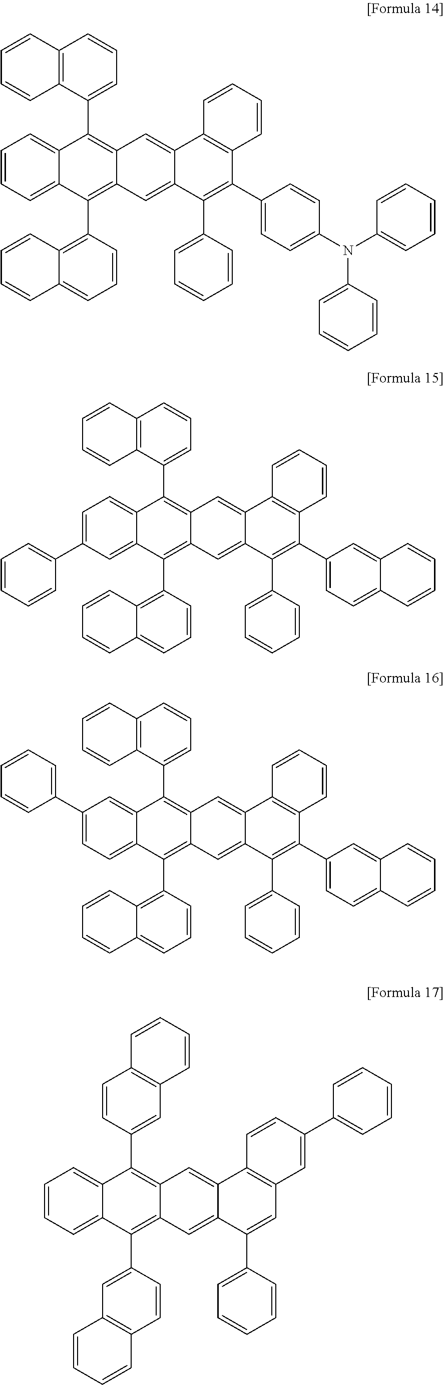

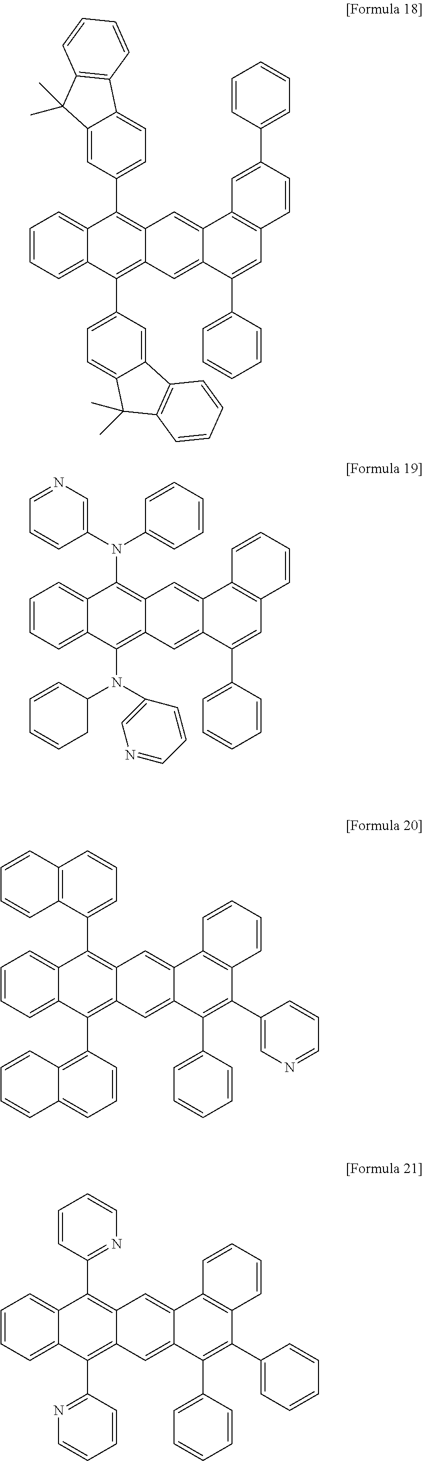

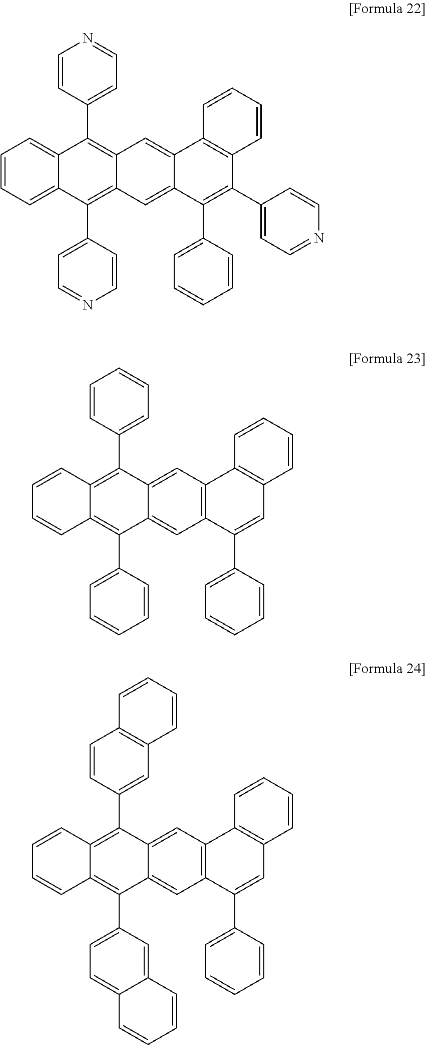

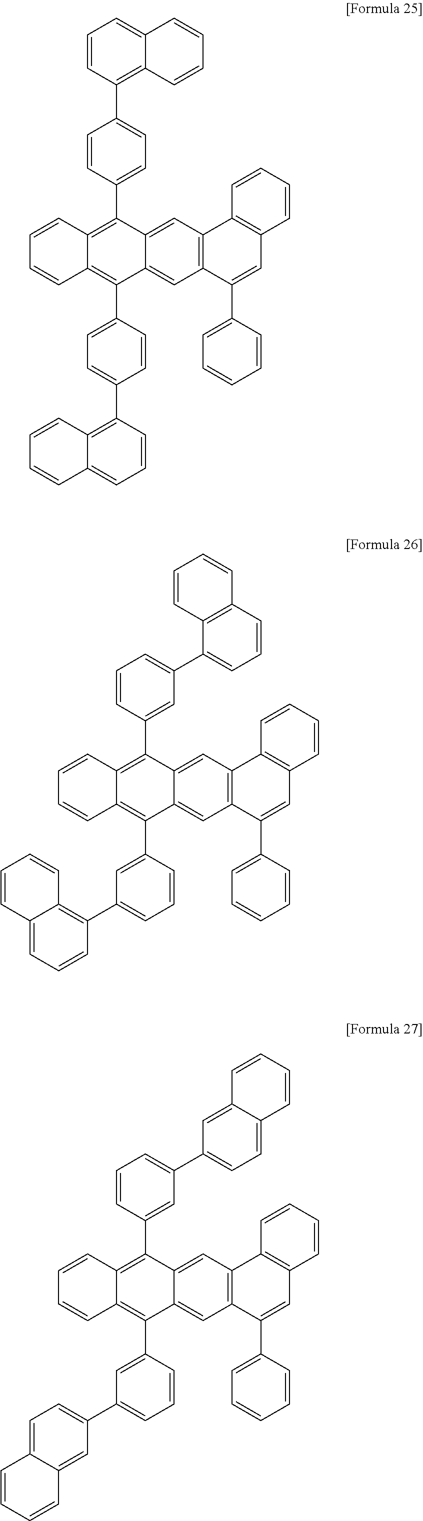

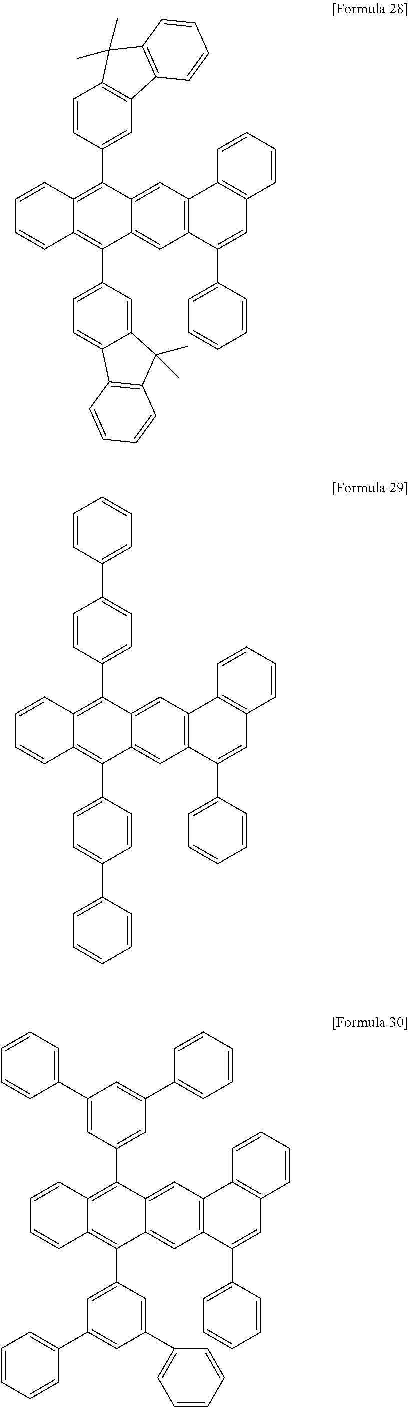

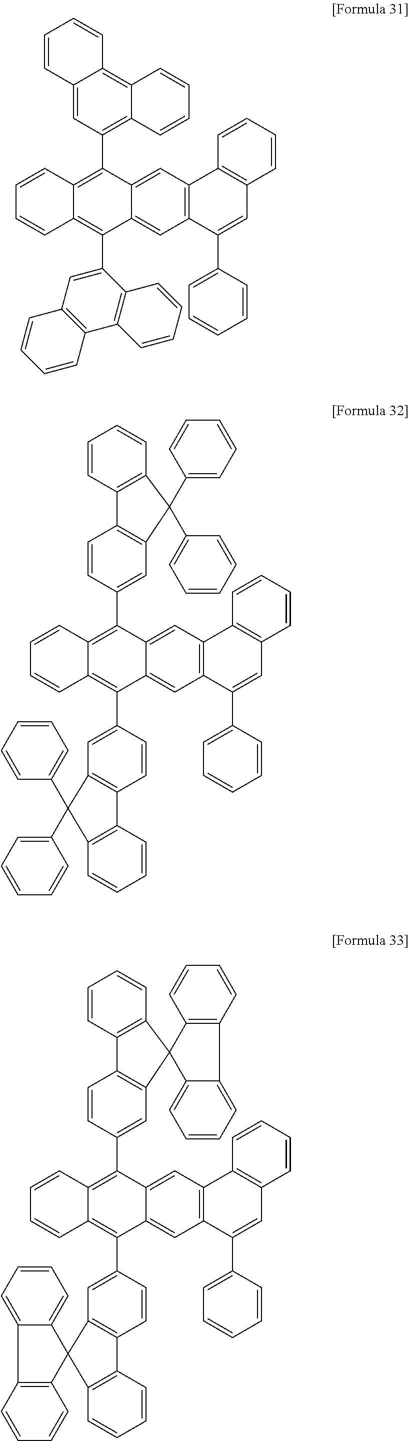

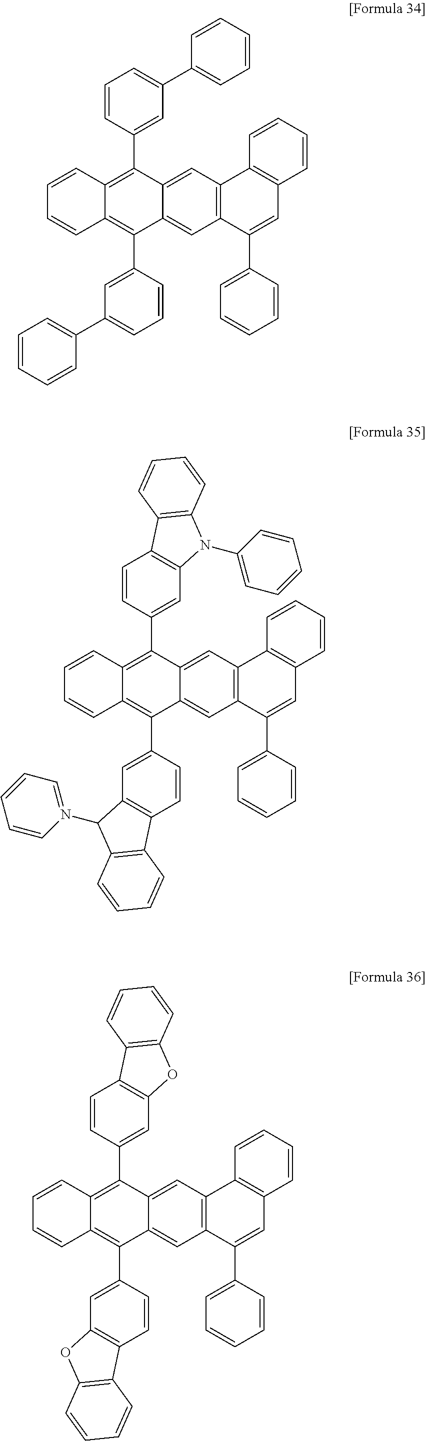

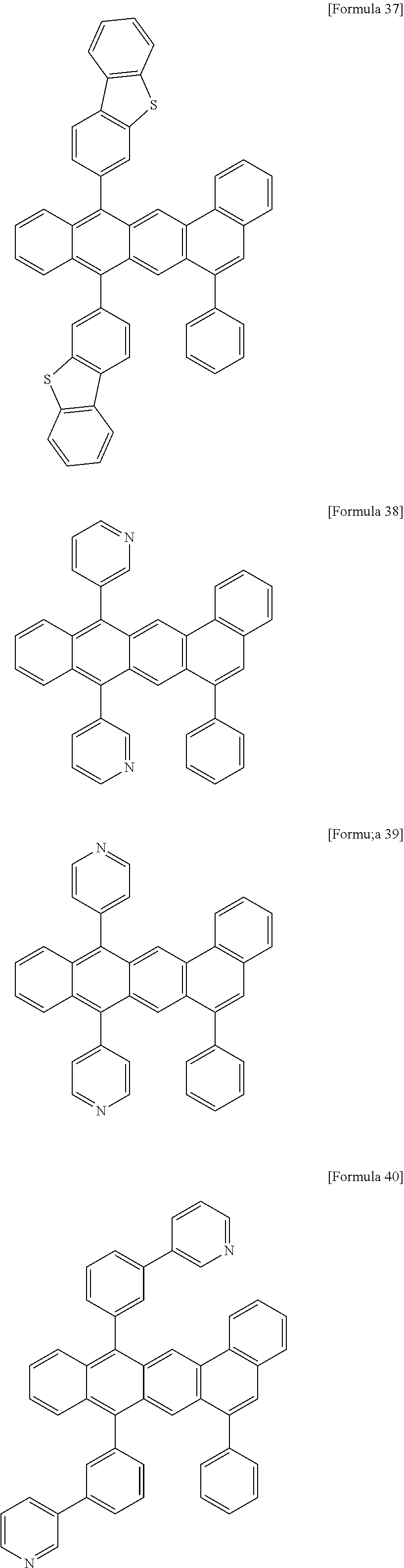

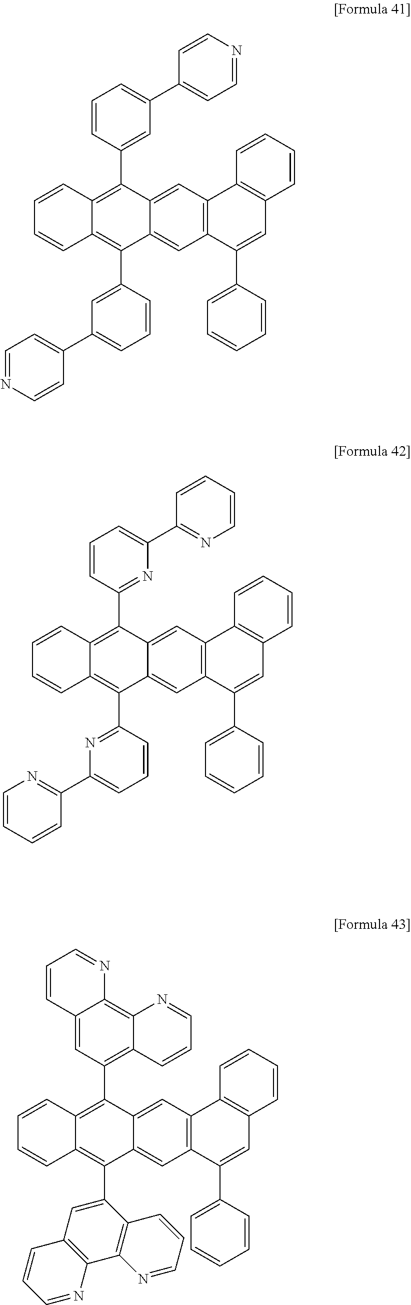

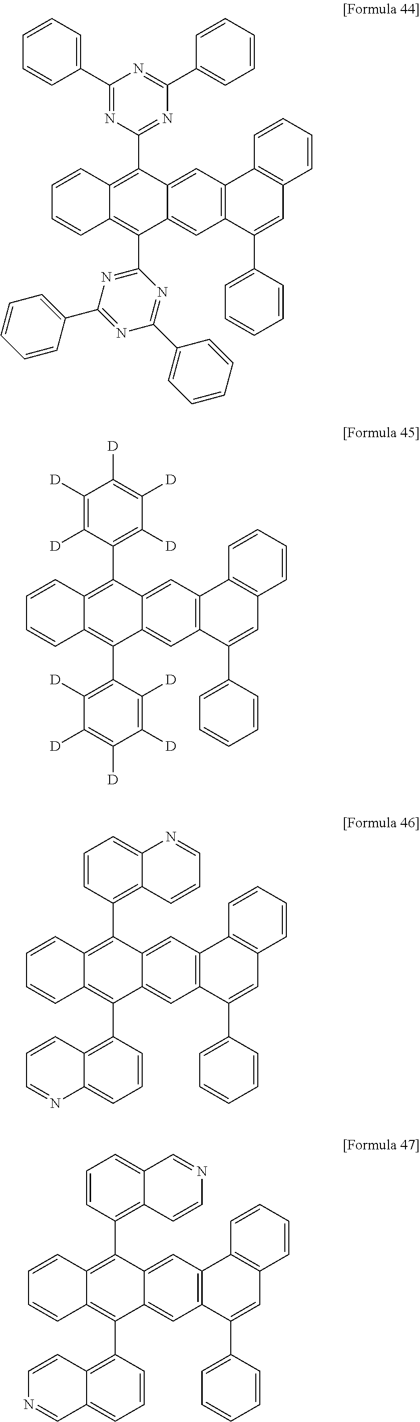

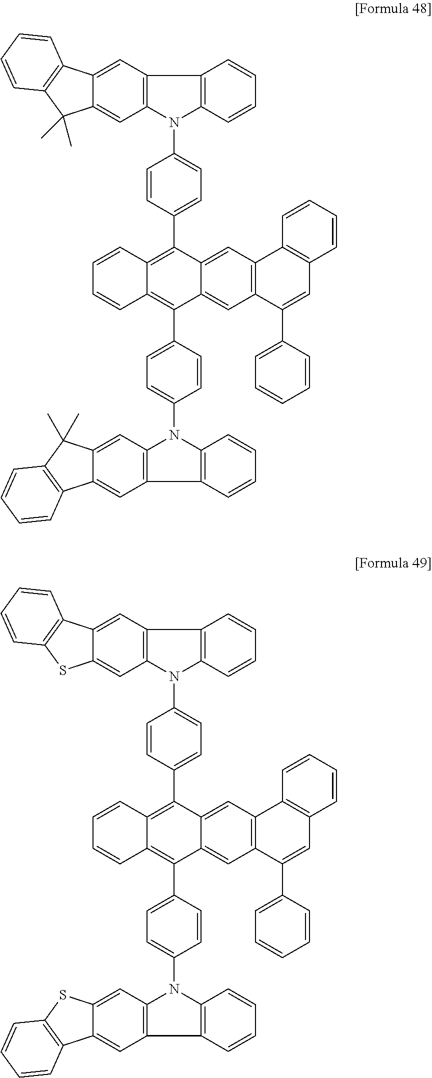

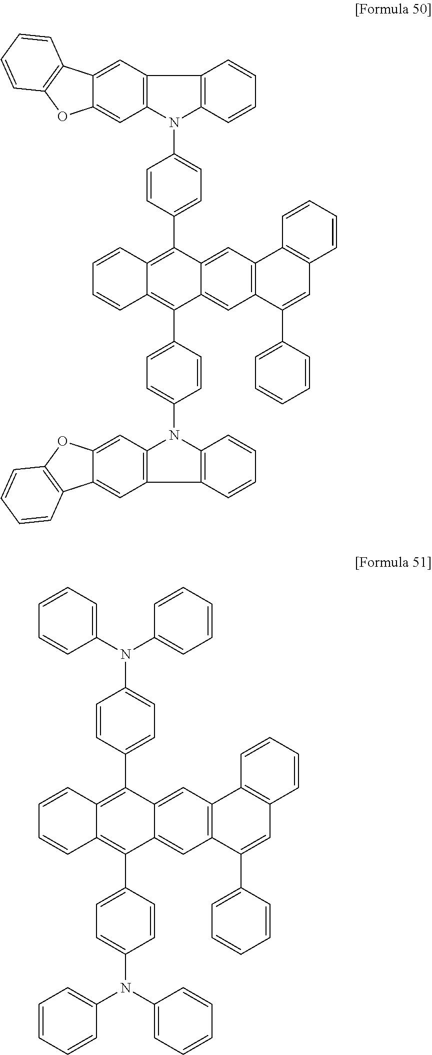

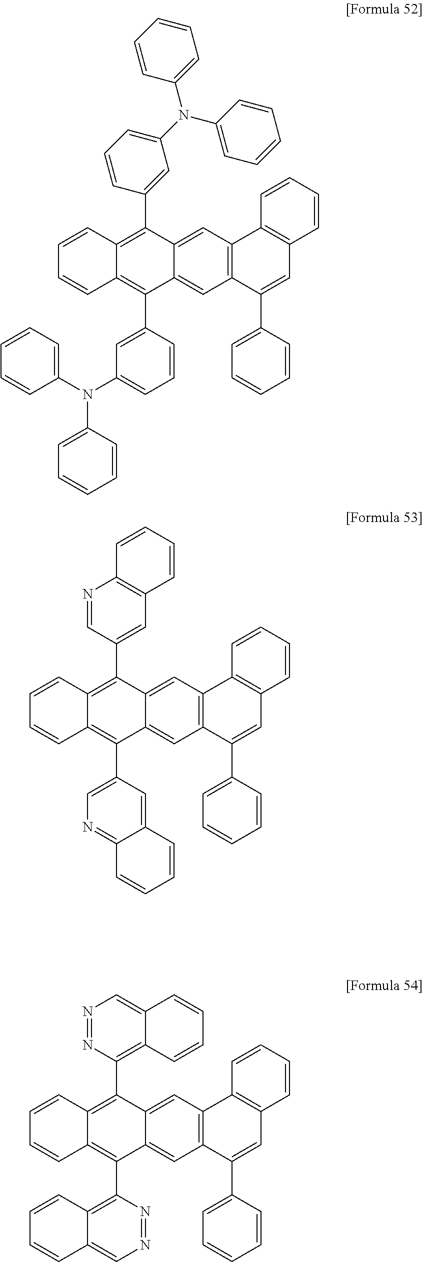

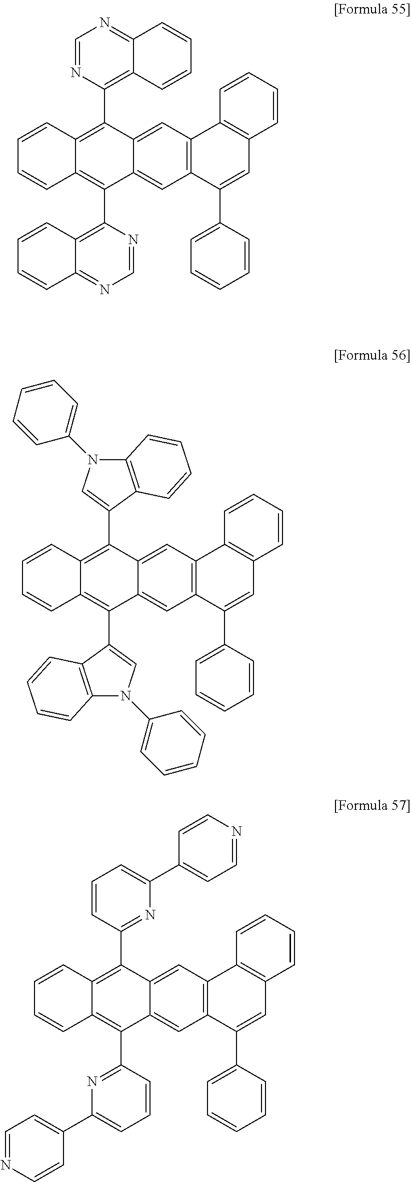

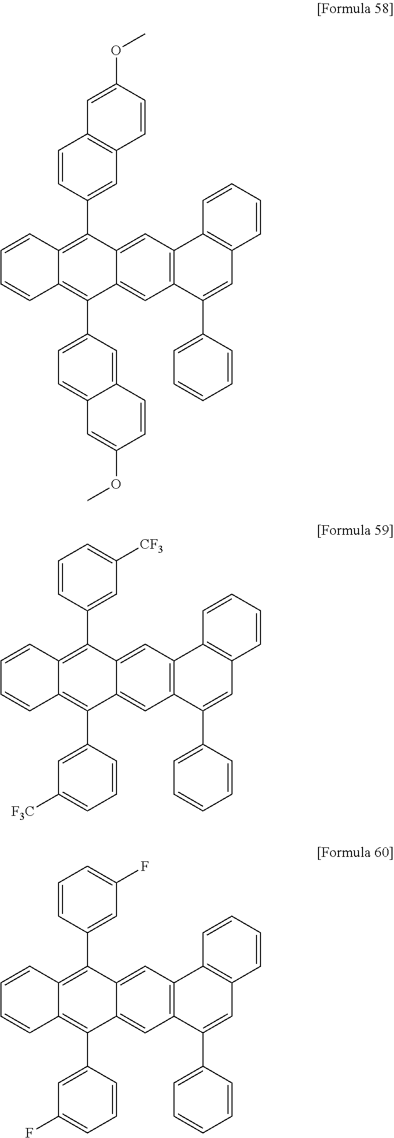

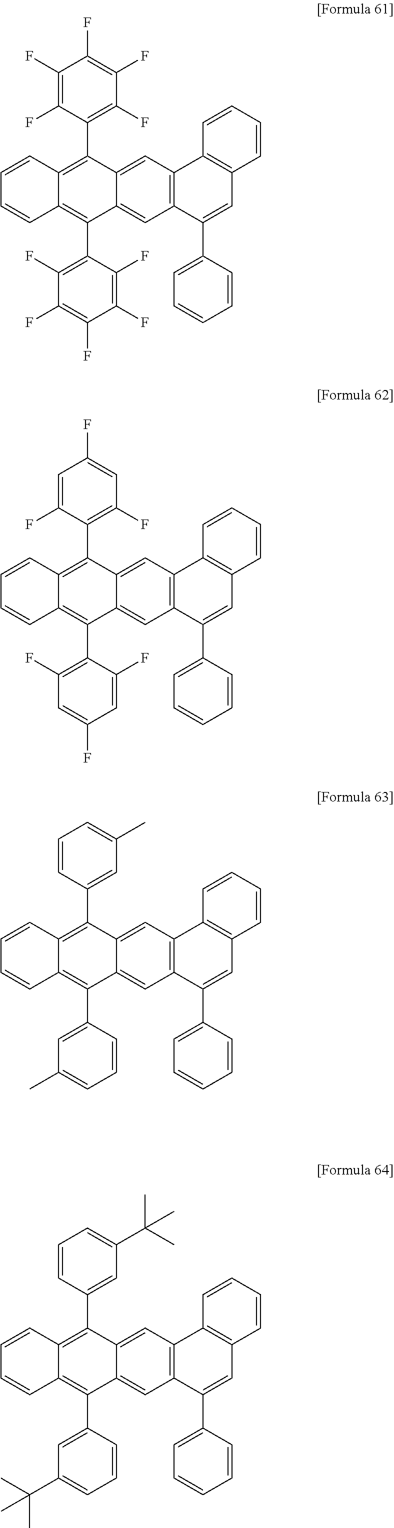

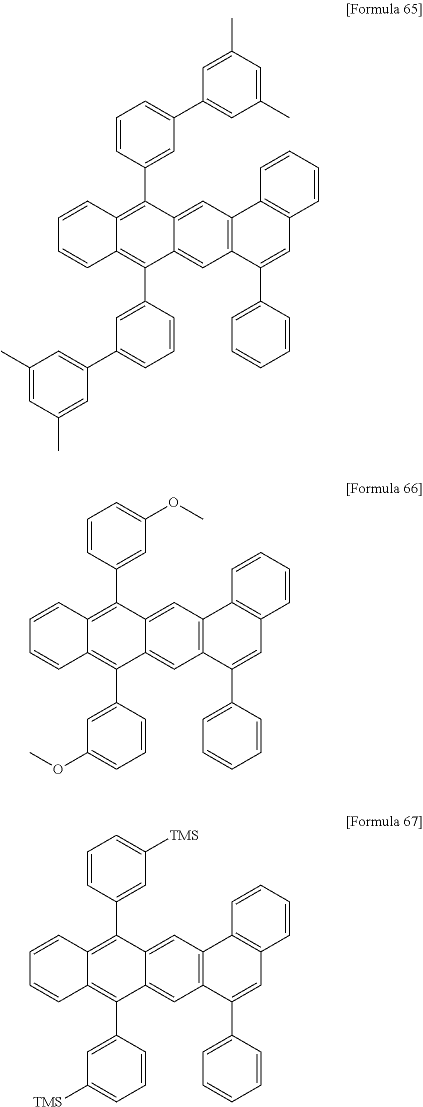

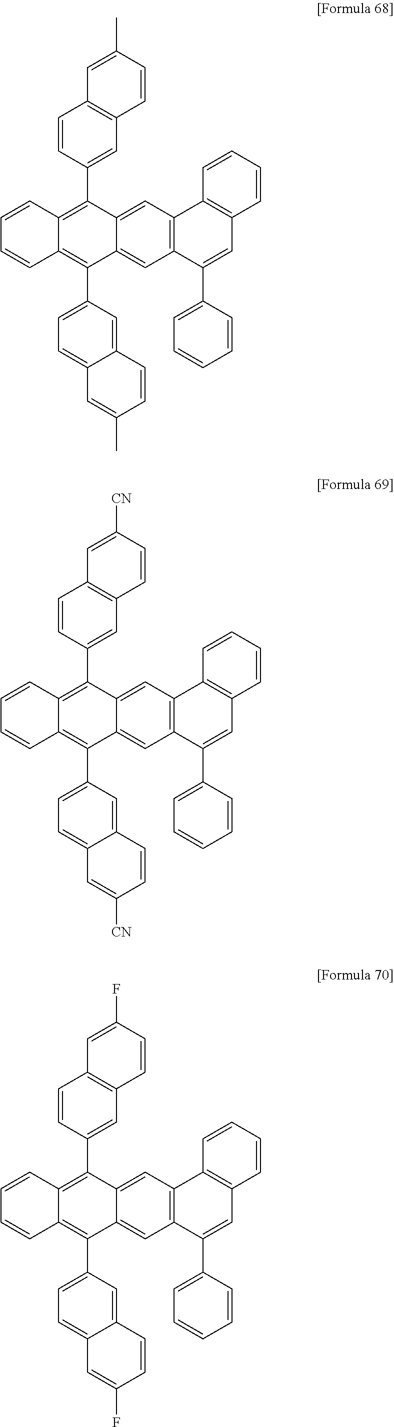

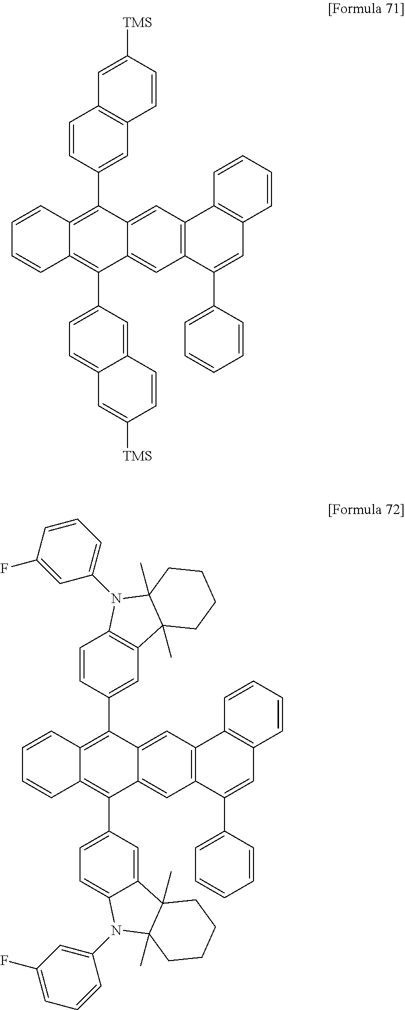

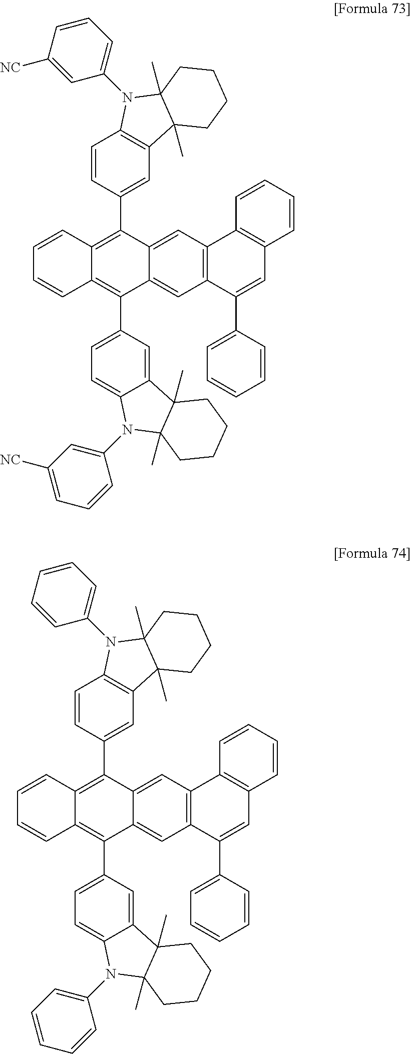

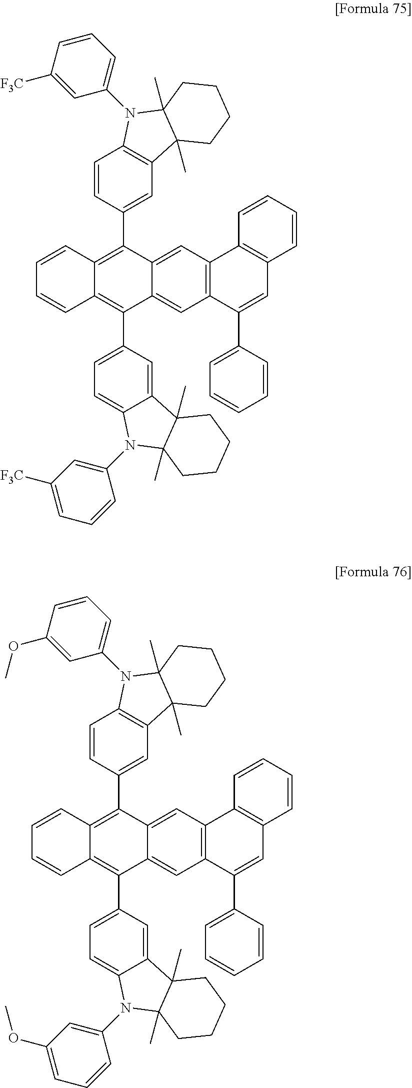

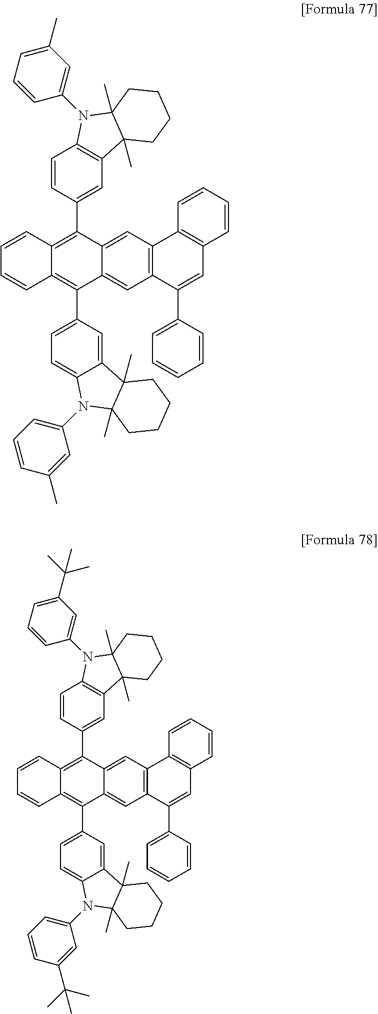

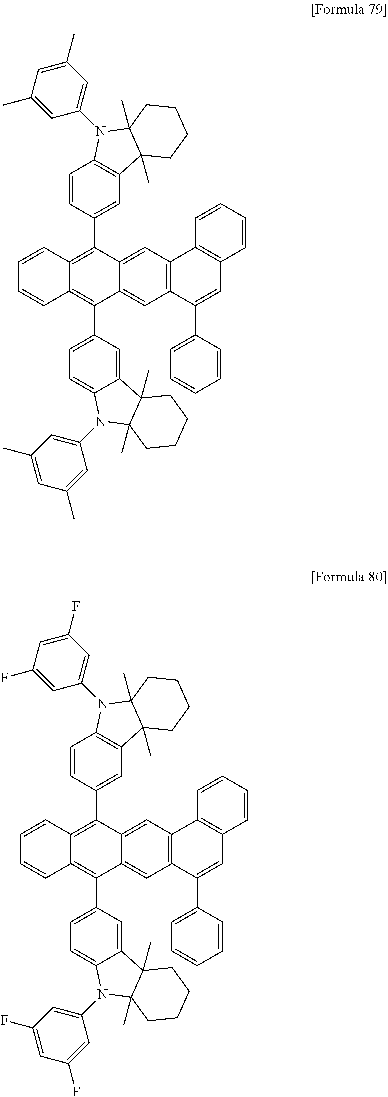

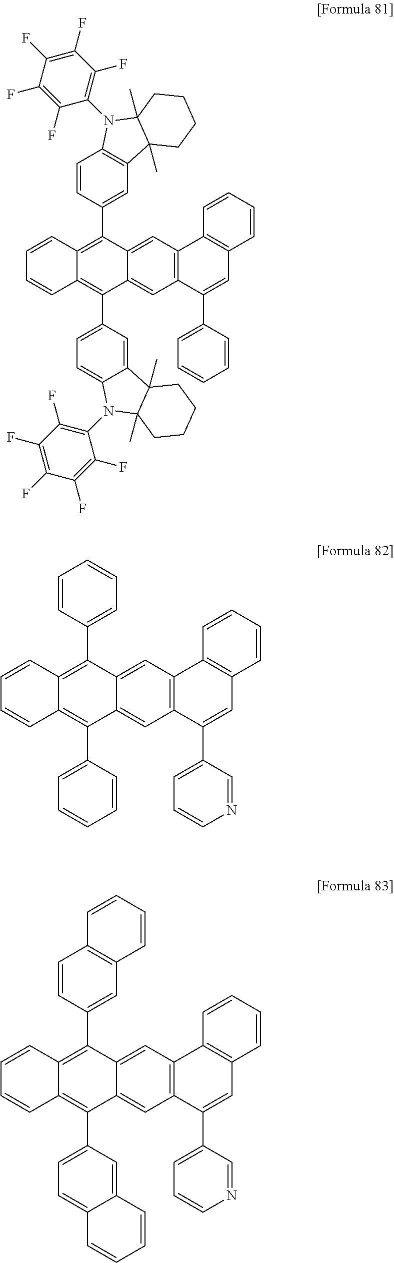

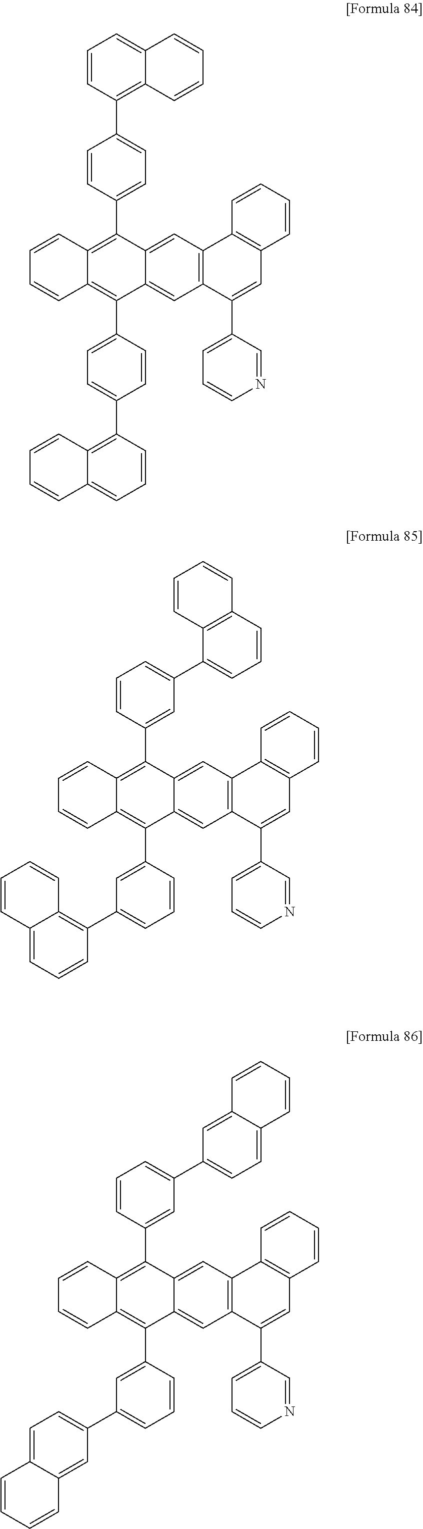

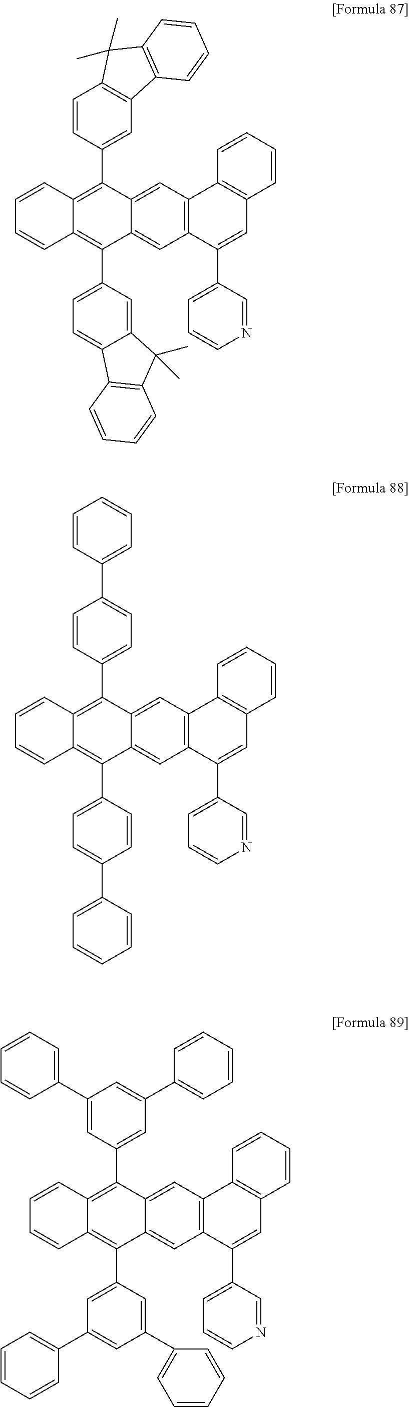

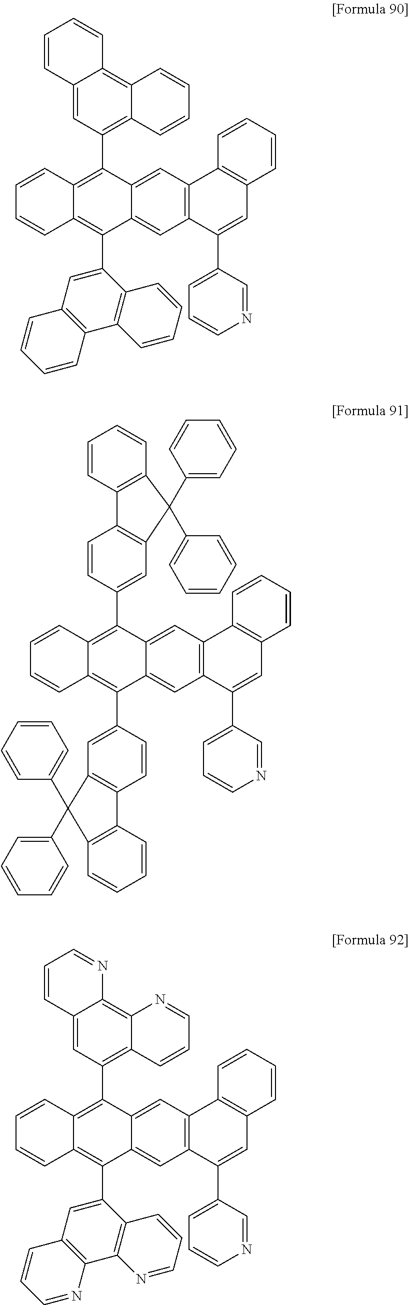

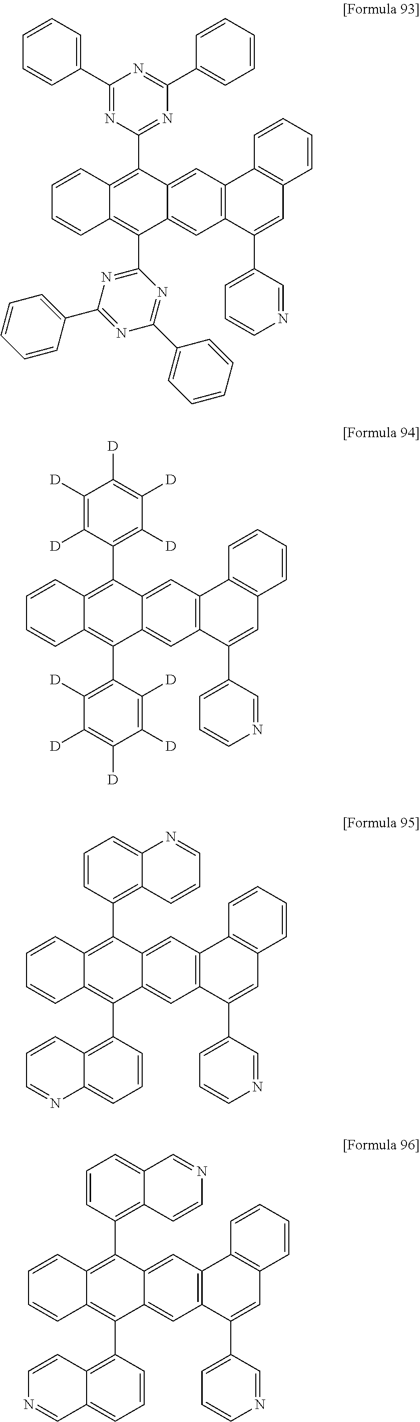

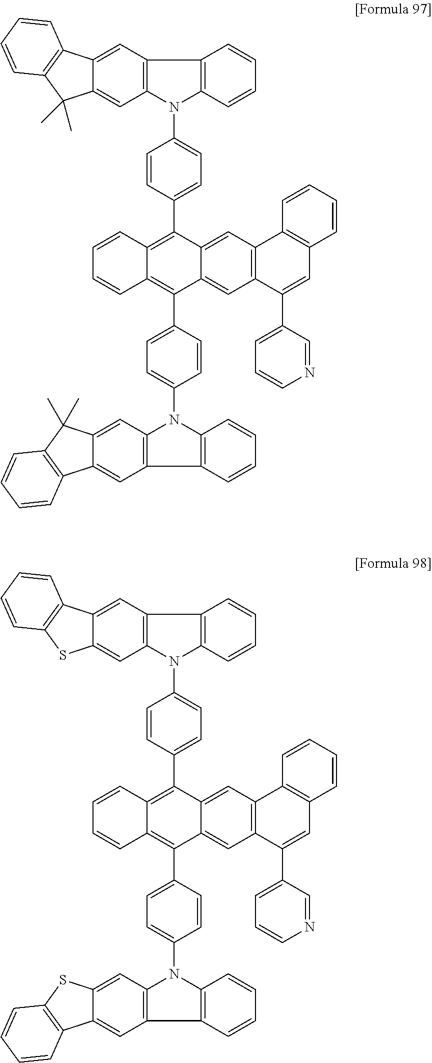

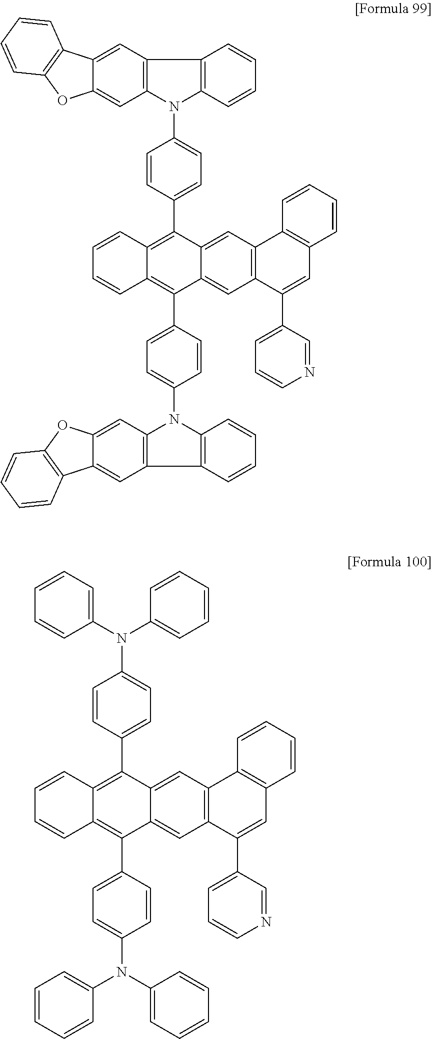

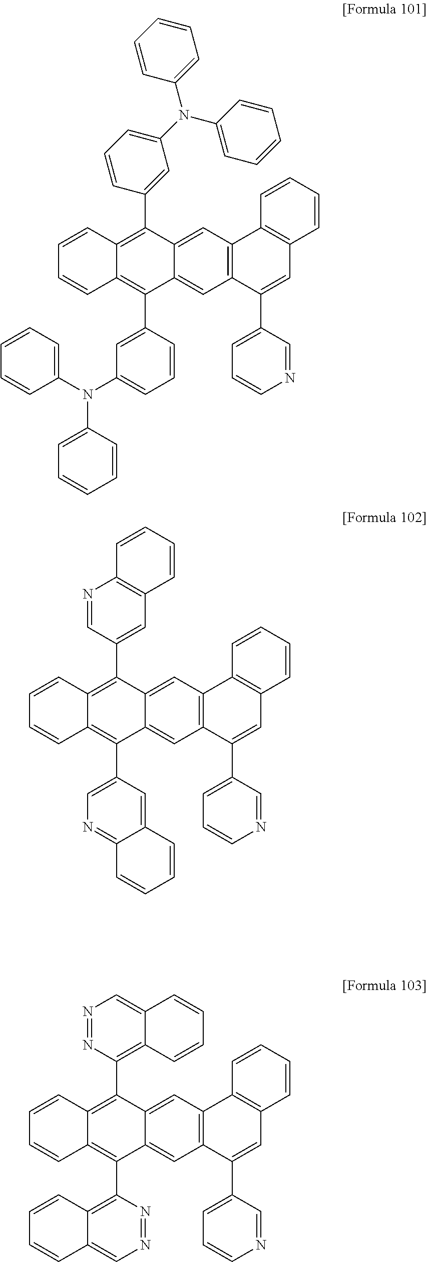

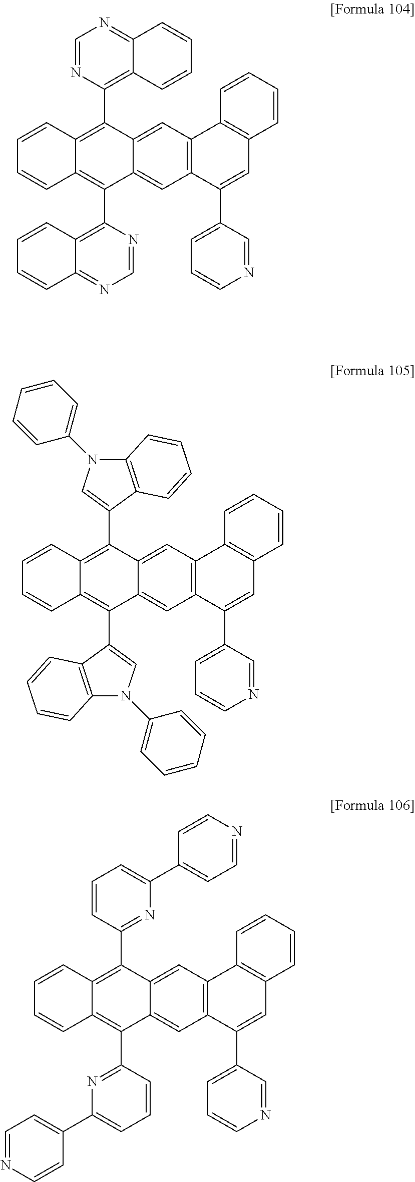

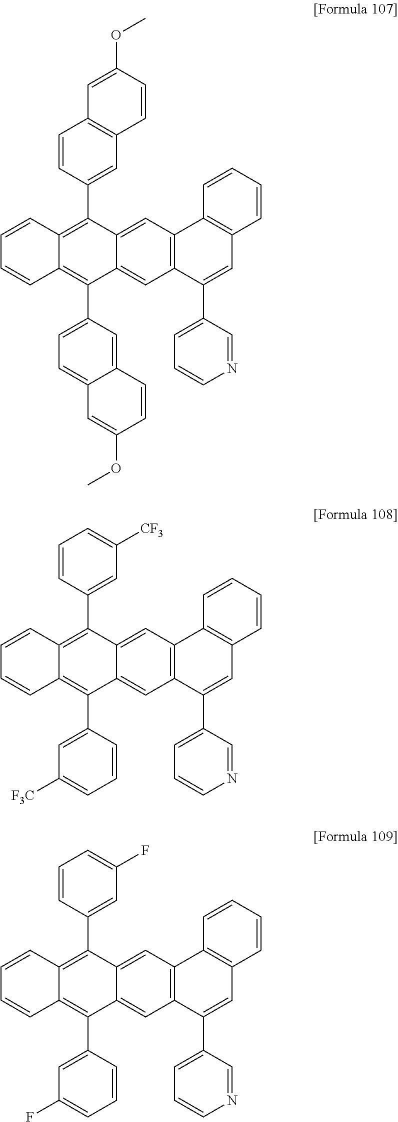

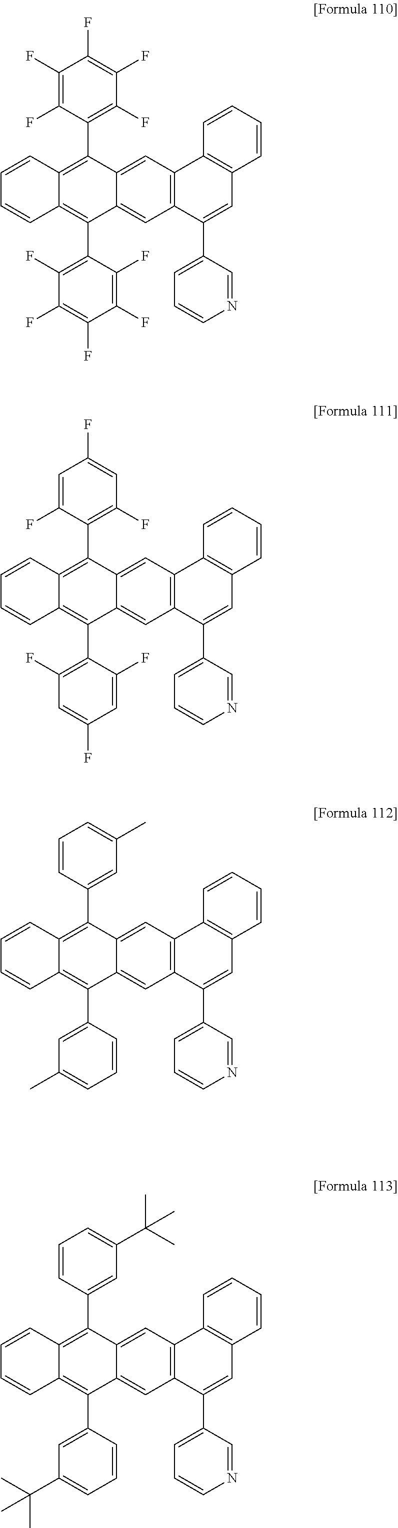

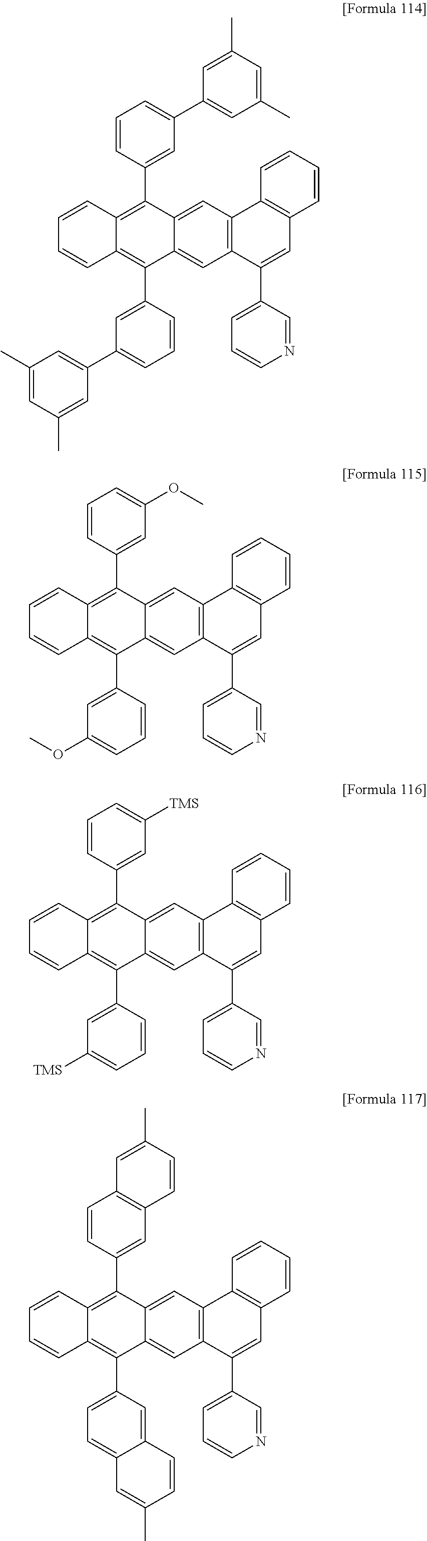

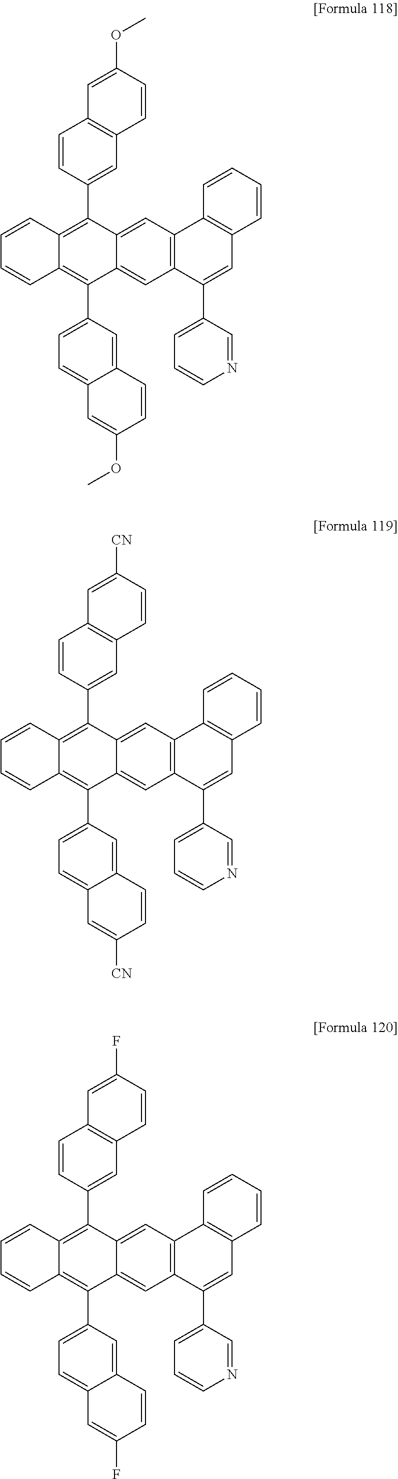

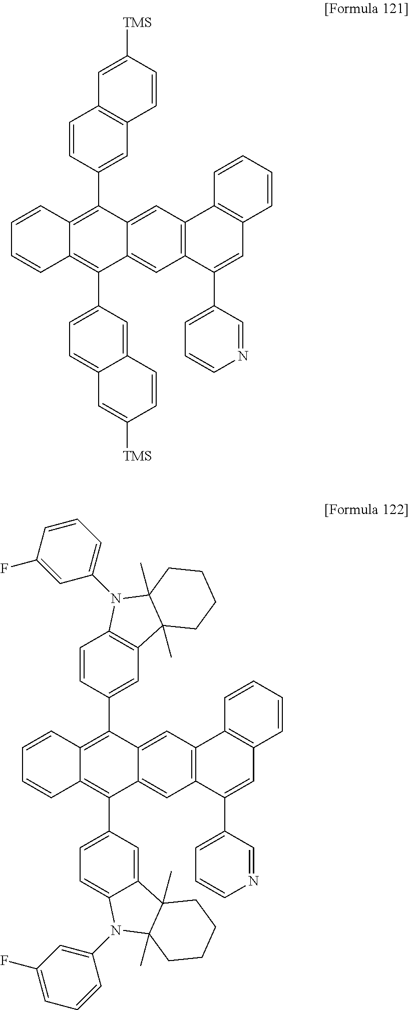

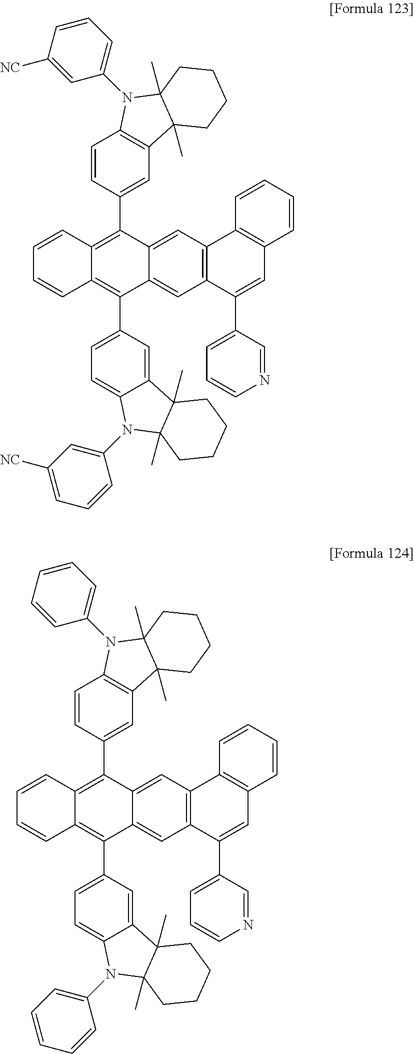

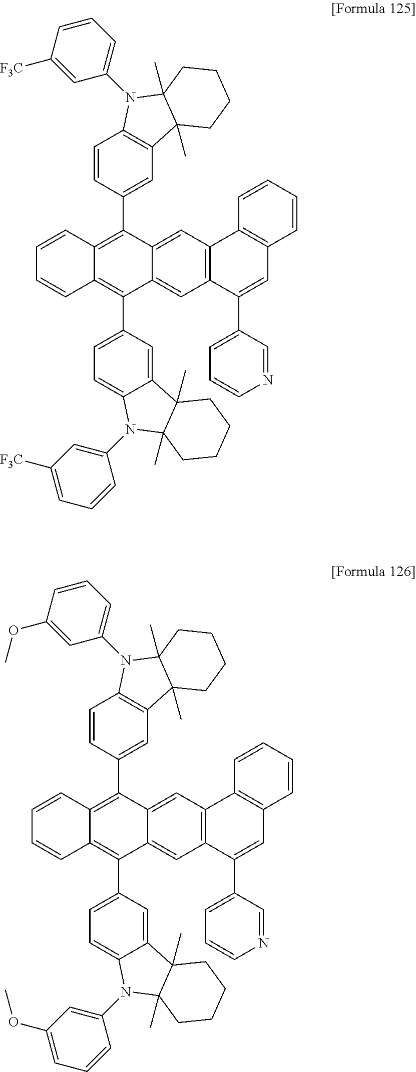

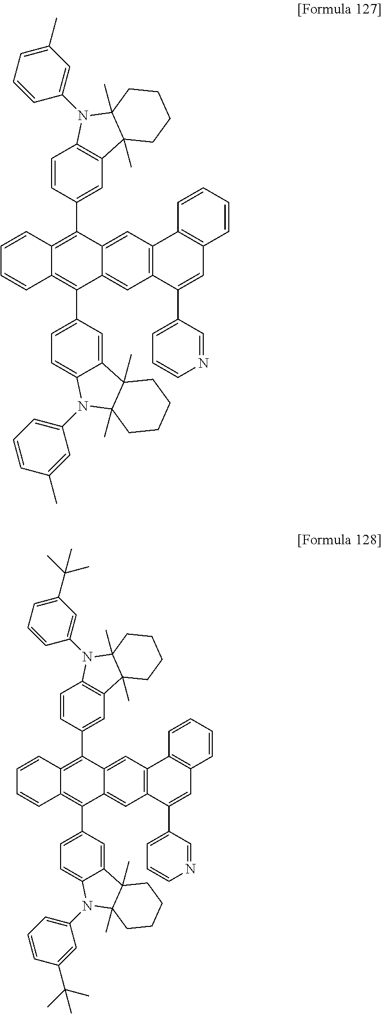

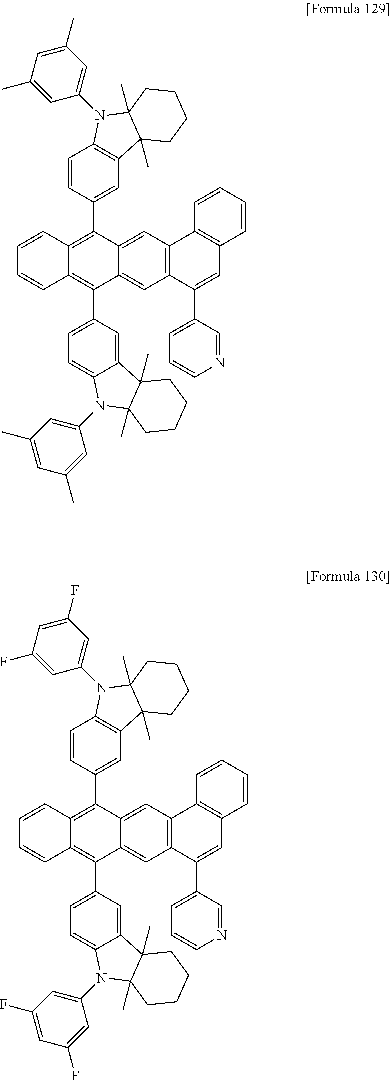

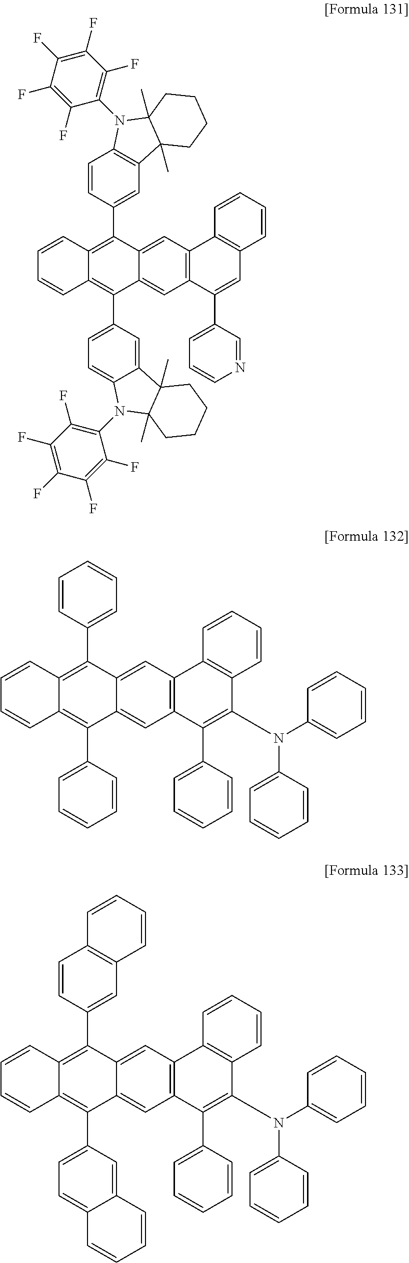

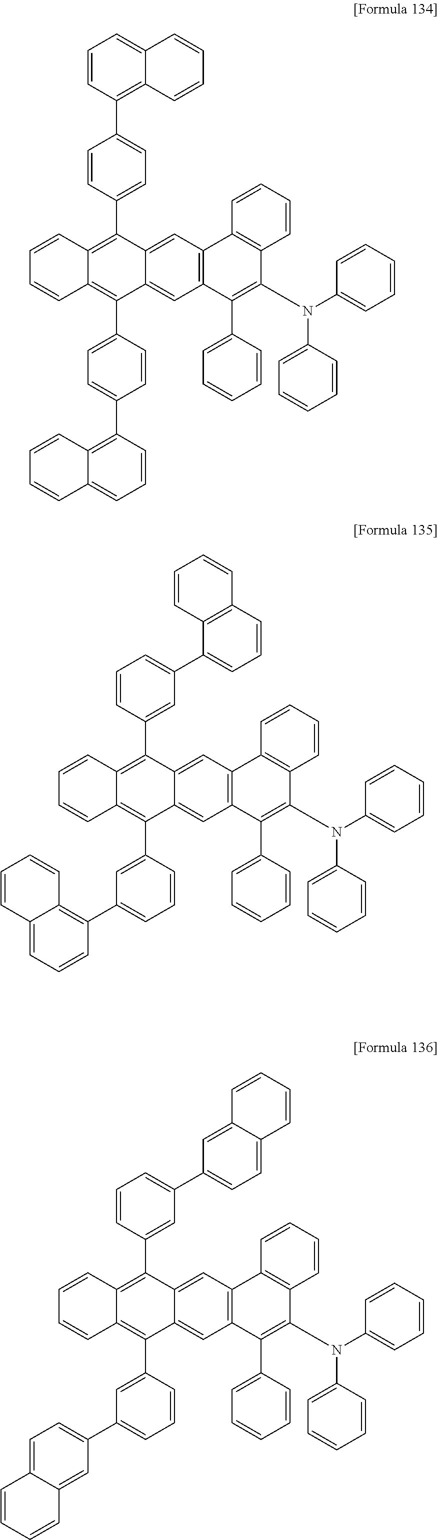

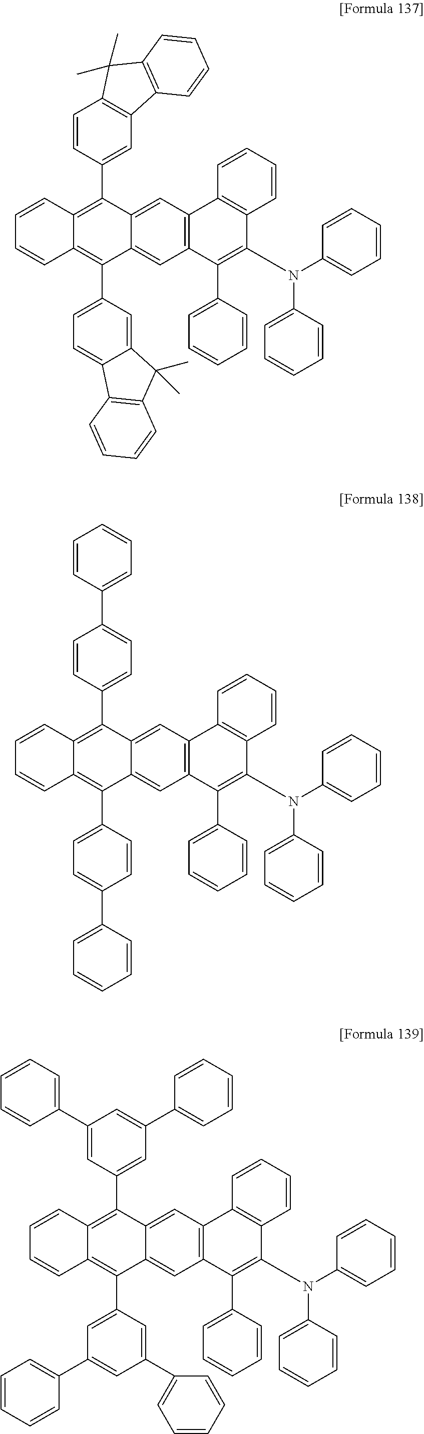

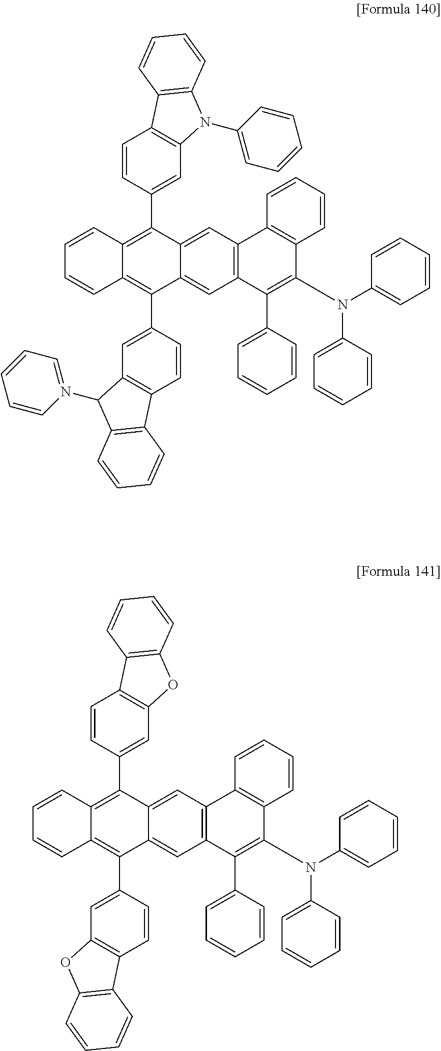

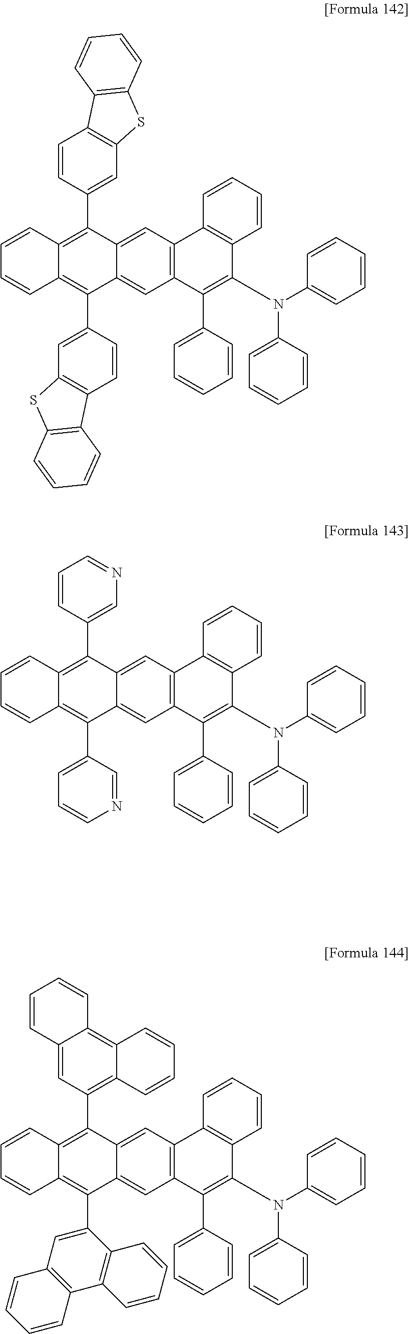

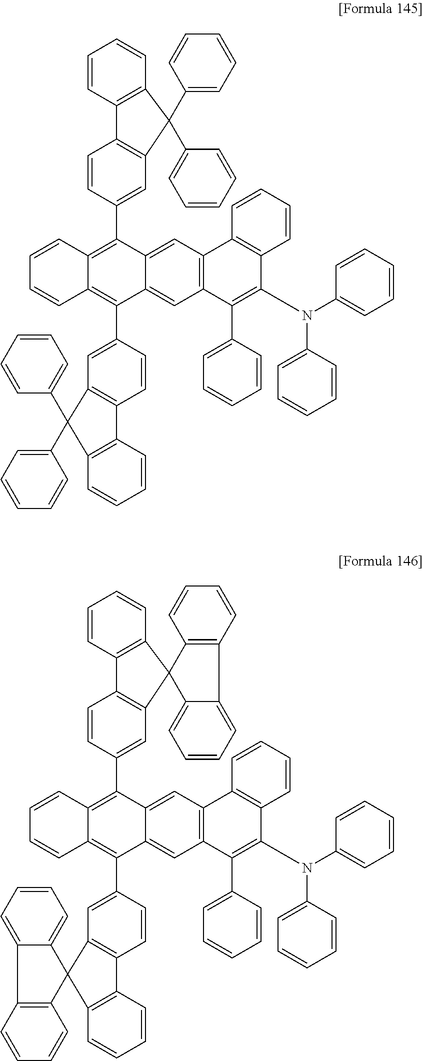

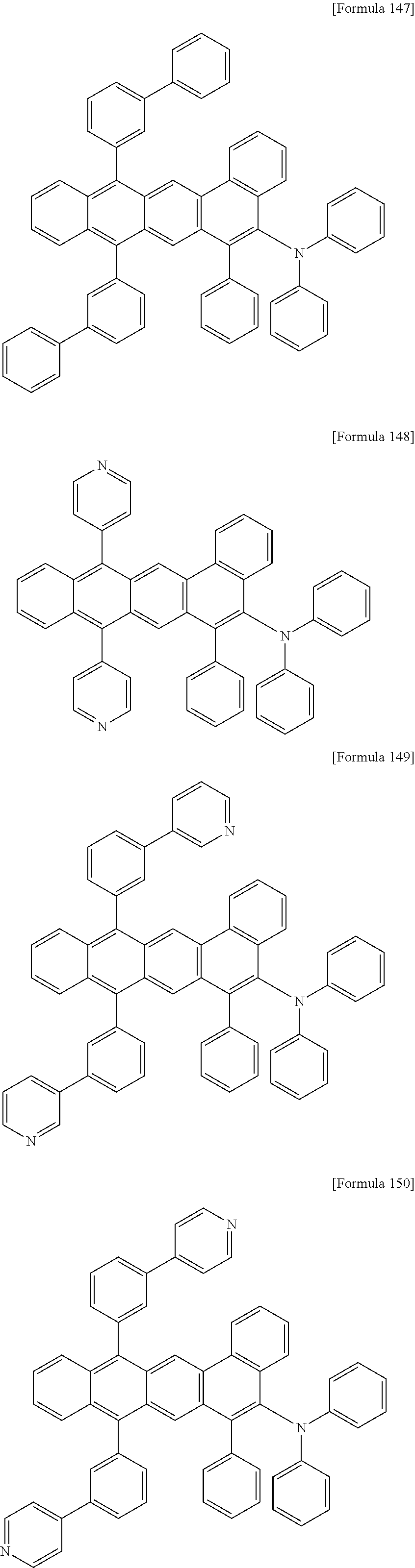

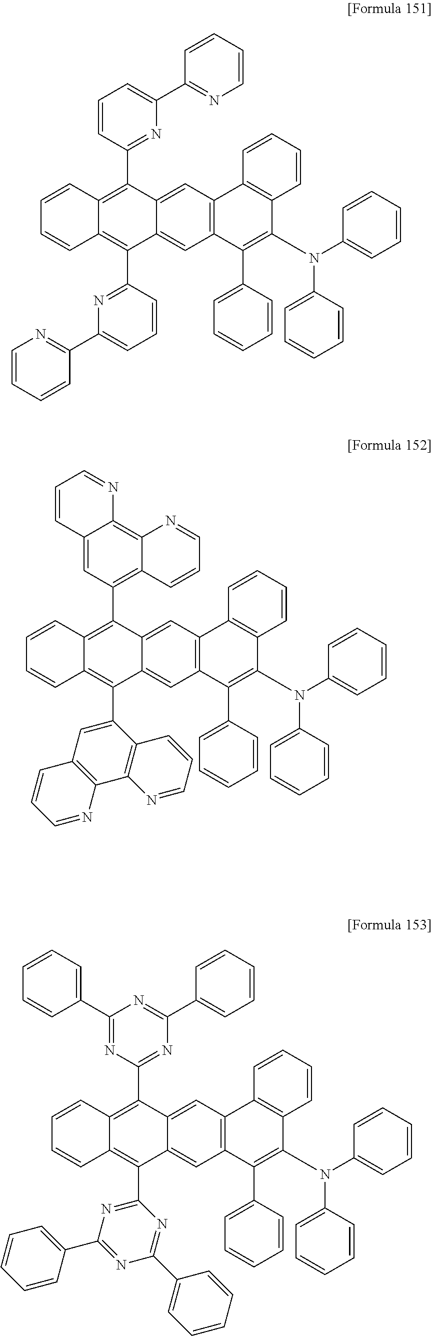

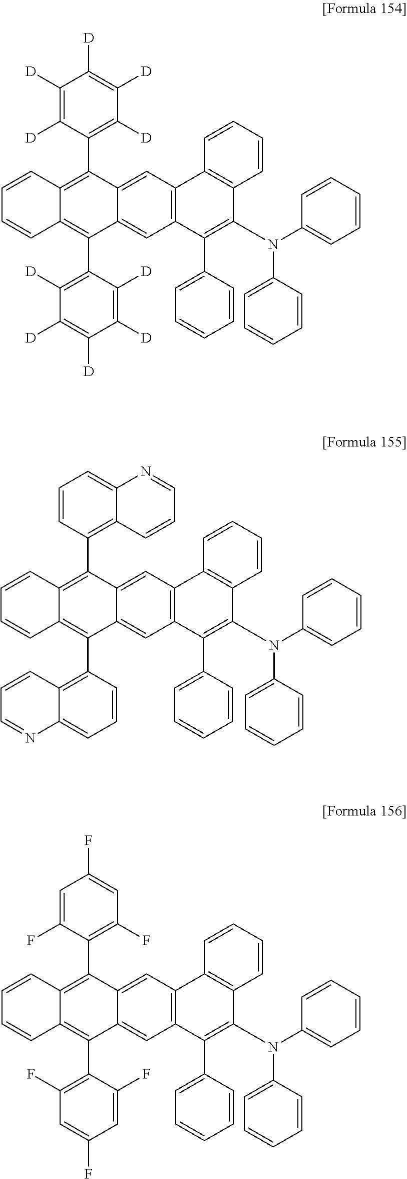

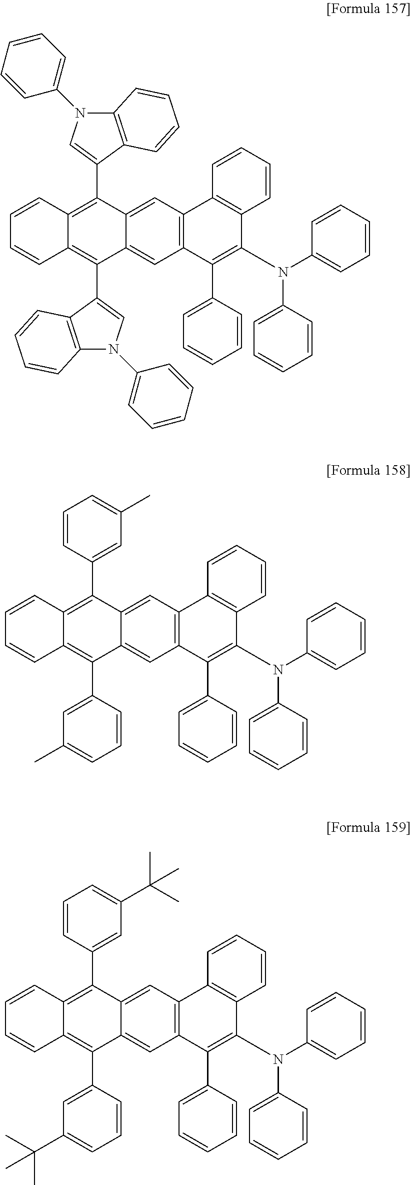

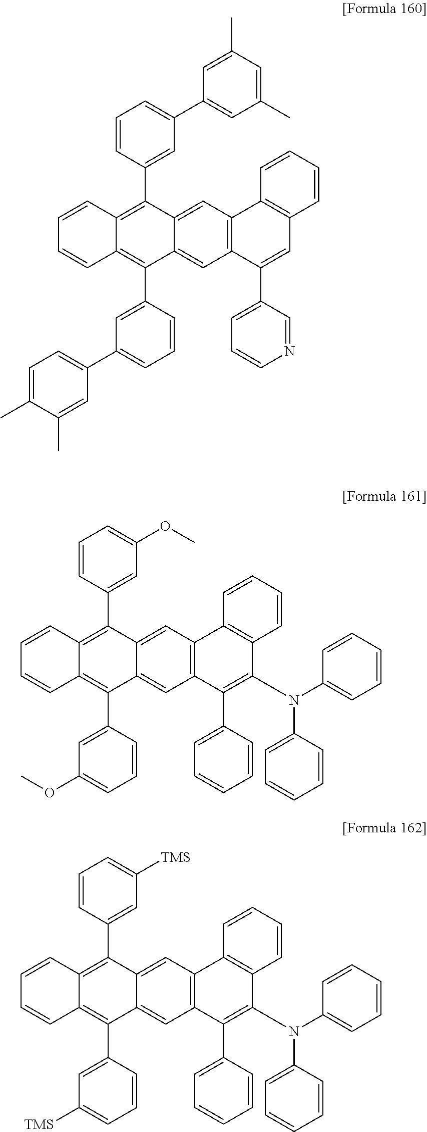

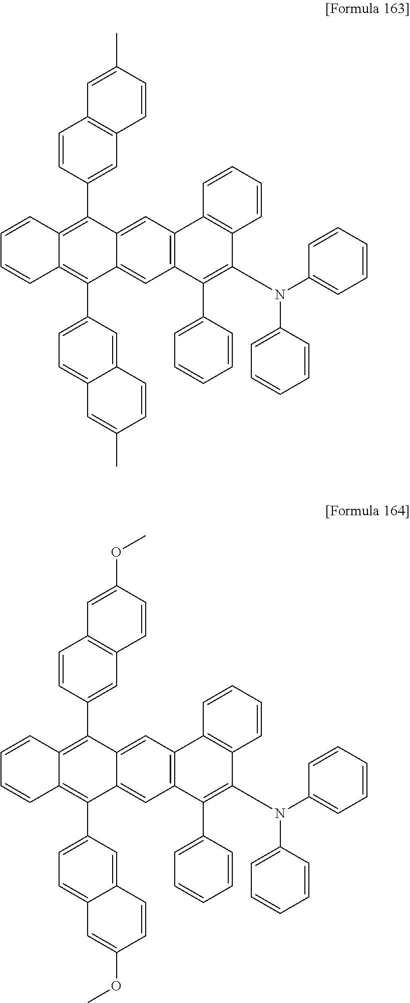

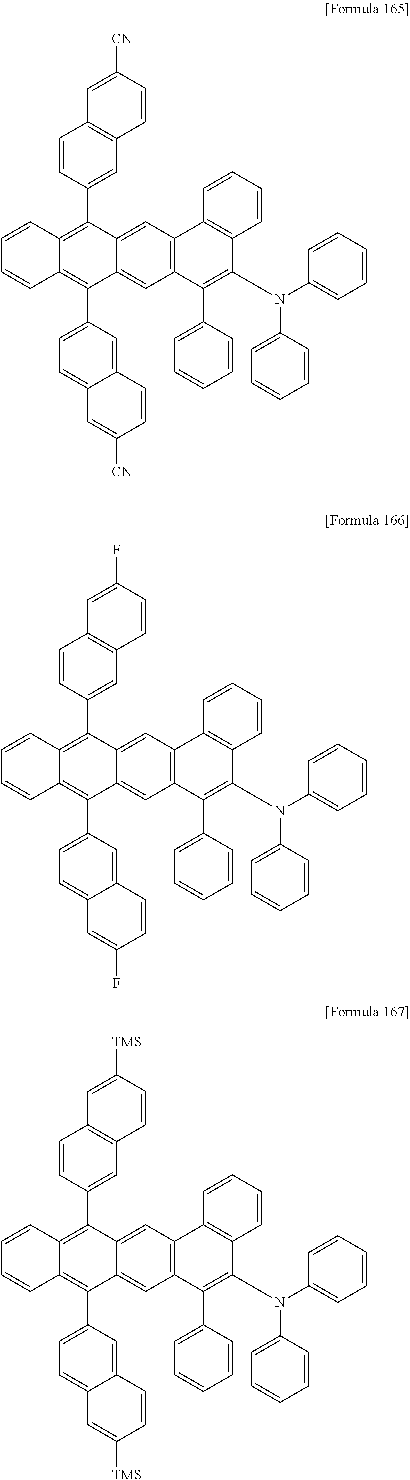

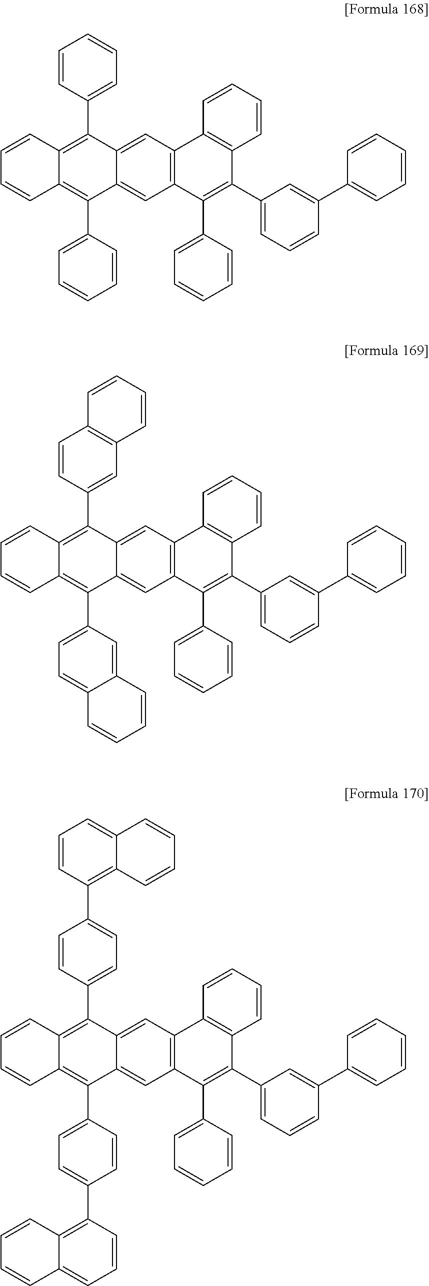

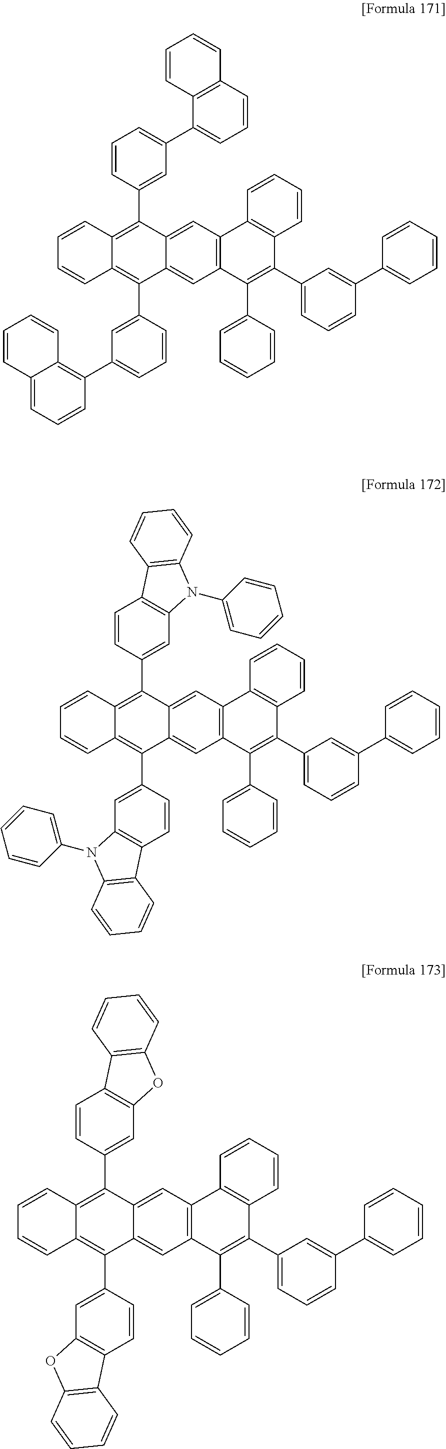

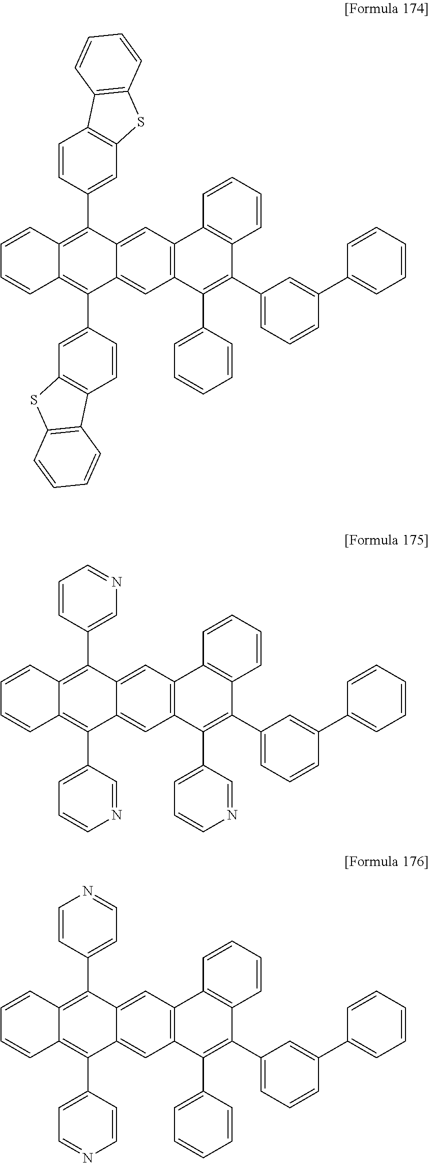

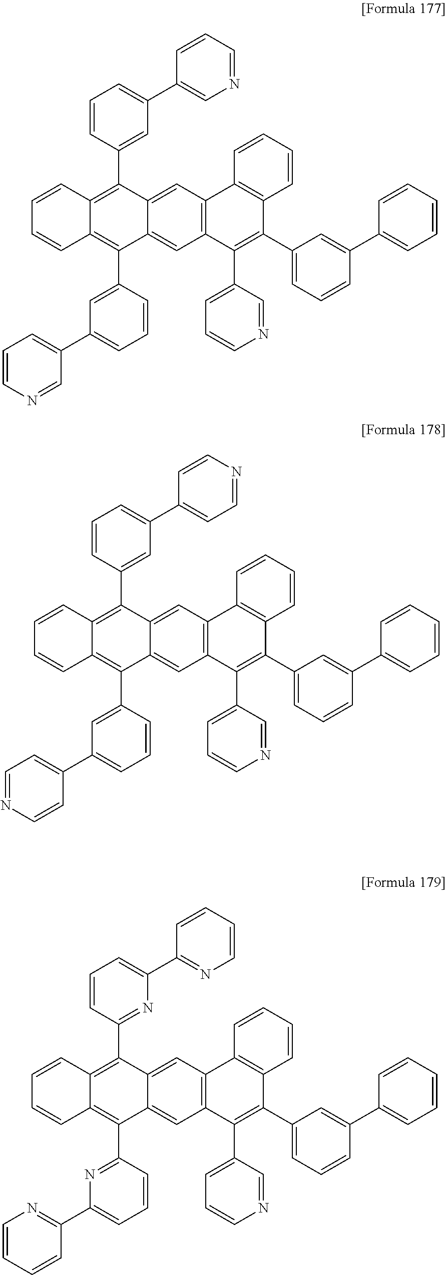

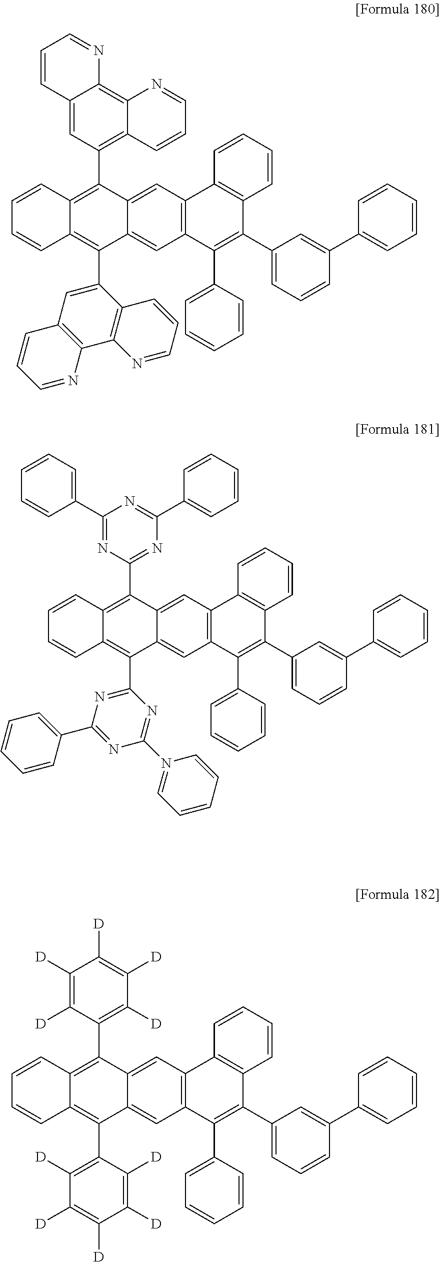

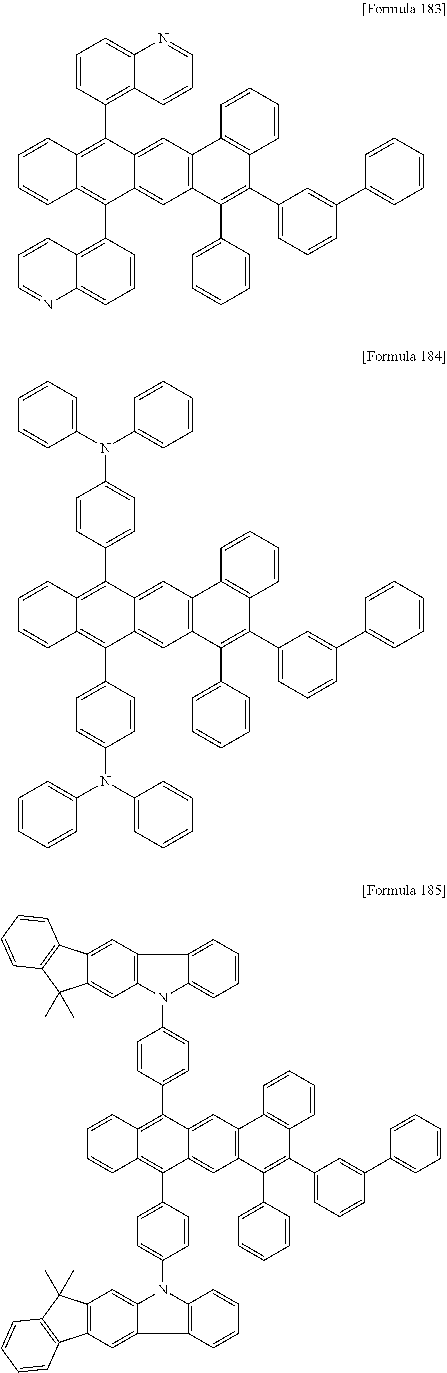

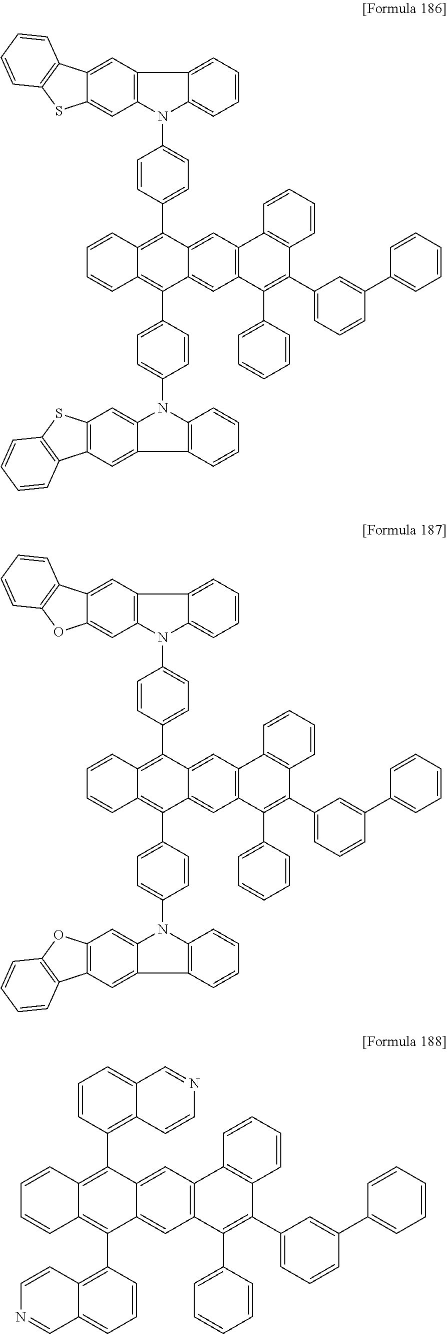

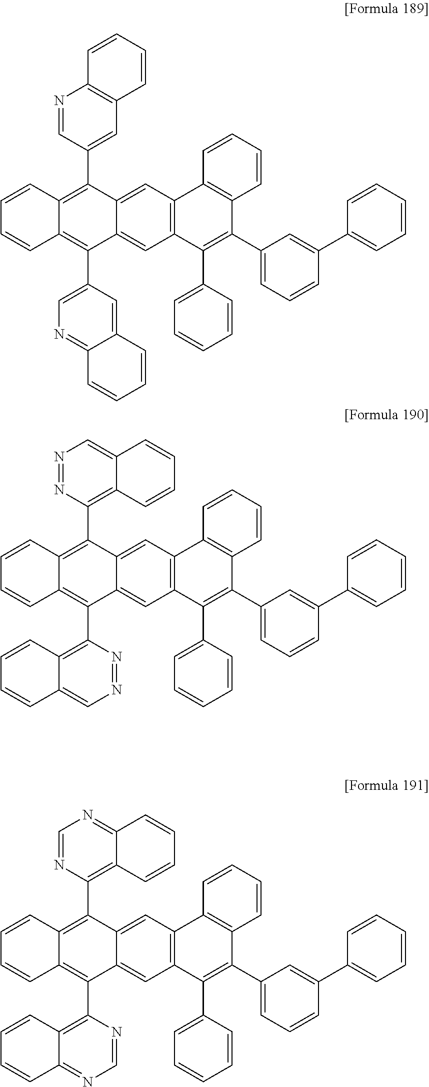

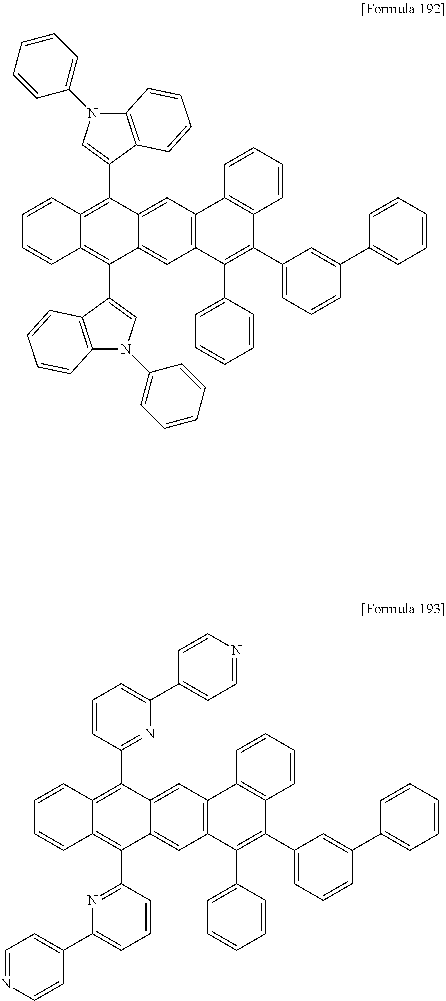

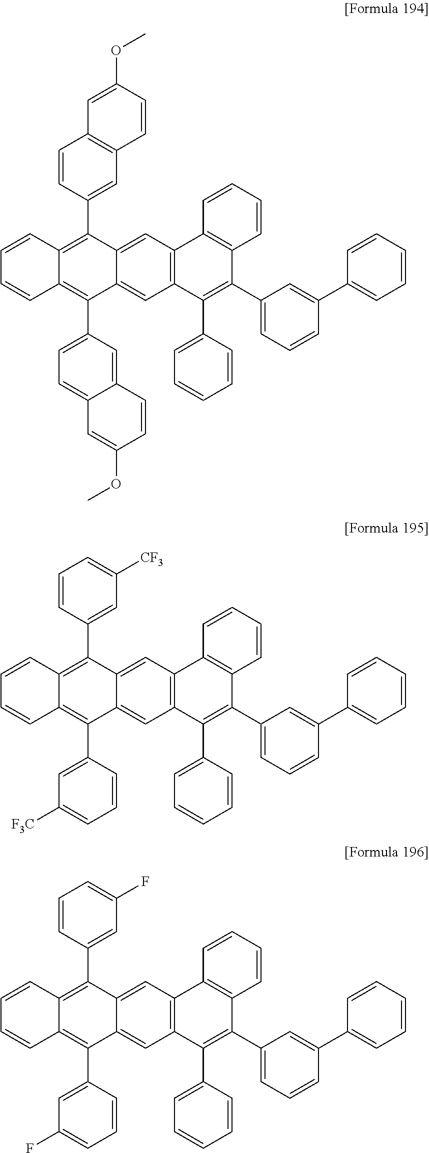

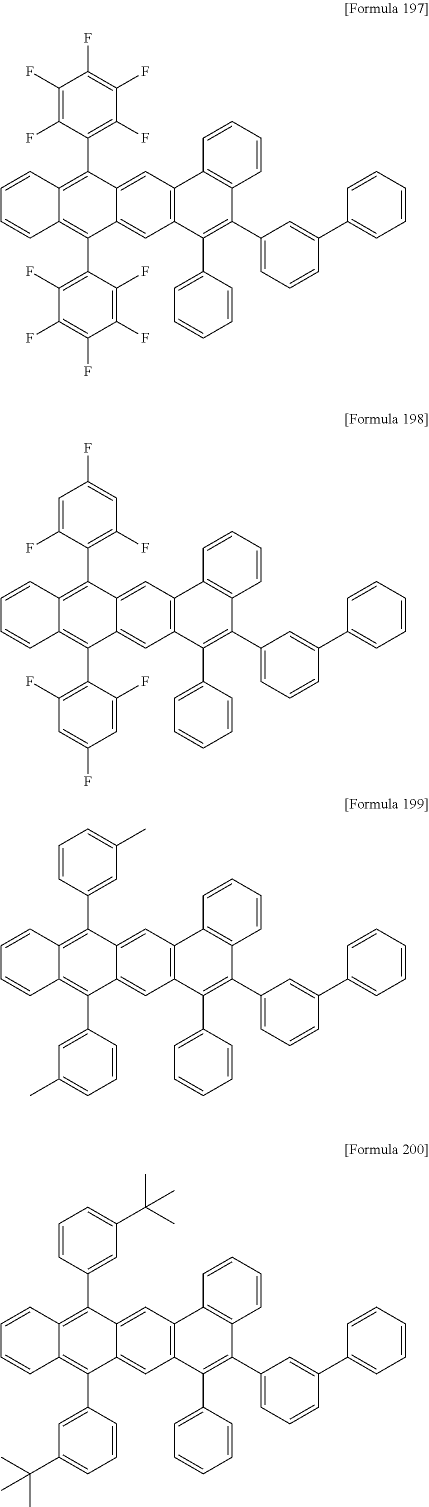

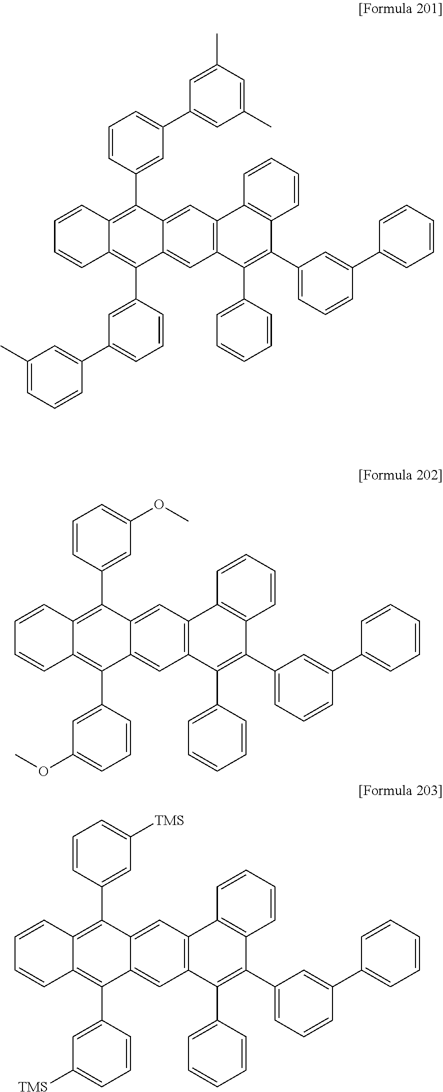

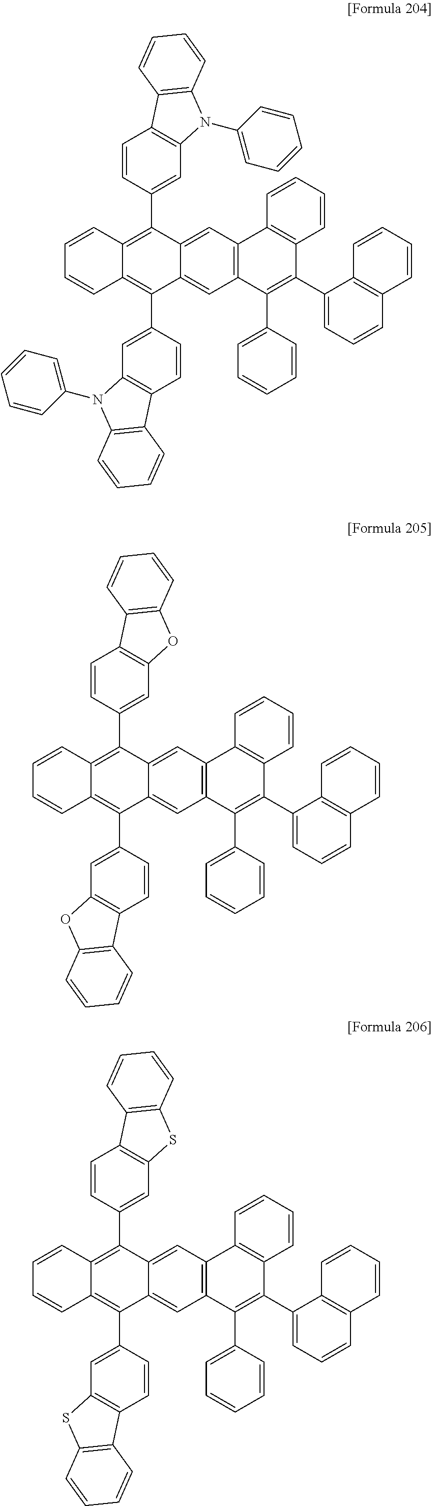

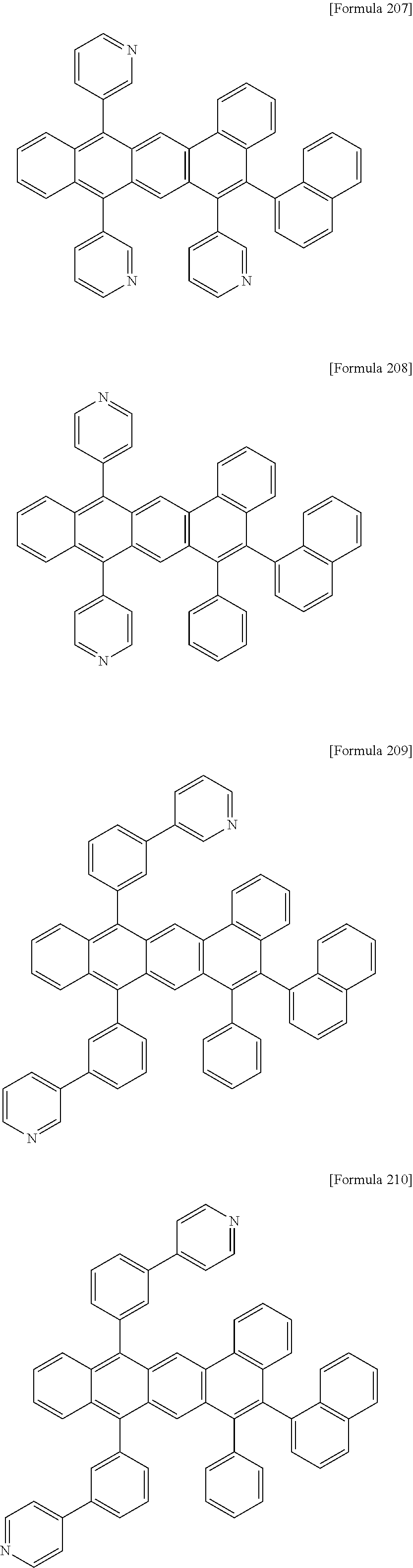

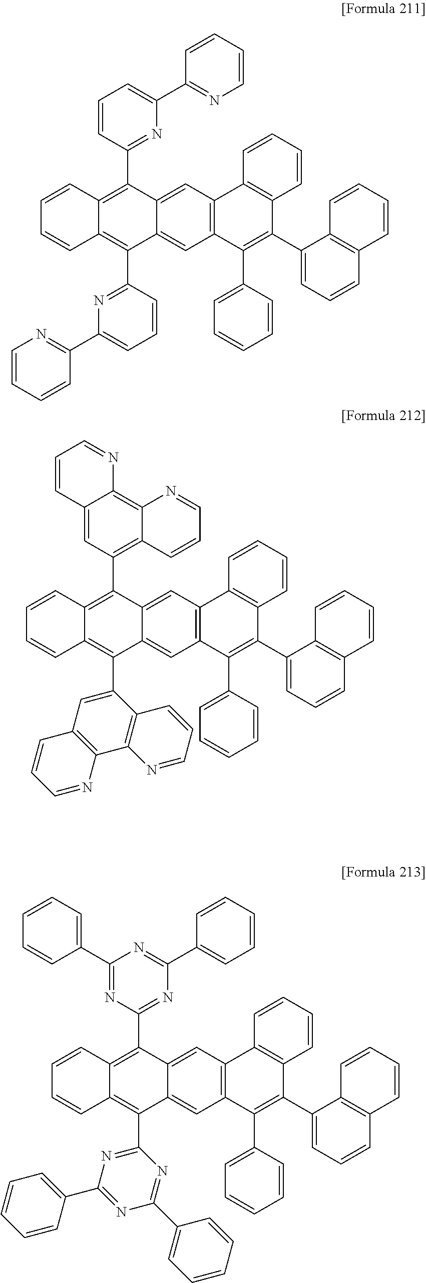

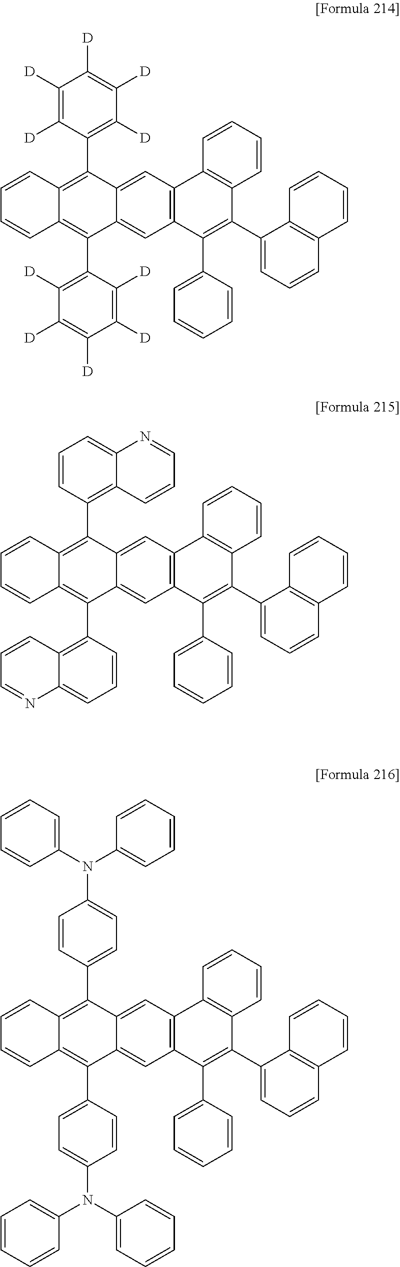

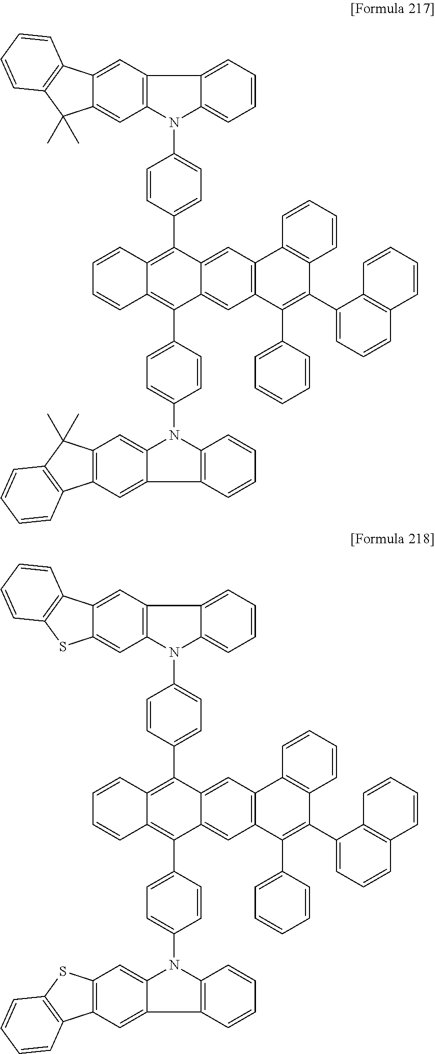

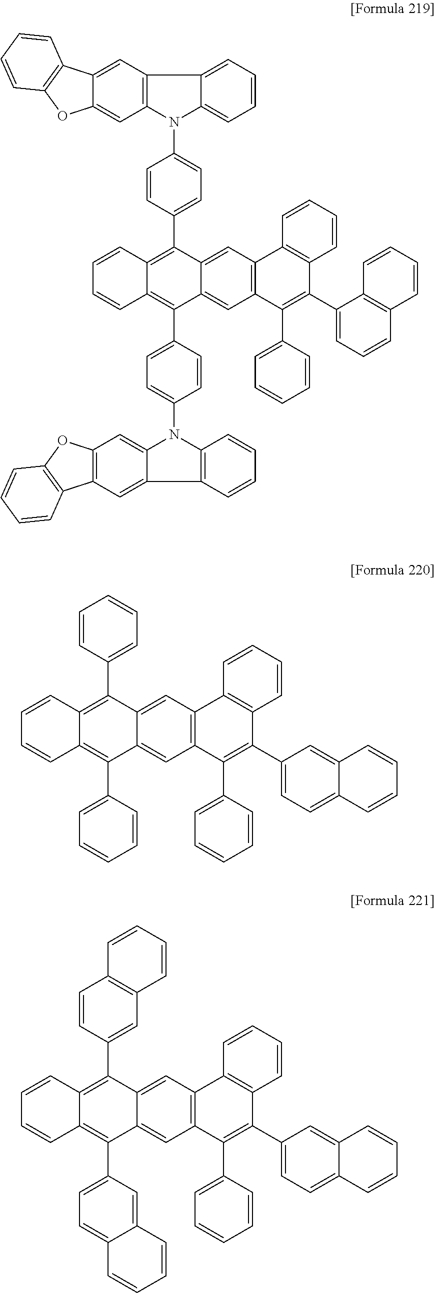

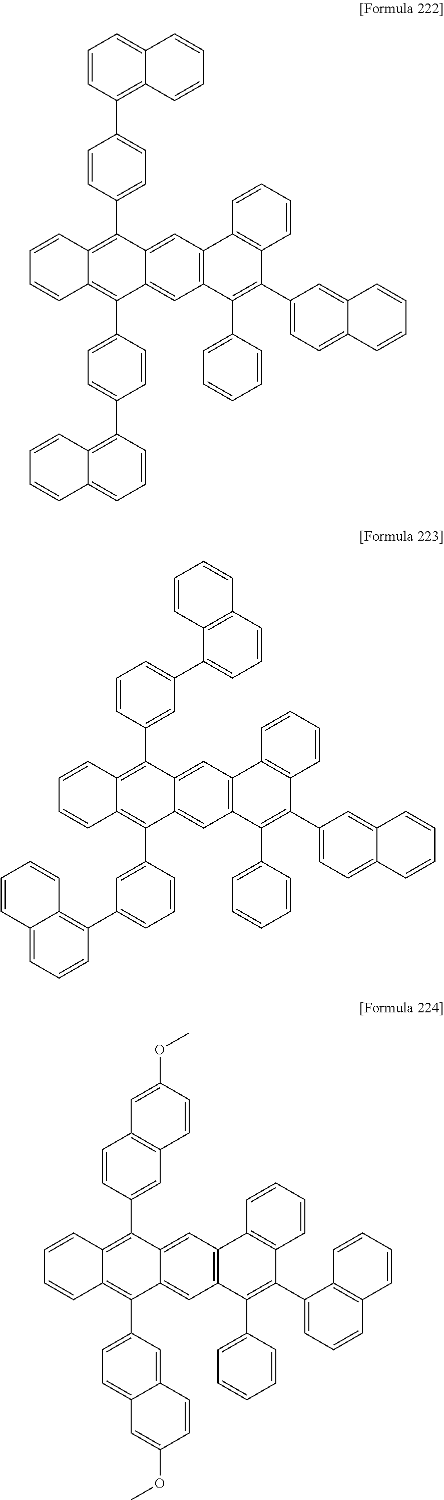

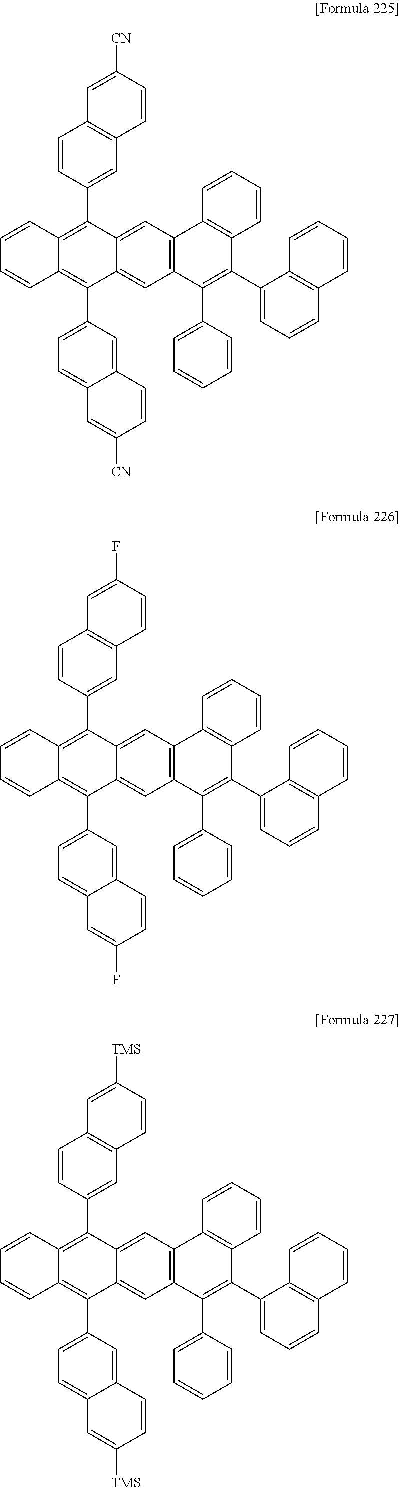

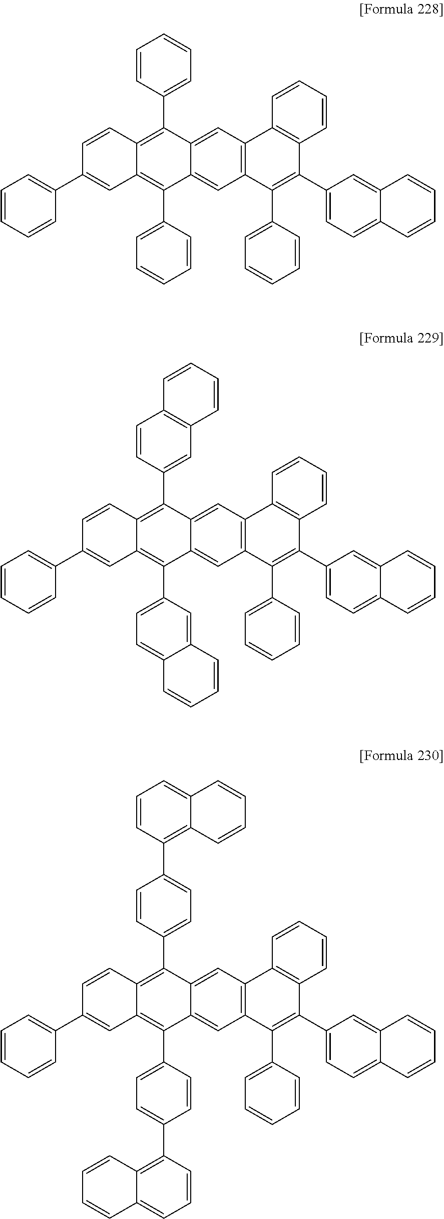

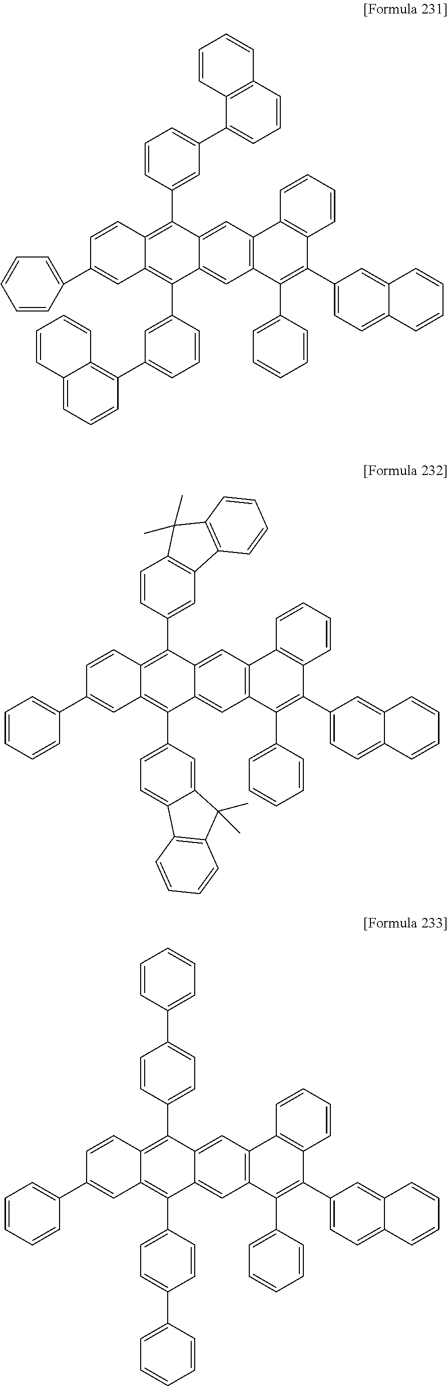

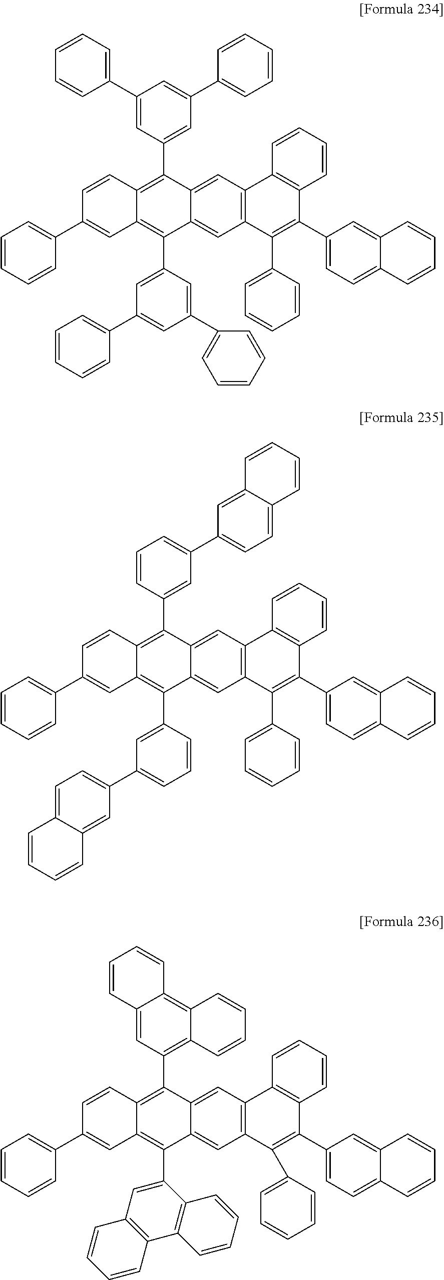

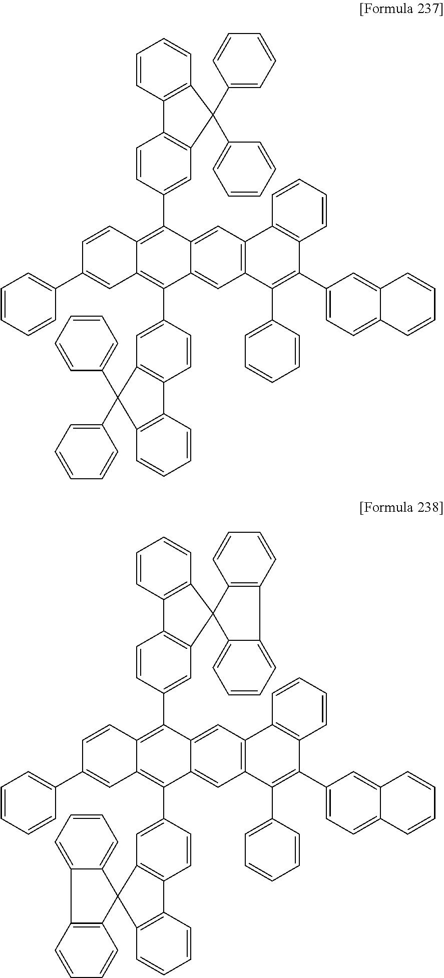

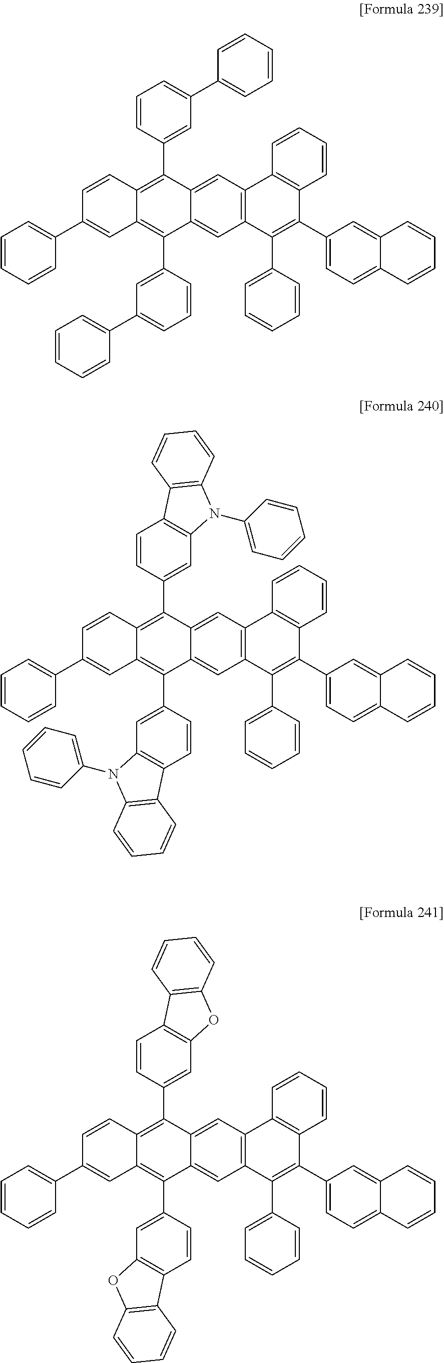

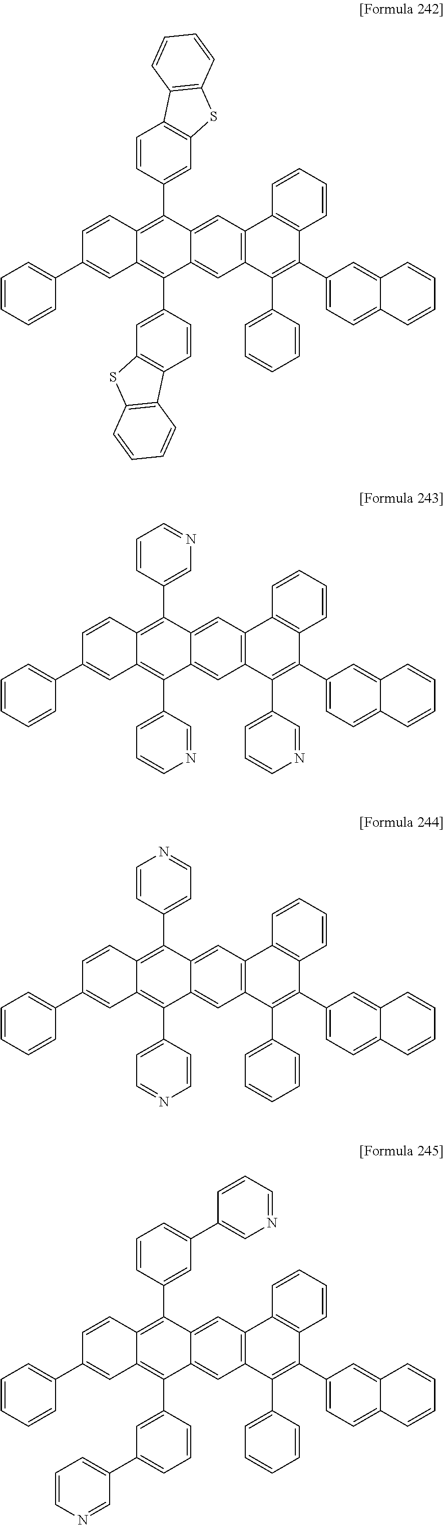

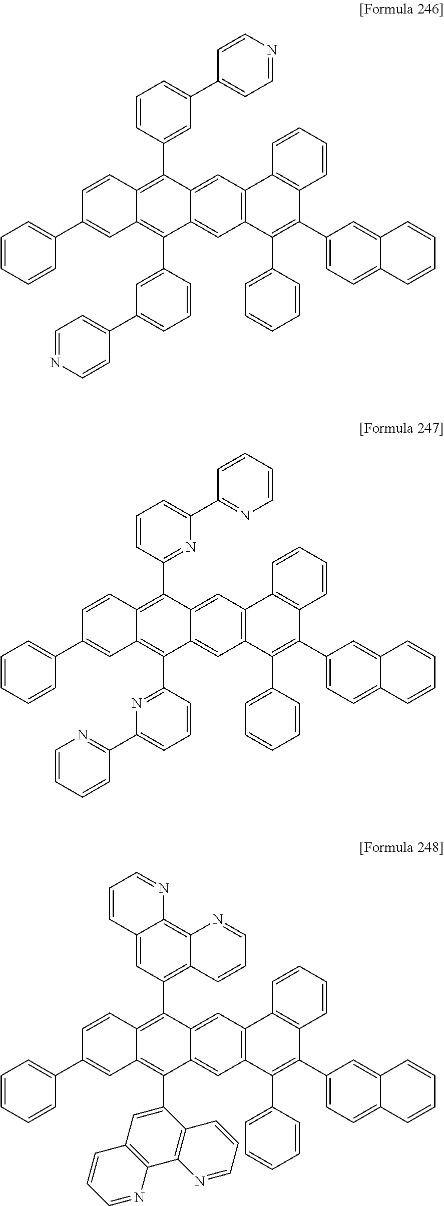

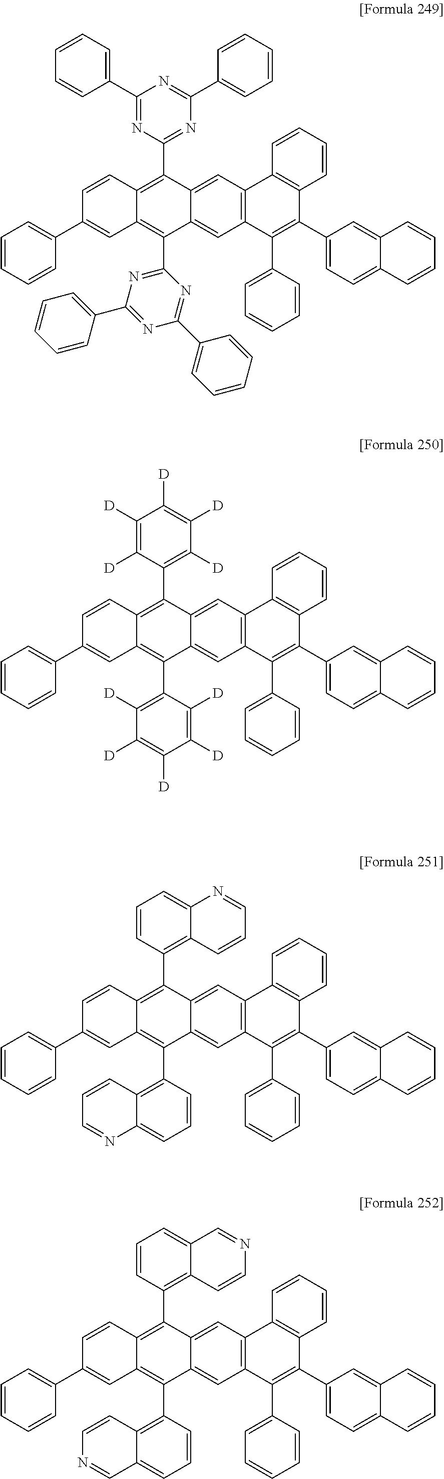

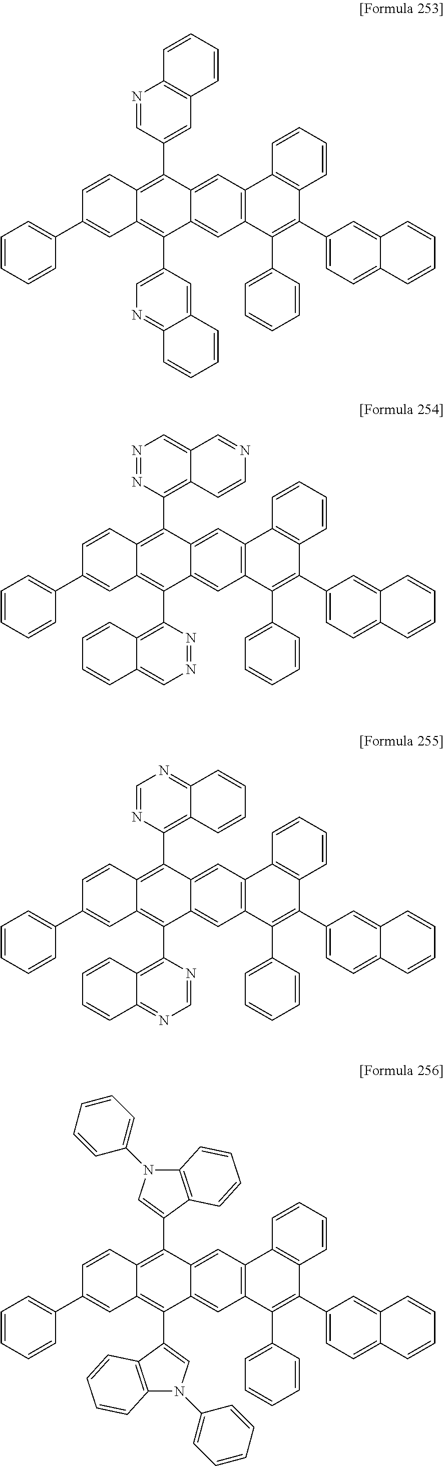

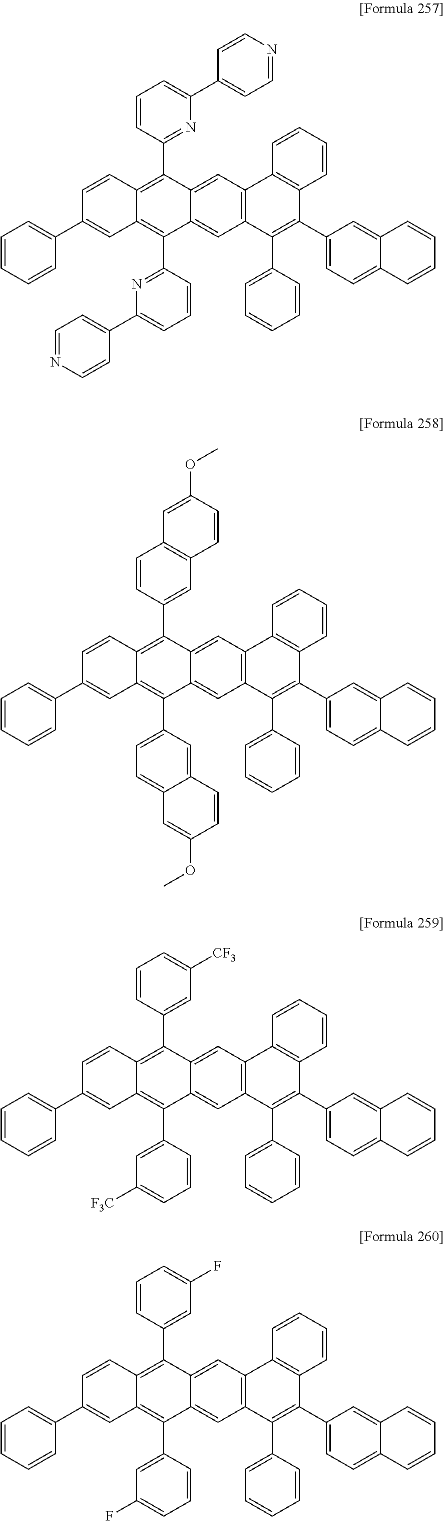

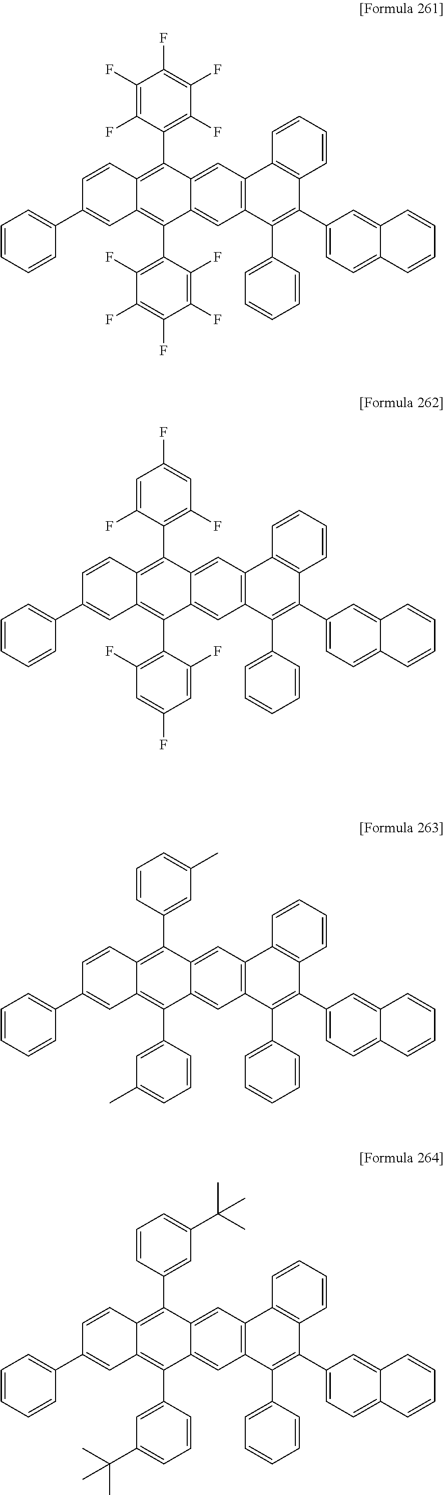

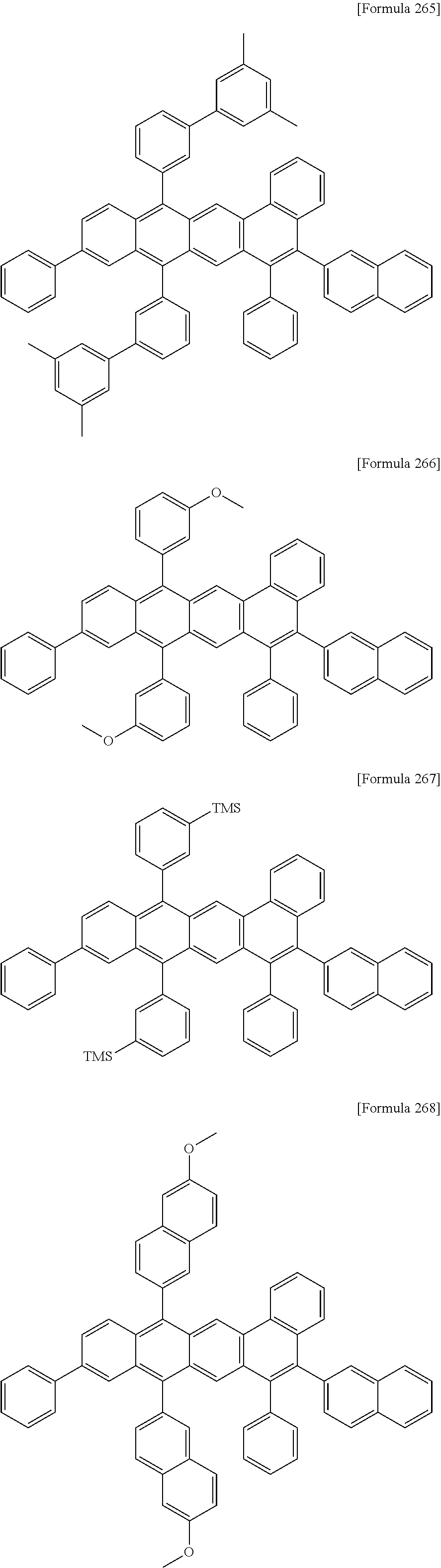

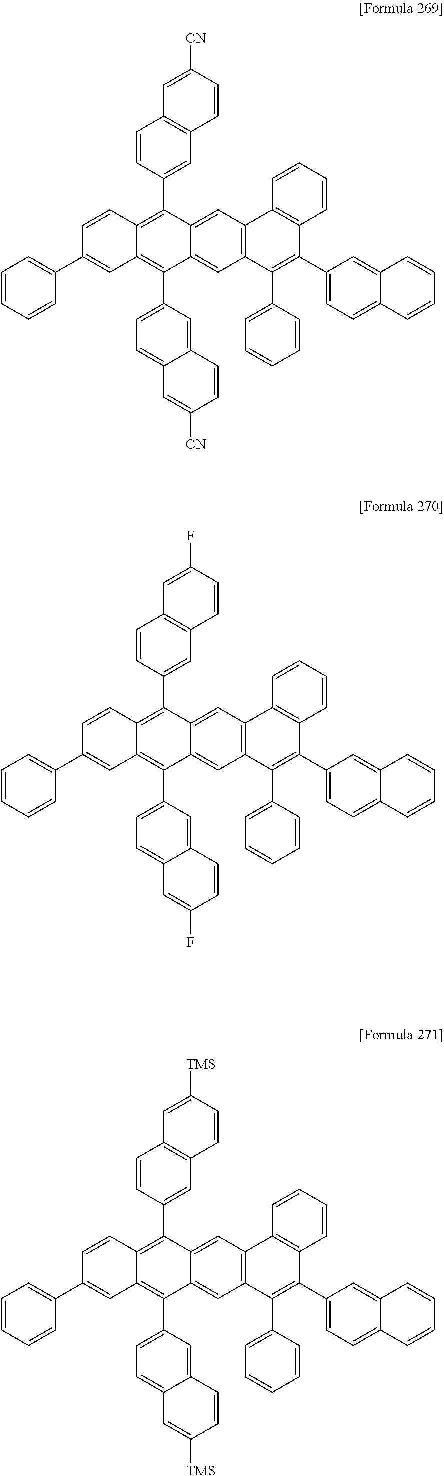

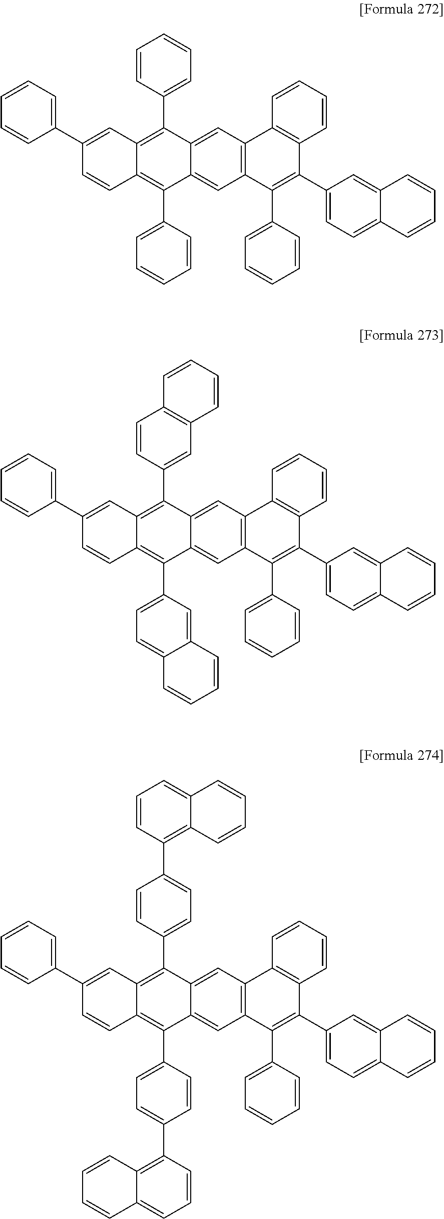

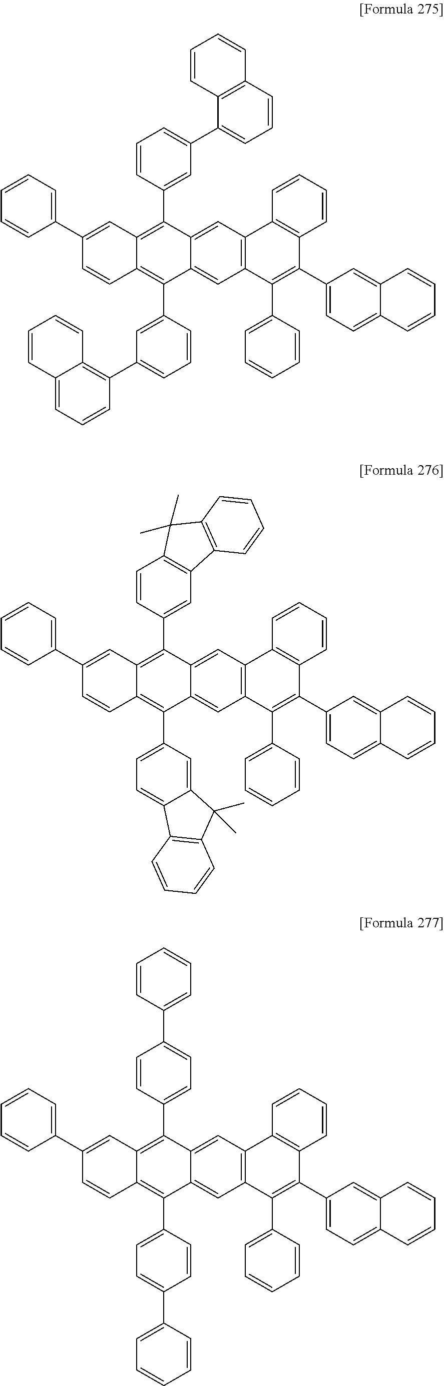

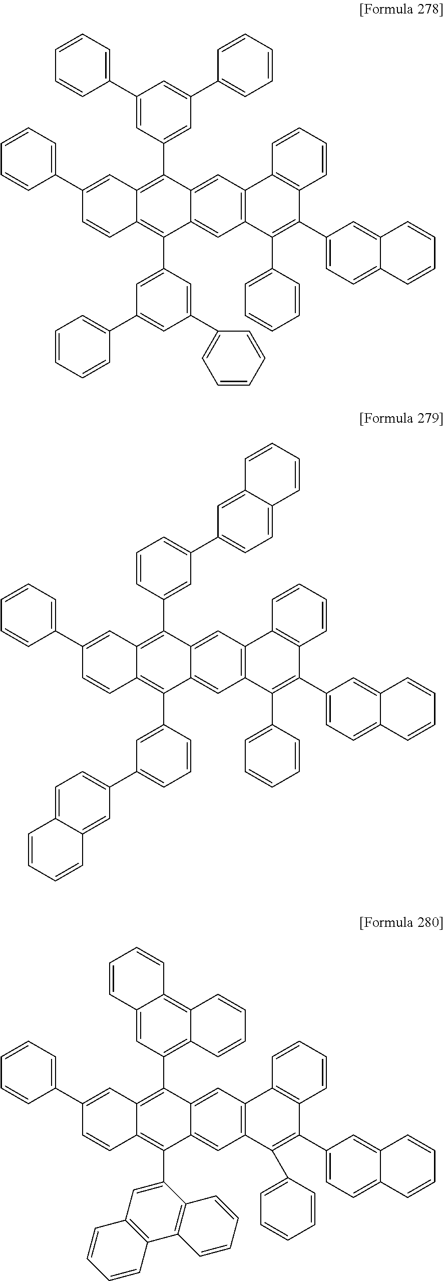

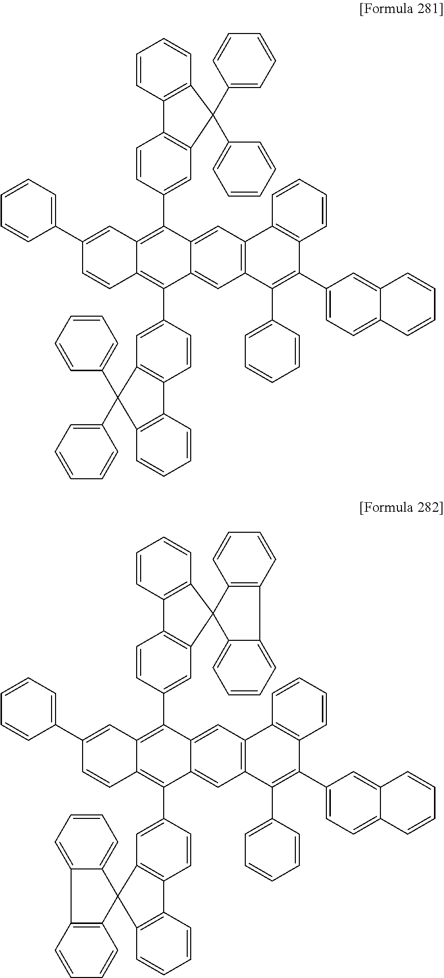

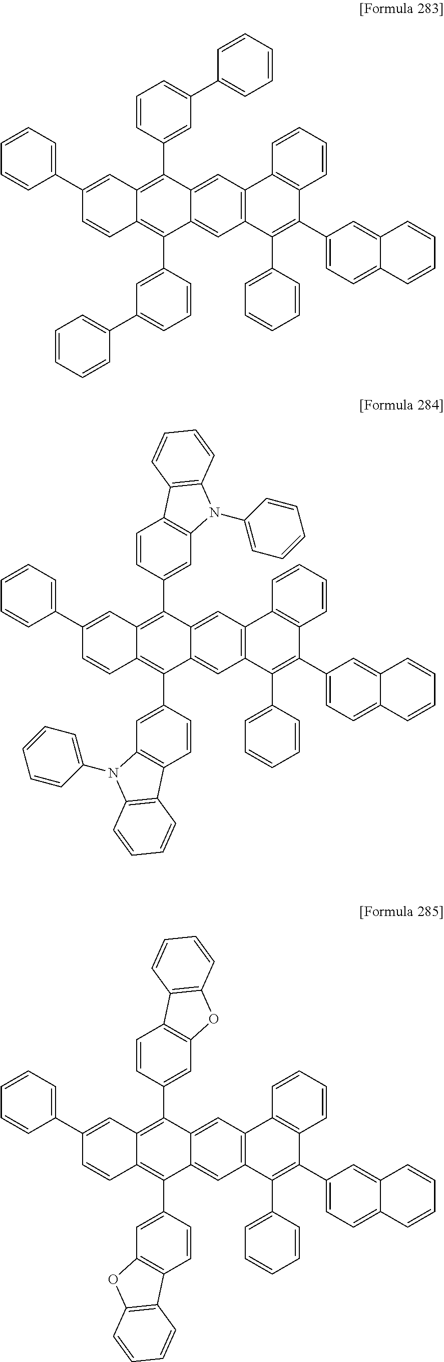

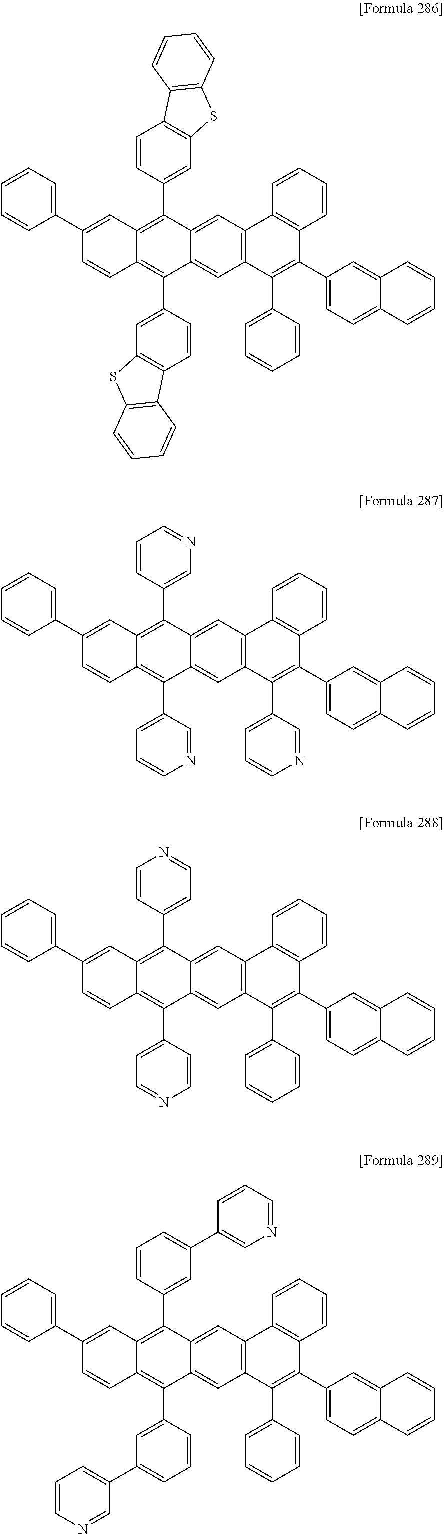

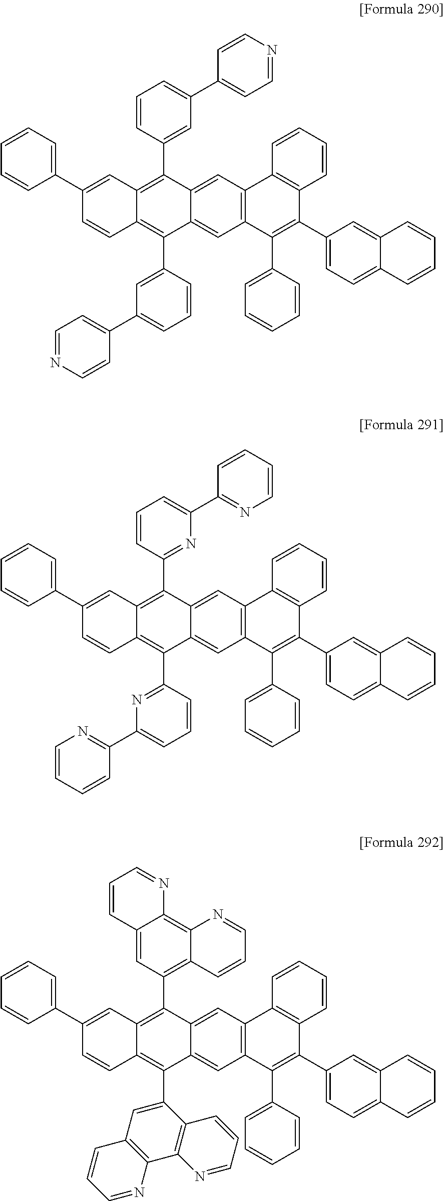

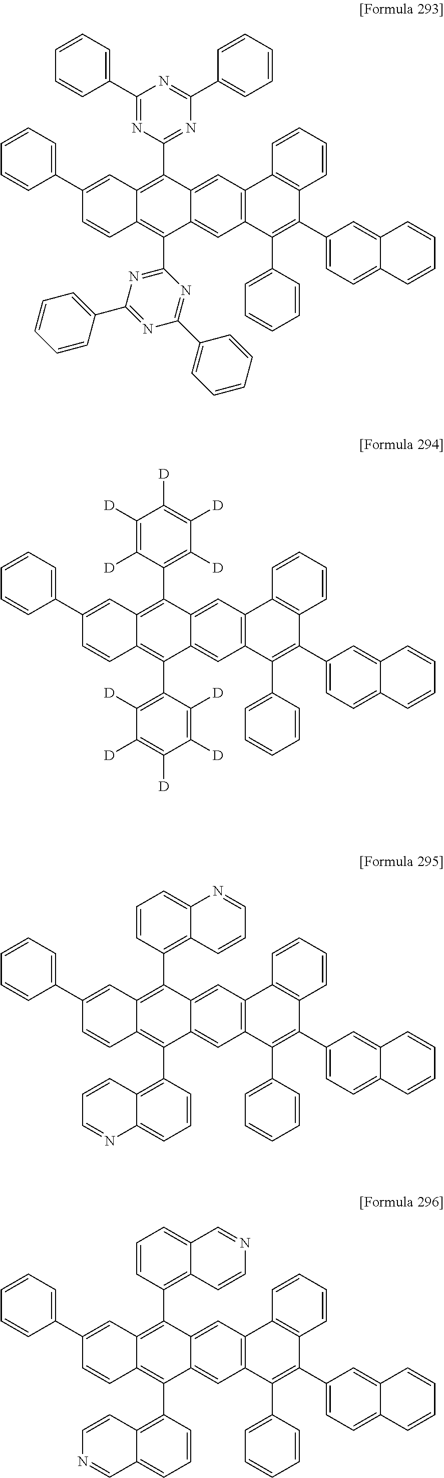

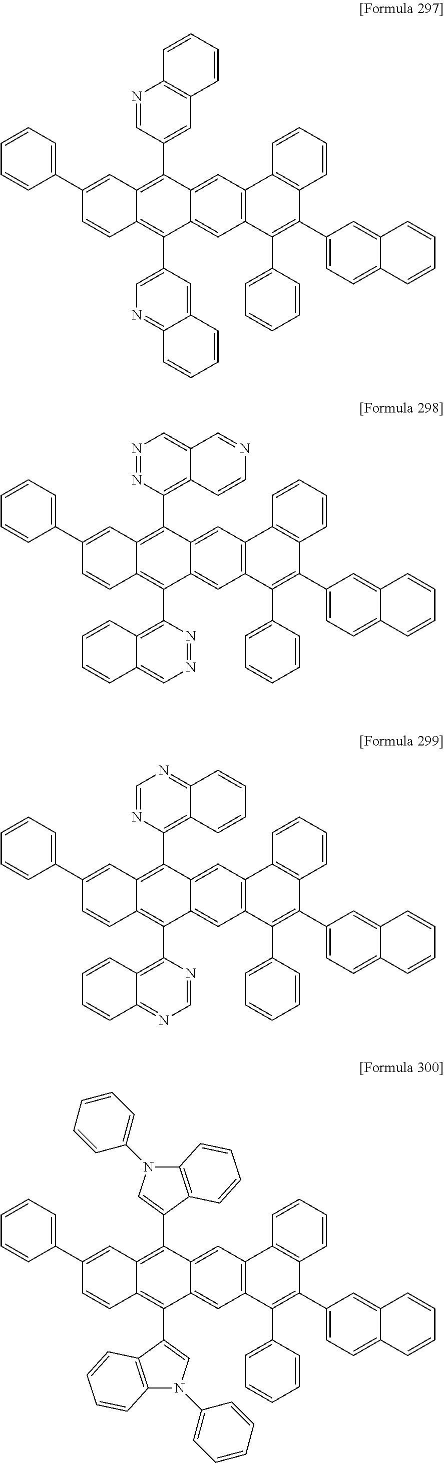

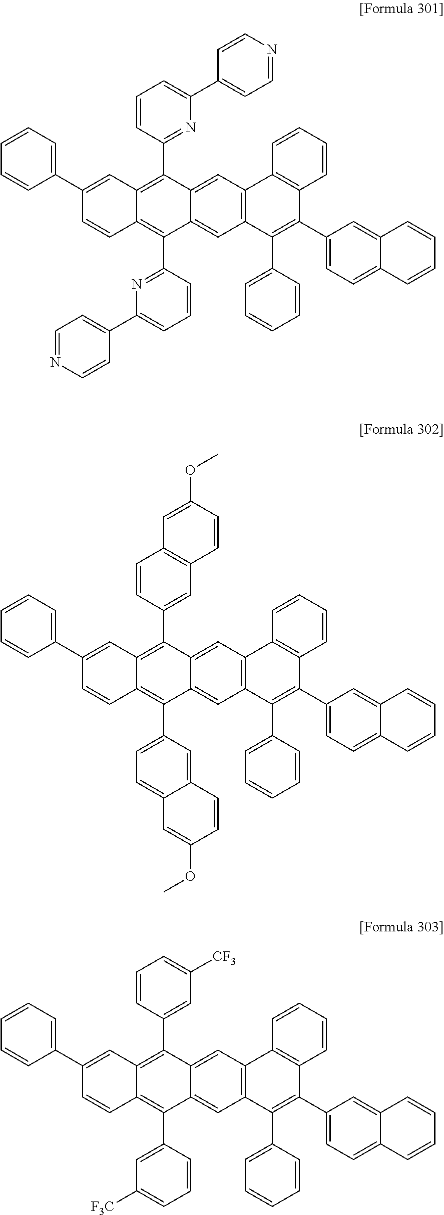

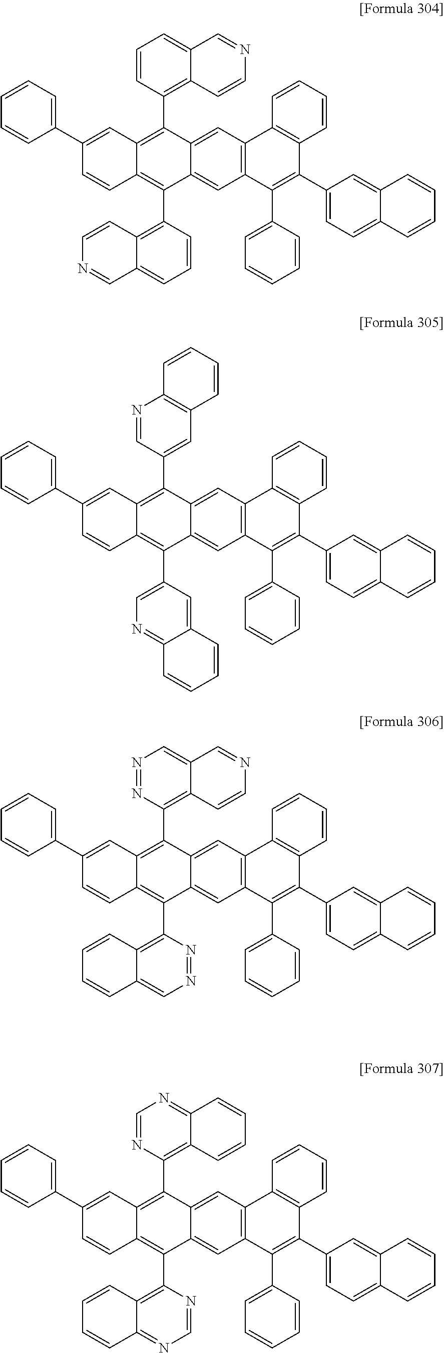

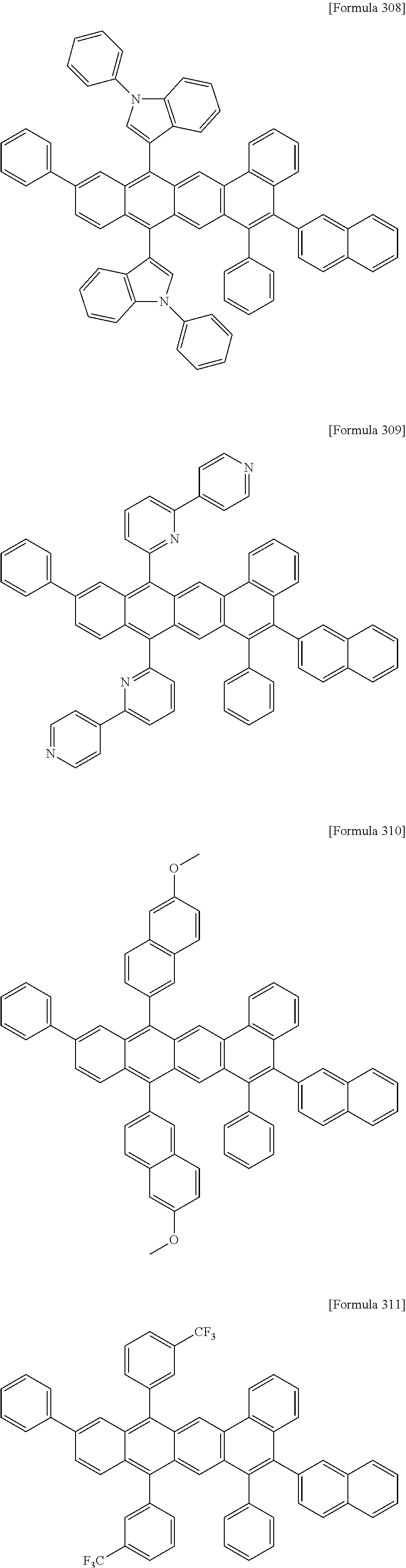

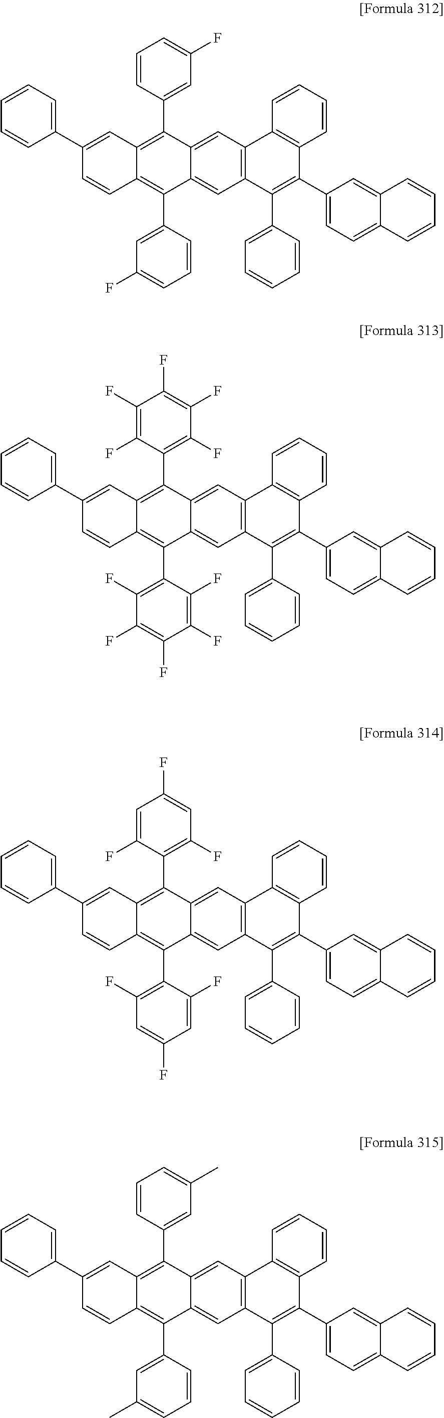

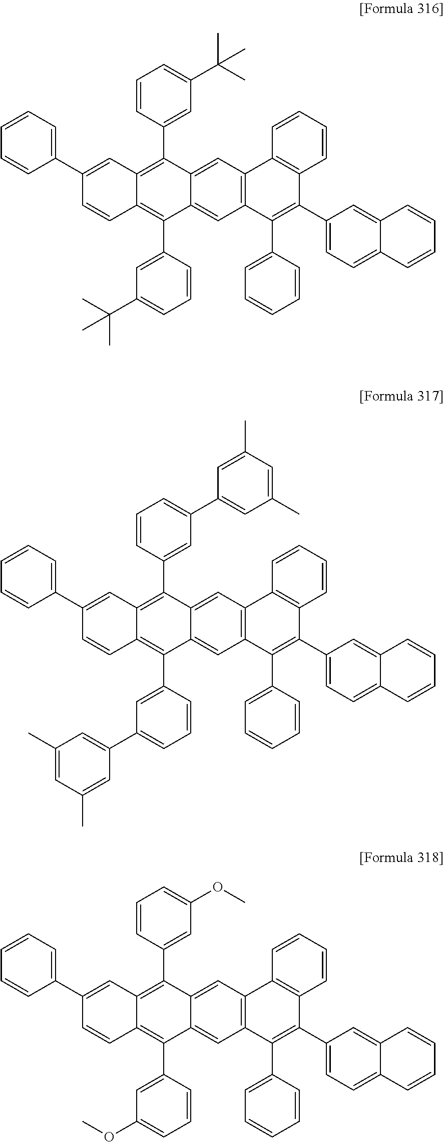

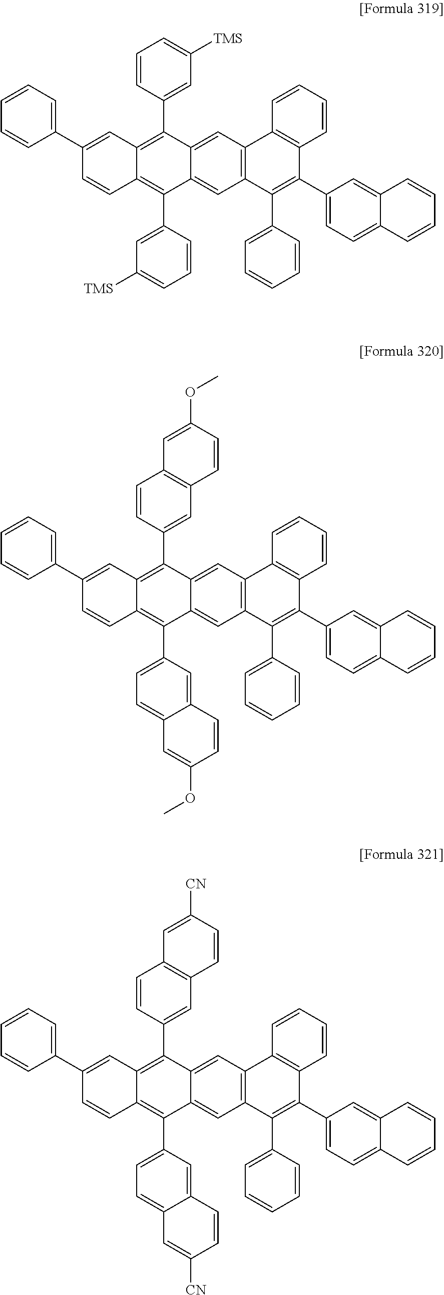

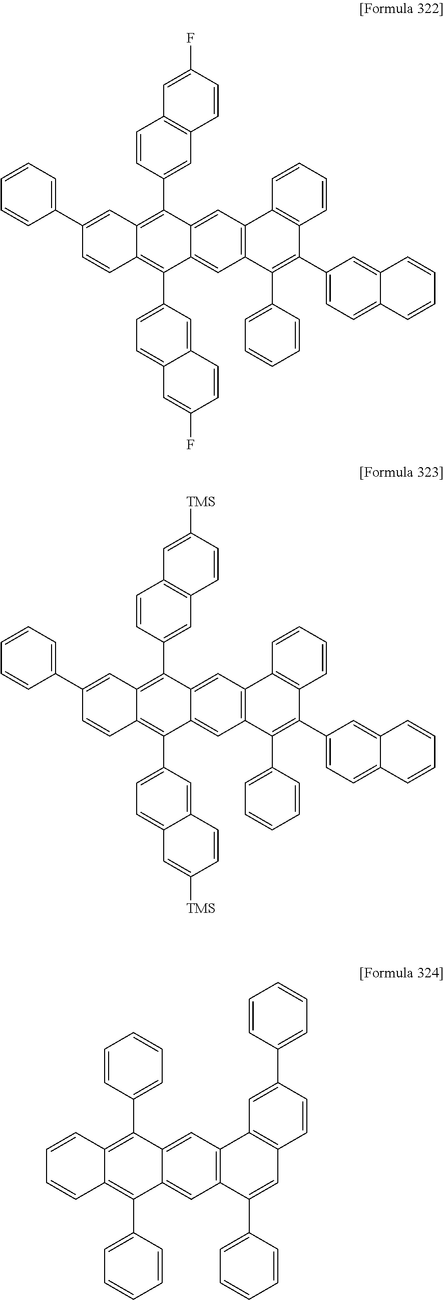

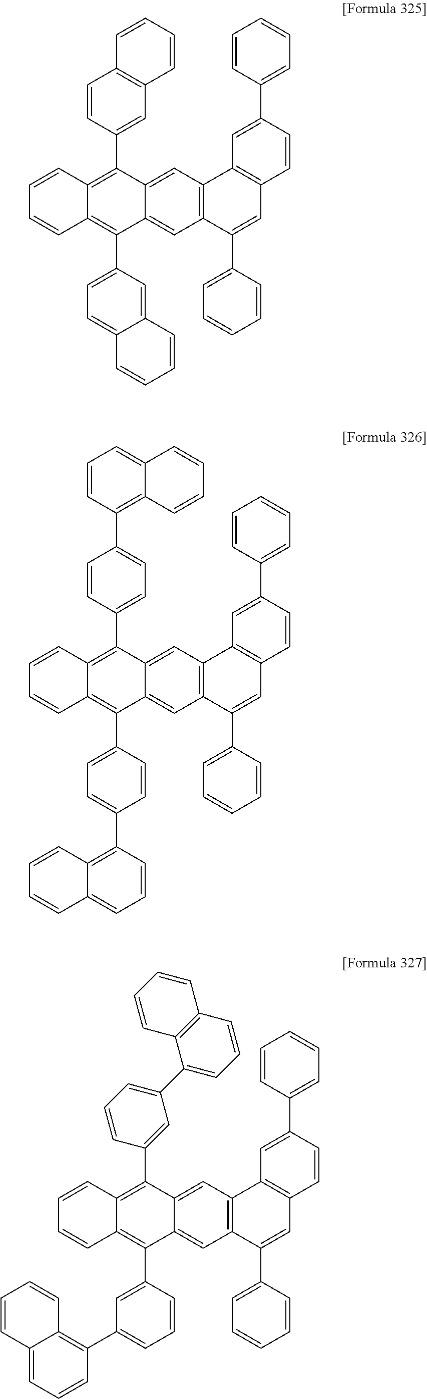

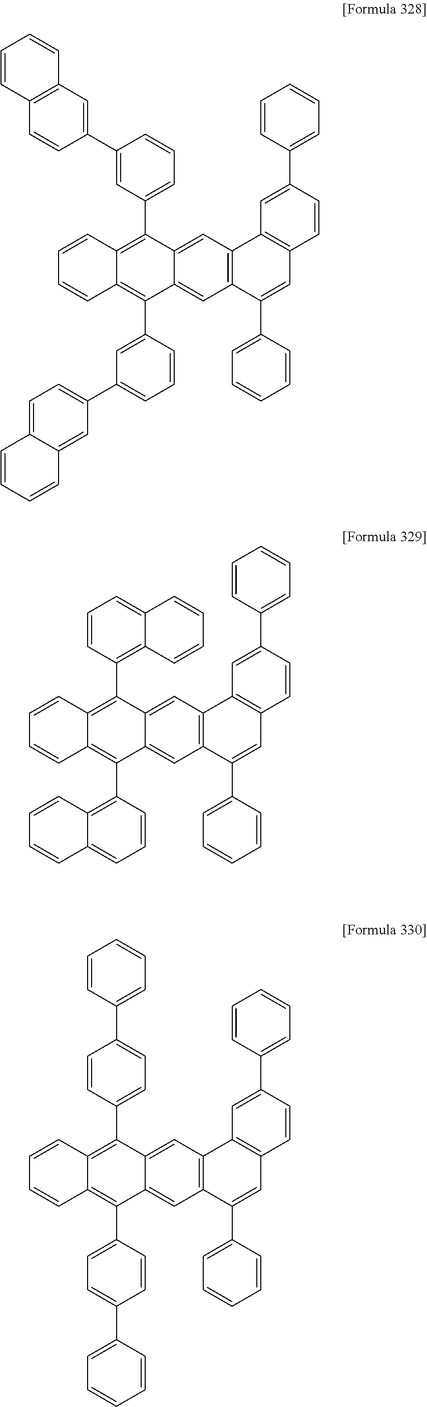

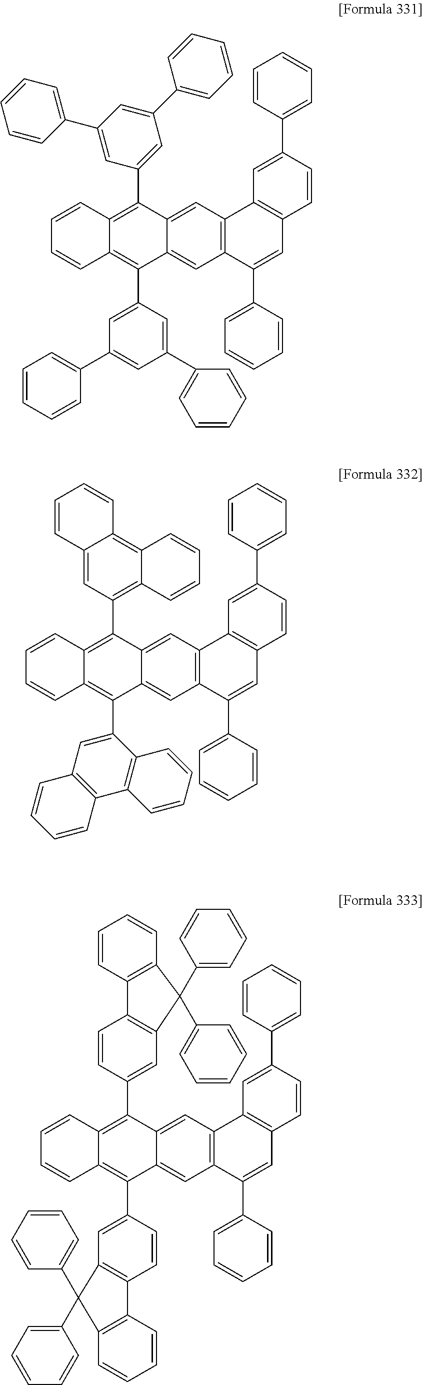

According to another embodiment of the present invention, an organic light-emitting device includes: a first electrode; a second electrode; and an organic layer between the first electrode and the second electrode, where the organic layer includes the compound represented by Formula 1.

The organic layer including the compound represented by Formula 1 may be a hole injection layer, a hole transport layer, a functional layer having hole injection and hole transport capabilities, an electron injection layer, an electron transport layer, or a functional layer having electron transport and electron injection capabilities.

Alternatively, the organic layer may be an emission layer and the compound represented by Formula 1 may be used as a fluorescent or phosphorescent host.

According to an embodiment of the present invention, the organic light-emitting device includes an emission layer, a hole transport layer, and an electron transport layer, and the emission layer may further include an anthracene compound, an arylamine compound, or a styryl compound, which are known compounds.

One or more hydrogen atoms of the anthracene compound, the arylamine compound, or the styryl compound may be substituted with any one of the substituents described above with respect to the C1 to C60 alkyl group. The arylamine refers to a C5 to 60 arylamine group.

An organic light-emitting device according to an embodiment of the present invention may include an emission layer, a hole transport layer, and an electron transport layer, where the emission layer includes a red layer, a green layer, a blue layer, or a white layer, and any one of these layers may further include a phosphorescent compound.

The organic layer of the organic light-emitting device according to an embodiment of the present invention may be a red emission layer. If the organic layer of the organic light-emitting device is a red emission layer, the compound of Formula 1 may be used as a red host.

Also, the first electrode may be an anode and the second electrode may be a cathode. Alternatively, the first electrode may be a cathode and the second electrode may be an anode.

For example, according to an embodiment of the present invention, the organic light-emitting device may have a first electrode/hole injection layer/emission layer/second electrode structure, first electrode/hole injection layer/hole transport layer/emission layer/electron transport layer/second electrode structure, or first electrode/hole injection layer/hole transport layer/emission layer/electron transport layer/electron injection layer/second electrode structure. According to another embodiment of the present invention, the organic light-emitting device may have a first electrode/functional layer having a single film having hole injection and hole transport capabilities/emission layer/electron transport layer/second electrode structure, or a first electrode/functional layer having a single film having hole injection and hole transport capabilities/emission layer/electron transport layer/electron injection layer/second electrode structure. According to another embodiment of the present invention, the organic light-emitting device may have a first electrode/hole transport layer/emission layer/functional layer having a single film having electron injection and electron transport capabilities/second electrode structure, a first electrode/hole injection layer/emission layer/functional layer having a single film having electron injection and electron transport capabilities/second electrode structure, or a first electrode/hole injection layer/hole transport layer/emission layer/functional layer having a single film having electron injection and electron transport capabilities/second electrode structure.

The organic light-emitting device may be a top emission type device, a bottom emission type device, or the like.

The organic layer of the organic light-emitting device may further include a hole injection layer, a hole transport layer, a functional layer having hole injection and hole transport capabilities, an emission layer, a hole blocking layer, an electron transport layer, an electron injection layer, or a combination thereof. However, the structure of the organic layer is not limited to the disclosure above.

At least one of the hole injection layer, the hole transport layer, and the functional layer having hole injection and hole transport capabilities included in the organic light-emitting device may further include a charge-generating material to improve the conductivity of the film, in addition to a known hole injection material, and/or a known hole transport material.

The charge-generating material may be, for example, a p-dopant. Non-limiting examples of the p-dopant include quino derivatives, such as tetracyanoquinodimethane (TCNQ), and 2,3,5,6-tetrafluoro-tetracyano-1,4-benzoquinodimethane (F4TCNQ); metal oxides, such as tungsten oxides and molybdenum oxides; and cyano group-containing compounds, such as Compound 100 below.

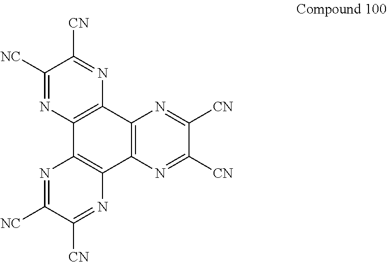

If the hole injection layer, the hole transport layer, or the functional layer having hole injection and hole transport capabilities further includes the charge-generating material, the charge-generating material may be uniformly or non-uniformly dispersed. However, the distribution of the charge-generating material may not be limited to the disclosure above.

The electron transport layer included in the organic light-emitting device may include an electron transporting organic compound and a metal-containing material. Non-limiting examples of the electron transporting organic compound are anthracene compounds, such as 9,10-di(naphthalene-2-yl)anthracene (ADN); and Compounds 101 and 102 below:

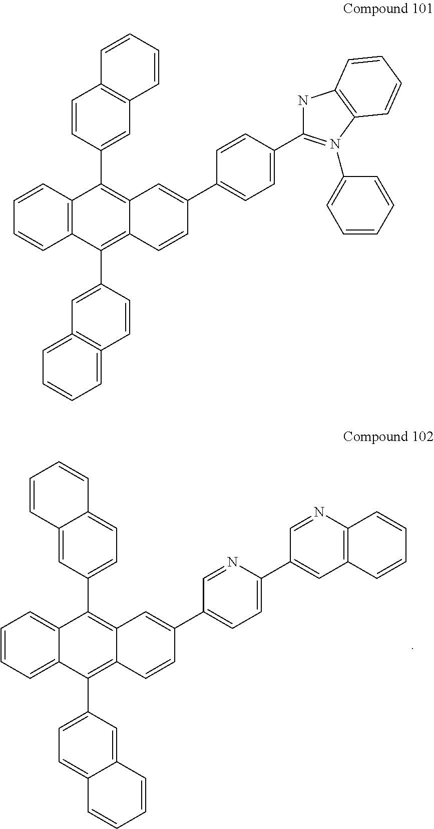

The metal-containing material may include a Li complex. A non-limiting example of the Li complex is lithium quinolate (LiQ) or Compound 103 below:

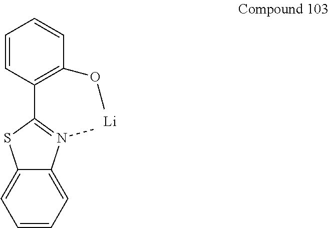

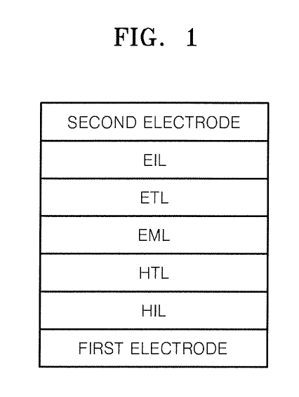

Hereinafter, a method of manufacturing the organic light-emitting device, according to an embodiment of the present invention, is described in detail with reference to FIG. 1. The organic light-emitting device of FIG. 1 includes a substrate, a first electrode (anode), a hole injection layer, a hole transport layer, an emission layer, an electron transport layer, an electron injection layer, and a second electrode (cathode).

First, the first electrode is formed on the substrate by a deposition or sputtering method. The first electrode may be formed of a first electrode material having a high work function. The first electrode may be an anode or a cathode. The substrate may be any substrate conventionally used in organic light-emitting devices, and may include, for example, a glass substrate or a transparent plastic substrate, which have good mechanical strength, thermal stability, transparency, and surface smoothness, are easy to handle and water resistant. An example of the first electrode material is a material having high conductivity, and such a material may be, for example, indium tin oxide (ITO), indium zinc oxide (IZO), tin oxide (SnO2), zinc oxide (ZnO), aluminum (Al), silver (Ag), and magnesium (Mg). The first electrode may be formed as a transparent or reflective electrode.

Then, the hole injection layer (HIL) may be formed on the first electrode by any of a variety of methods, and in some embodiments, by vacuum deposition, spin coating, casting, Langmuir-Blodgett (LB) deposition, or the like.

When the HIL is formed using vacuum deposition, the deposition conditions may vary according to the material that is used to form the HIL, and the structure and thermal characteristics of the HIL. For example, the deposition conditions may include a deposition temperature of about 100 to about 500° C., a vacuum pressure of about 10⁻⁸ to about 10⁻³ torr, and a deposition rate of about 0.01 to about 100 Å/sec.

When the HIL is formed using spin coating, the coating conditions may vary according to the material used to form the HIL, and the structure and thermal properties of the HIL. For example, the coating conditions may include a coating speed of about 2000 rpm to about 5000 rpm, and a thermal treatment temperature of about 80° C. to about 200° C. at which the solvent remaining after coating may be removed.

The material used in forming the HIL may be any one of various known hole injection materials, and non-limiting examples thereof include a phthalocyanine compound such as copperphthalocyanine, 4,4′,4″-tris(3-methylphenylphenylamino) triphenylamine (m-MTDATA), N,N′-di(1-naphthyl)-N,N′-diphenylbenzidine (NPB), TDATA, 2T-NATA, polyaniline/dodecylbenzenesulfonic acid (Pani/DBSA), poly(3,4-ethylenedioxythiophene)/poly(4-styrenesulfonate) (PEDOT/PSS), polyaniline/camphor sulfonic acid (Pani/CSA), and polyaniline/poly(4-styrenesulfonate (PANI/PSS).

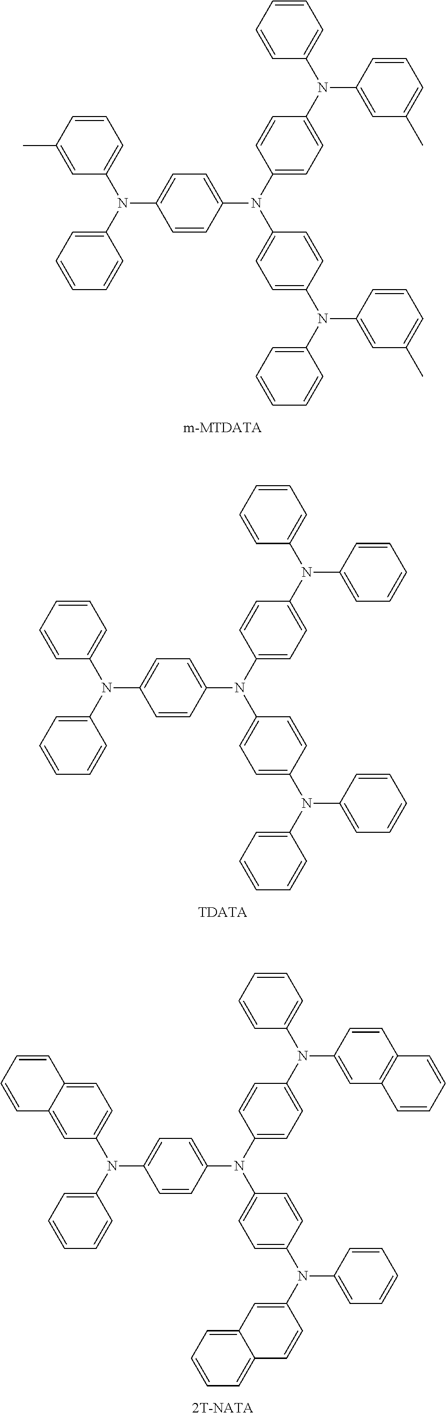

The HIL may have a thickness of about 100 Å to about 10000 Å, and in some embodiments, a thickness of about 100 Å to about 1000 Å. When the thickness of the HIL is within these ranges, the HIL may have good hole injection characteristics without increasing driving voltage.

Next, the hole transport layer (HTL) may be formed on the HIL by any of a variety of methods, and in some embodiments, by vacuum deposition, spin coating, casting, LB deposition, or the like. When the HTL is formed using vacuum deposition or spin coating, the deposition or coating conditions may be similar to those applied to form the HIL, though the deposition or coating conditions may vary according to the material that is used to form the HTL.

The hole transport layer material may be formed using any one of various known hole transport layer materials, and non-limiting examples thereof include carbazole derivatives such as N-phenylcarbazole or polyvinylcarbazole, and amine derivatives having a condensed aromatic ring, such as NPB, N,N′-bis(3-methylphenyl)-N,N′-diphenyl-[1,1-biphenyl]-4,4′-diamine (TPD):

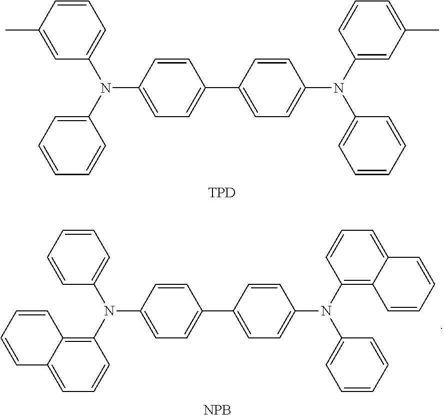

The HTL may have a thickness of about 50 Å to about 1000 Å, and in some embodiments, a thickness of about 100 Å to about 600 Å. When the thickness of the HTL is within these ranges, the HTL may have good hole transport characteristics without substantially increasing driving voltage.

Next, the emission layer (EML) may be formed on the HTL by any of a variety of methods, and in some embodiments, by vacuum deposition, spin coating, casting, LB deposition, or the like. When the EML is formed using vacuum deposition or spin coating, the deposition or coating conditions may be similar to those applied to form the HIL, though the deposition or coating conditions may vary according to the material that is used to form the EML.

The emission layer may include the compound represented by Formula 1. For example, the compound represented by Formula 1 may be used as a host or a dopant. In addition to the compound represented by Formula 1, the EML may be formed using various other known light-emitting materials, for example, a known host and a dopant. In this regard, the dopant may be a known fluorescent dopant or a known phosphorescent dopant.

Non-limiting examples of a known host include Alq₃, 4,4′-N,N′-dicarbazole-biphenyl (CBP), poly(n-vinylcarbazole) (PVK), 9,10-di(naphthalene-2-yl)anthracene (ADN), 1,3,5-tris(N-phenylbenzimidazol-2-yl)benzene (TPBI), E3, and distyrylarylene (DSA).

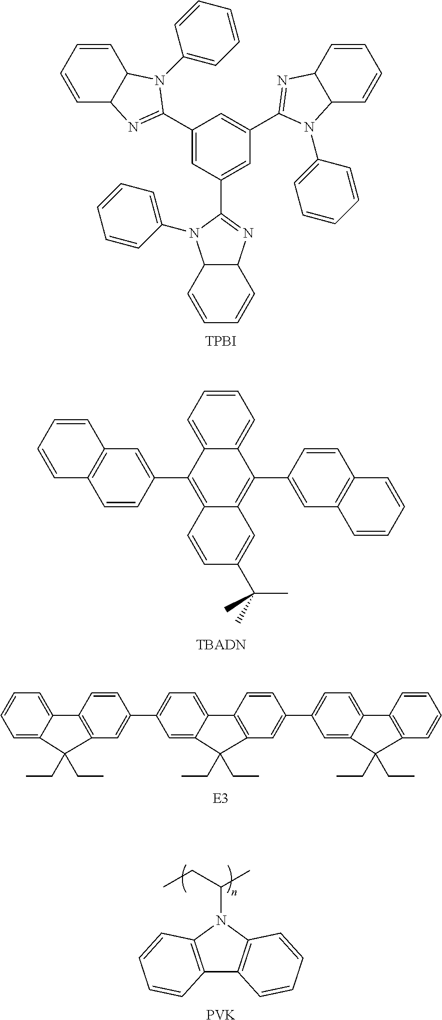

Non-limiting examples of a known red dopant include PtOEP, Ir(piq)₃, Btp₂Ir(acac), and DCJTB.

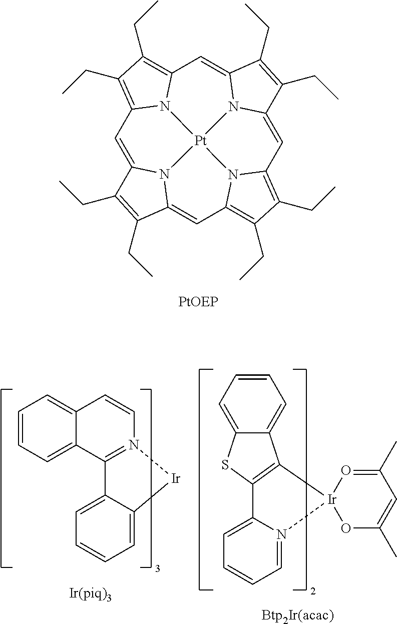

Non-limiting examples of a known green dopant include Ir(ppy)₃ (ppy=phenylpyridine), Ir(ppy)₂(acac), Ir(mpyp)₃, and C545T.

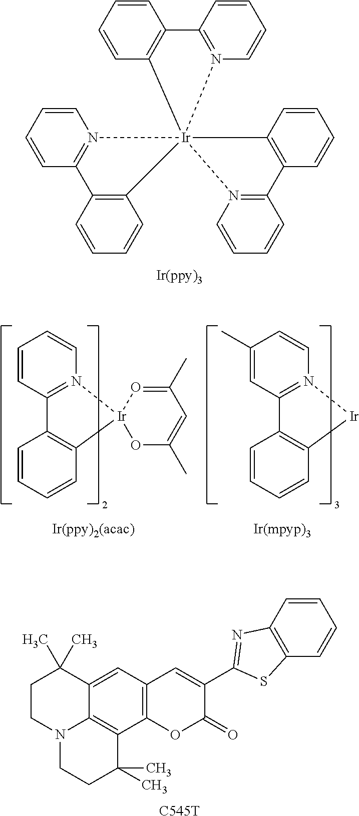

Non-limiting examples of a known blue dopant include F₂Irpic, (F₂ppy)₂Ir(tmd), Ir(dfppz)₃, ter-fluorene, 4,4′-bis(4-diphenylaminostyryl)biphenyl (DPAVBi), and 2,5,8,11-tetra-t-butyl pherylene (TBP).

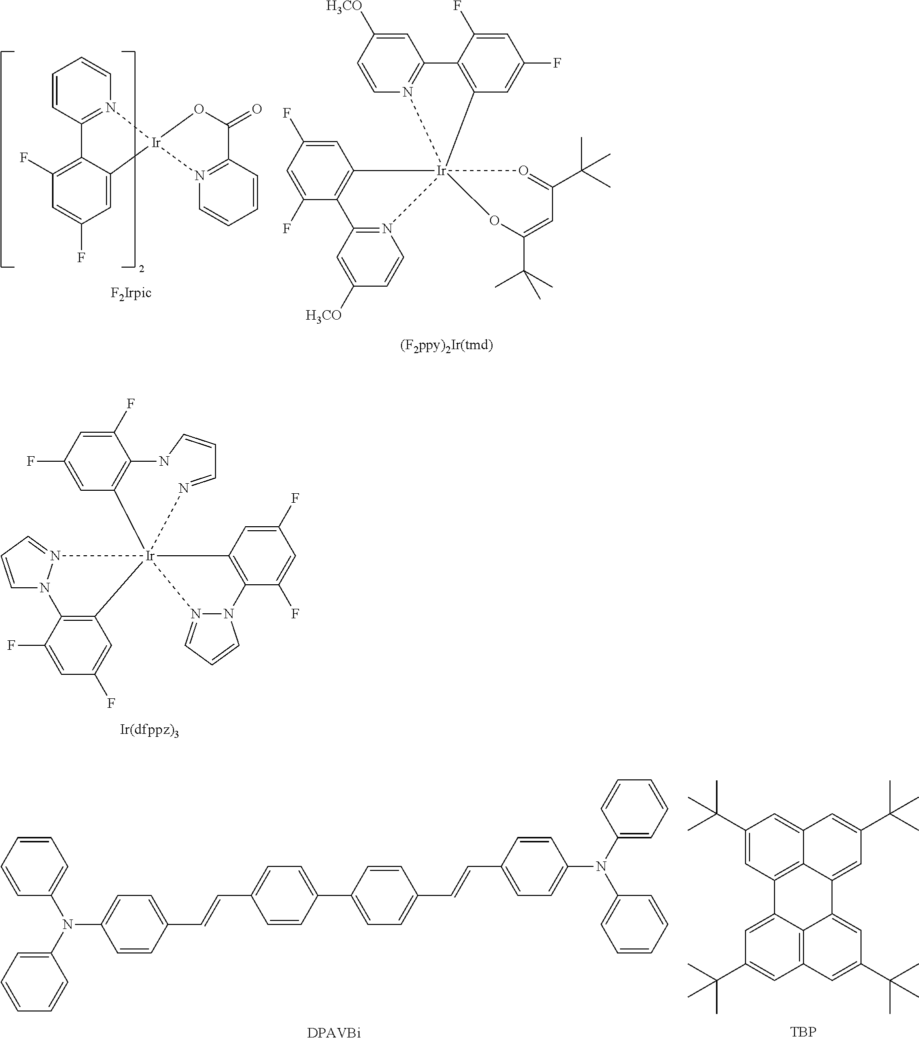

The amount of the dopant may be from about 0.1 to about 20 parts by weight, and in some embodiments, from about 0.5 to about 12 parts by weight, based on 100 parts by weight of the EML material, which is equivalent to the total weight of the host and the dopant. When the amount of the dopant is within these ranges, concentration quenching may be substantially prevented.

The EML may have a thickness of about 100 Å to about 1,000 Å, and in some embodiments, about 200 Å to about 600 Å. When the thickness of the EML is within these ranges, the EML may achieve good luminescent characteristics without substantially increasing driving voltage.

When the EML includes a phosphorescent dopant, a hole blocking layer (HBL) (not shown in FIG. 1) may be formed on the EML in order to prevent diffusion of triplet excitons or holes into the electron transport layer (ETL). In this case, the HBL may be formed of any one of various known materials used to form a HBL. Non-limiting examples of such HBL materials include oxadiazole derivatives, triazole derivatives, phenathroline derivatives, Balq, and BCP.

The HBL may have a thickness of about 50 Å to about 1,000 Å, and in some embodiments, about 100 Å to about 300 Å. When the thickness of the HBL is within these ranges, the HBL may achieve good hole blocking characteristics without substantially increasing driving voltage.

Next, the ETL is formed on the EML (or HBL) by any of a variety of methods, and in some embodiments, by vacuum deposition, spin coating, casting, or the like. When the ETL is formed using vacuum deposition or spin coating, the deposition or coating conditions may be similar to those applied to form the HIL, though the deposition or coating conditions may vary according to the material that is used to form the ETL.

The ETL may be formed using any one of various known materials. Non-limiting examples of the ETL material include quinoline derivatives, such as tris(8-quinolinolate)aluminum (Alq3), TAZ, and BAlq.

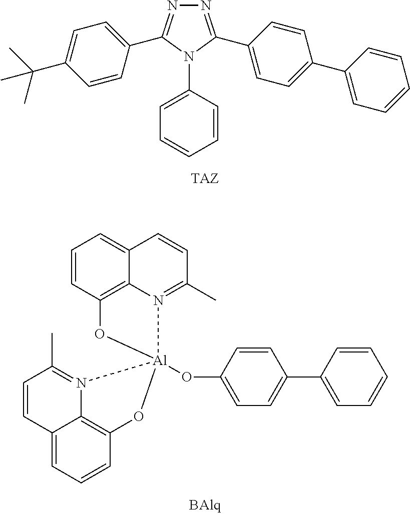

The ETL may have a thickness of about 100 Å to about 1,000 Å, and in some embodiments, about 100 Å to about 500 Å. When the thickness of the ETL is within these ranges, the ETL may achieve good electron transport characteristics without substantially increasing driving voltage.

In addition, the electron injection layer (EIL), which facilitates injection of electrons from the cathode, may be formed on the ETL.

The EIL may be formed using any one of various known materials used to form an EIL, and non-limiting examples thereof include LiF, NaCl, CsF, Li₂O, and BaO. The deposition or coating conditions may be similar to those applied to form the HIL, although the deposition and coating conditions may vary according to the material that is used to form the EIL.

The EIL may have a thickness of about 1 Å to 100 Å, and in some embodiments, about 5 Å to about 90 Å. When the thickness of the EIL is within these ranges, the EIL may achieve good electron injection characteristics without substantially increasing driving voltage.

Finally, the second electrode may be formed on the EIL by, for example, vacuum deposition, sputtering, or the like. The second electrode may be a cathode or an anode. The material for forming the second electrode may include a metal, an alloy, or an electrically conductive compound, which are materials having a low work function, or a mixture thereof. Non-limiting examples of such materials include lithium (Li), magnesium (Mg), aluminum (Al), aluminum-lithium (Al—Li) alloys, calcium (Ca), magnesium-indium (Mg—In) alloys, and magnesium-silver (Mg—Ag) alloys. In addition, in order to manufacture a top-emission organic light-emitting device, a transparent cathode formed of a transparent material such as ITO or IZO may be used as the second electrode.

According to embodiments of the present invention, the organic light-emitting device may be included in various types of flat panel display devices, such as in a passive matrix organic light-emitting display device or in an active matrix organic light-emitting display device. In particular, when the organic light-emitting device is included in an active matrix organic light-emitting display device including a thin-film transistor, the first electrode on the substrate may function as a pixel electrode, and be electrically connected to a source electrode or a drain electrode of the thin-film transistor. Moreover, the organic light-emitting device may also be included in flat panel display devices having double-sided screens.

Also, if the organic layer of the organic light-emitting device includes a plurality of organic layers, one or more layers of the organic layer may be formed by depositing the compound represented by Formula 1 or by a wet process using the compound represented by Formula 1. In the latter case, the compound represented by Formula 1 may be coated on the one or more layers.

Hereinafter, certain embodiments of the present invention will be described in detail with reference to synthesis examples of Compounds 10, 24, 28, 29, 83, 144, 231, and 330 and other examples. However, these examples are presented for illustrative purposes only, and are not intended to limit the scope of the present invention.

### EXAMPLES

### Synthesis Example 1

### Synthesis of Compound 28

a) Synthesis of Formula 1-a

Formula 1-a was synthesized according to Reaction Scheme 1 below.

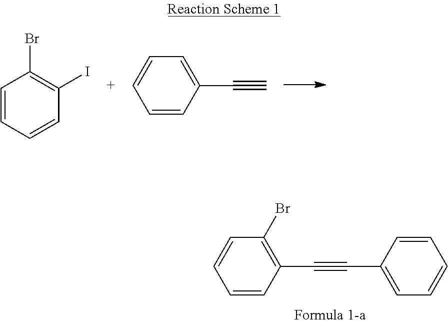

In Reaction Scheme 1, 30.0 g (0.106 mol) of 1-bromo-2-iodobenzene, 1.01 g (0.0005 mol) of copper (I) iodide, 2.45 g (0.002 mol) of Pd(pph₃)₄, and 180 ml of triethylamine were loaded into a 250 ml round bottom flask, followed by stirring. The temperature of the reaction product was decreased to 0° C., and 11.9 g (0.117 mol) of phenyl acetylene was slowly added thereto. After 1 hour of stirring, the reaction was finished. 1 L of normal-hexane was added thereto and the resulting mixture was filtered to remove impurities therefrom, followed by washing. The residual was concentrated and dried to obtain 25.0 g (85.8%) of the compound of Formula 1-a.

b) Synthesis of Formula 1-b

Formula 1-b was synthesized according to Reaction Scheme 2 below.

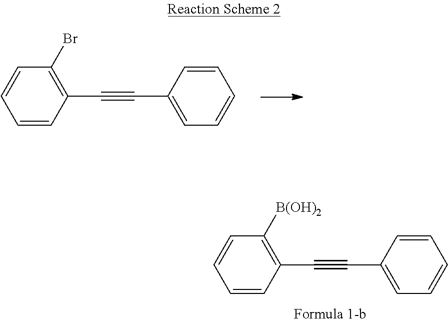

In Reaction Scheme 2, 25.0 g (0.097 mol) of Formula 1-a obtained from Reaction Scheme 1 was dissolved in 250 ml of tetrahydrofuran in a 500 ml round bottom flask and then the mixture was stirred in a nitrogen atmosphere for 30 minutes. Then, the reaction product was decreased to −78° C. and 79 ml of 1.6 M normal butyl lithium in a hexane solution was added thereto dropwise over 30 minutes. At the same temperature, stirring was performed thereon for 2 hours and then 83.3 ml (0.194 mol) of trimethylborate was added dropwise to the resulting mixture over 30 minutes. Then, the temperature was increased to room temperature, followed by 3 hours of stirring. Then, the pH of the mixed solution was controlled with 2N hydrochloric acid solution to be pH 2. The organic layer was separated using ethylether and water, and concentrated under reduced pressure, and then normal-hexane was added thereto, followed by 1 hour of stirring. The stirred solution was filtered to obtain 18.00 g (83.5%) of the compound of Formula 1-b, which was a solid.

c) Synthesis of Formula 1-c

Formula 1-c was synthesized according to Reaction Scheme 3 below.

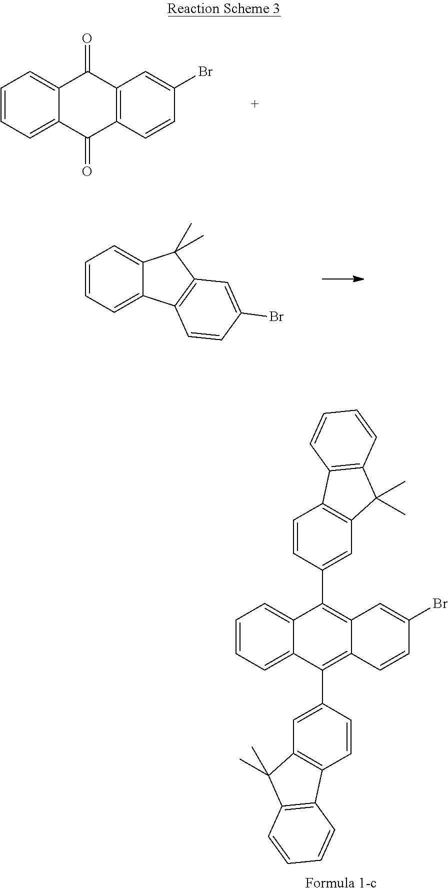

In Reaction Scheme 3, 121.8 g (0.446 mol) of 2-bromo-9,9′-dimethyl fluorene was dissolved in 800 ml of tetrahydrofuran in a 2000 ml round bottom flask and then stirred for 20 minutes in a nitrogen atmosphere. Then, the reaction product was decreased to −78° C., and 263 ml of 1.6 M normal butyl lithium in a hexane solution was added dropwise thereto. At the same temperature, stirring was performed thereon for 2 hours and then 40 g (0.139 mol) of 2-bromo anthraquinone was added to the resulting mixture. After 30 minutes of stirring at the same temperature, the temperature was decreased to room temperature, followed by 3 hours of stirring. Then, the pH of the mixed solution was controlled with 2N hydrochloric acid solution to be pH 2. The organic layer was separated using ethylether and water, and concentrated under reduced pressure and dried. The dried material was dispersed in 600 ml of acetic acid, and 69.38 g (0.418 mol) of potassium iodide and 88.60 g (0.836 mol) of sodium hypophosphite were added thereto and refluxed for 2 hours. The generated solid was filtered under reduced pressure and washed with water and ethanol, followed by recrystallization with toluene. Then, the resulting solid was dried to obtain 64.0 g (71.9%) of the compound of Formula 1-c.

d) Synthesis of Formula 1-d

Formula 1-d was synthesized according to Reaction Scheme 4 below.

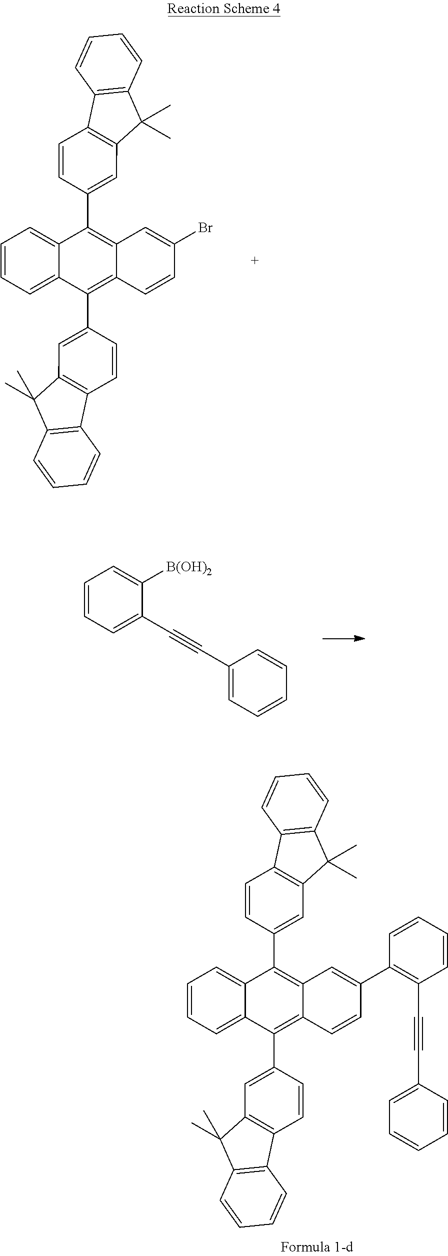

In Reaction Scheme 4, the compound of Formula 1-c obtained from Reaction Scheme 1-3, the compound of Formula 1-b obtained from Reaction Scheme 1-2, 1.44 g (0.001 mol) of Pd(pph₃)₄, and 17.23 g (0.125 mol) of potassium carbonate were loaded into a 500 ml round bottom flask together with 200 ml of toluene, 200 ml of dioxane, and 80 ml of water, followed by 12 hours of refluxing. When the reaction was finished, the reaction product was subjected to reduced pressure to obtain a solid. The organic layer was separated using ethylether and water, and concentrated under reduced pressure, followed by column chromatography using methylene chloride and hexane as an eluent. The obtained solid was dried to produce 25.0 g (54.8%) of the compound of Formula 1-d.

e) Synthesis of Compound 28

Compound 28 was synthesized according to Reaction Scheme 5 below.

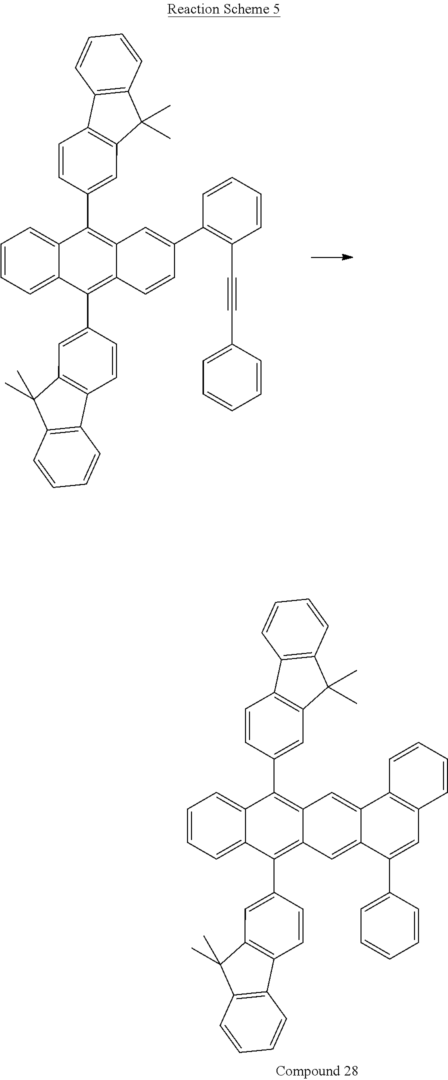

In Reaction Scheme 5, the compound of Formula 1-d obtained from Reaction Scheme 4 was loaded into a 2000 ml round bottom flask and then 800 ml of dichloroethane was added thereto, followed by stirring. 17.0 g (0.034 mol) of iron(II) trifluoromethane sulfonate was added thereto and the mixture was refluxed for 12 hours. When the reaction was finished, a hot methylene chloride was added thereto and subjected to reduced pressure. The residual was concentrated and separated by column chromatography using hexane and methylene chloride as an eluent, followed by recrystallization with tetrahydrofuran. The obtained solid was dried to produce 6.1 g (23.5%) of Compound 28.

MS (MALDI-TOF):m/z=738.33 [M]⁺

EA(Elemental Analysis): calc.—C, 94.27%; H, 5.73%.

(i) found—C, 95.35%; H, 4.65%.

¹H NMR (CDCl₃): δ 8.71 (d, 1H, Ar—H), δ 8.3 (s, 2H, Ar—H), δ 8.12 (d, 1H, Ar—H), δ7.91˜7.69 (m, 10H, Ar—H), δ 7.59˜7.32 (m, 16H, Ar—H), δ 1.59 (d, 12H, —CH₃)

### Synthesis Example 2

### Synthesis of Compound 24

a) Synthesis of Formula 2-a

Formula 2-a was synthesized according to Reaction Scheme 6 below.

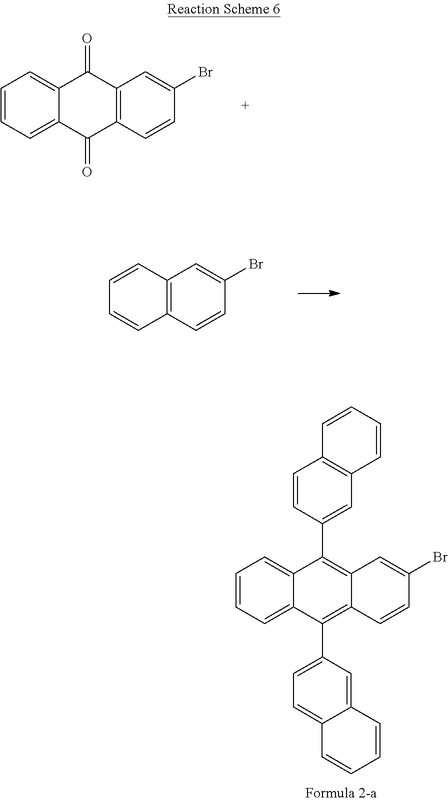

In Reaction Scheme 6, 6.5 g (38.0%) of the compound of Formula 2-a was prepared in the same manner as in Reaction Scheme 3 of Synthesis Example 1, except that 2-bromo naphthalene was used instead of the 2-bromo-9,9′-dimethylfluorene used in Reaction Scheme 3.

b) Synthesis of Formula 2-b

Formula 2-b was synthesized according to Reaction Scheme 7 below.

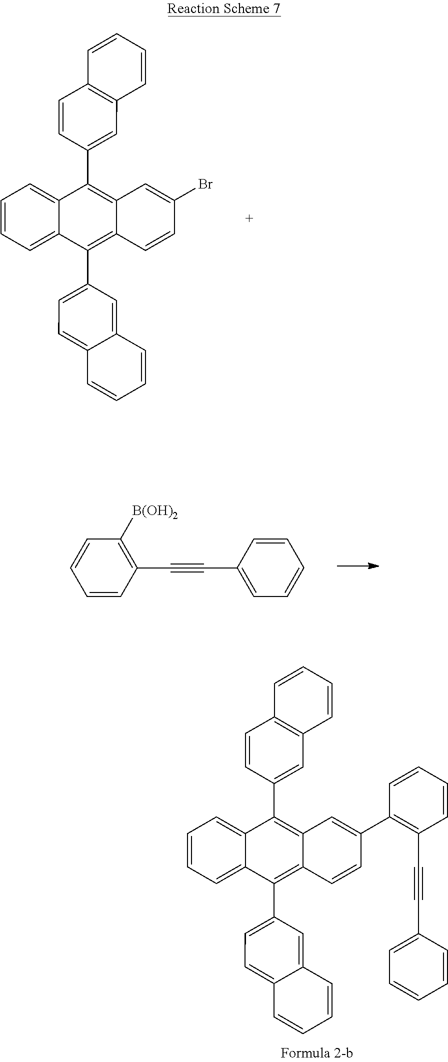

In Reaction Scheme 7, 8.8 g (44.1%) of the compound of Formula 2-b was prepared in the same manner as in Reaction Scheme 4 of Synthesis Example 1, except that the compound of Formula 2-a was used instead of the compound of Formula 1-d used in Reaction Scheme 4.

c) Synthesis of Formula 27

Compound 24 was synthesized according to Reaction Scheme 8 below.

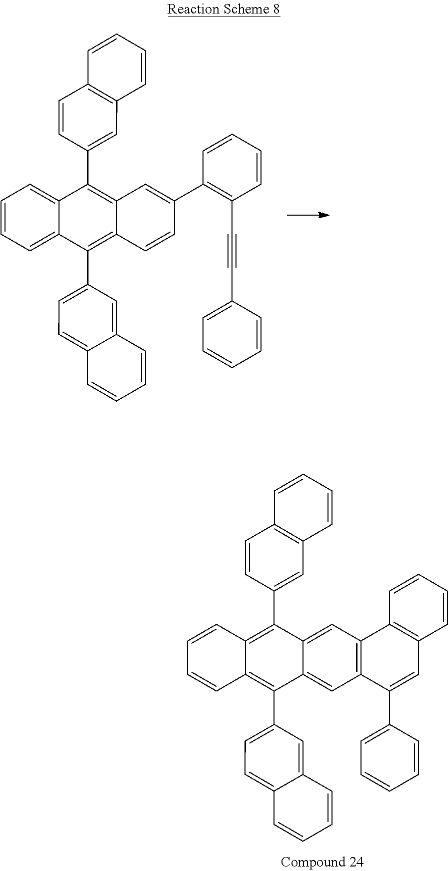

In Reaction Scheme 8, 1.3 g (13.3%) of Compound 24 was prepared in the same manner as in Reaction Scheme 5 of Synthesis Example 1, except that the compound of Formula 2-b was used instead of the compound of Formula 1-d used in Reaction Scheme 5.

MS (MALDI-TOF):m/z=606.23 [M]⁺

EA(Elemental Analysis): calc.—C, 95.02%; H, 4.98%.

(i) found—C, 94.68%; H, 5.32%.

¹H NMR (CDCl₃): δ 8.85 (d, 1H, Ar—H), δ 8.28 (s, 2H, Ar—H), δ 8.11 (d, 1H, Ar—H), δ 7.96˜7.72 (m, 8H, Ar—H), δ 7.61˜7.36 (m, 14H, Ar—H)

### Synthesis Example 3

### Compound 83

a) Synthesis of Formula 3-a

Formula 3-a was synthesized according to Reaction Scheme 9 below.

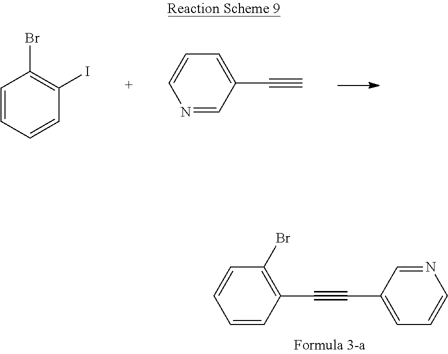

In Reaction Scheme 9, 10.4 g (95.0%) of the compound of Formula 3-a was prepared in the same manner as in Reaction Scheme 1 of Synthesis Example 1, except that 3-etinyl pyridine was used instead of the phenyl acetylene used in Reaction Scheme 1.

b) Formula 3-b

Formula 3-b was synthesized according to Reaction Scheme 10 below.

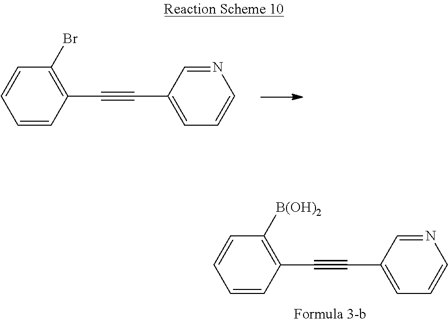

In Reaction Scheme 10, 6.0 g (66.8%) of the compound of Formula 3-b was prepared in the same manner as in Reaction Scheme 2 of Synthesis Example 1, except that the compound of Formula 3-a was used instead of the compound of Formula 2-a used in Reaction Scheme 2.

c) Formula 3-c

Formula 3-c was synthesized according to Reaction Scheme 11 below.

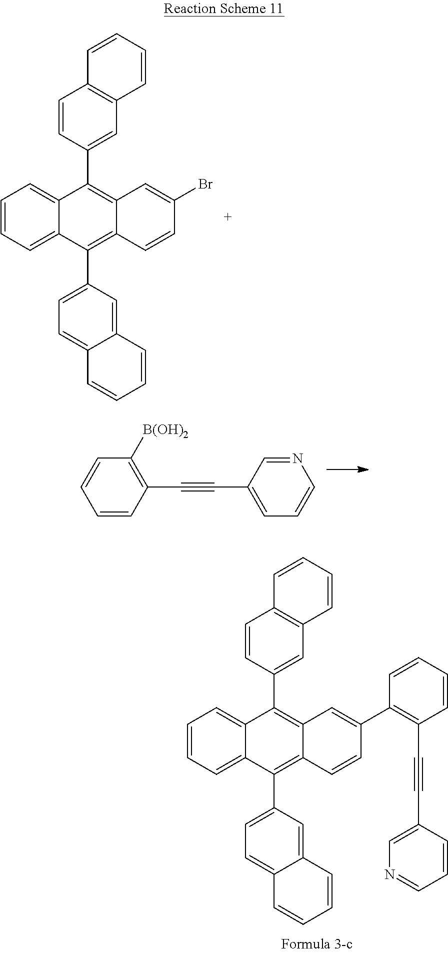

In Reaction Scheme 11, 10.0 g (64.5%) of the compound of Formula 3-c was prepared in the same manner as in Reaction Scheme 4 of Synthesis Example 1, except that the compound of Formula 3-b was used instead of the compound of Formula 1-d used in Reaction Scheme 4.

d) Compound 83

Compound 83 was synthesized according to Reaction Scheme 12 below.

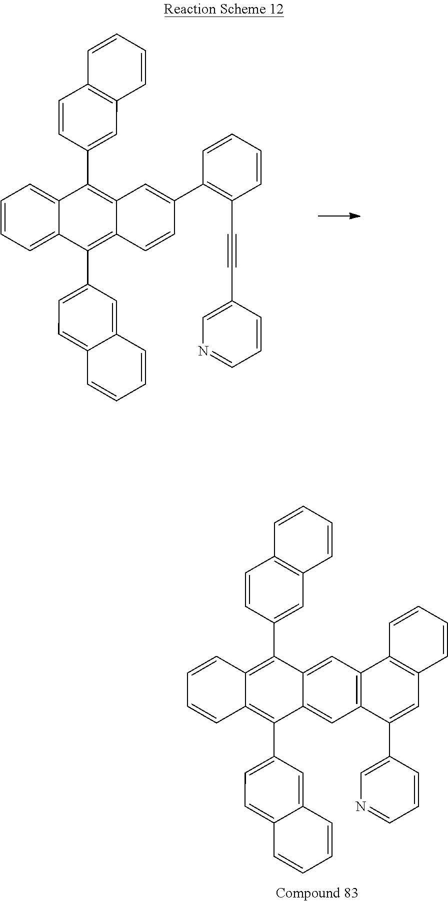

In Reaction Scheme 12, 1.5 g (25.0%) of Compound 83 was prepared in the same manner as in Reaction Scheme 5 of Synthesis Example 1, except that the compound of Formula 3-c was used instead of the compound of Formula 1-d used in Reaction Scheme 5.

MS (MALDI-TOF):m/z=607.23 [M]⁺

EA(Elemental Analysis): calc.—C, 92.89%; H, 4.81%; N, 2.30.

(i) found—C, 93.15%; H, 4.74%; N, 2.11.

1H NMR (CDCl₃): δ 8.92 (s, 1H, Ar—H), δ 8.83 (d, 1H, Ar—H), δ 8.66 (d, 1H, Ar—H), δ 8.30 (s, 2H, Ar—H), δ 8.11 (d, 1H, Ar—H), δ 8.00˜7.80 (m, 10H, Ar—H), δ 7.65˜7.43 (m, 13H, Ar—H)

### Synthesis Example 4

### Synthesis of Compound 330

a) Synthesis of Formula 4-a

Formula 4-a was synthesized according to Reaction Scheme 13 below.

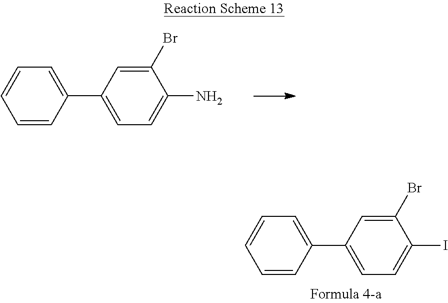

In Reaction Scheme 13, 54.0 g (0.21 mol) of 3-bromo-4-aminobiphenyl and 300 ml of hydrochloric acid were loaded into a 1000 ml round bottom flask and the temperature was decreased to 0° C. 24.4 g (0.21 mol) of a sodium nitrite solution was slowly added dropwise thereto. At the same temperature, stirring was performed thereon for 1 hour and the temperature was increased to 100° C., followed by 1 hour of refluxing. Then, the reaction was finished. An organic material was separated using chloroform and water, and concentrated under reduced pressure. The obtained product was separated by column chromatography using hexane as an eluent, concentrated and dried, and thus, 34.5 g (47.0%) of the compound of Formula 4-a was obtained.

b) Synthesis of Formula 4-b

Formula 4-b was synthesized according to Reaction Scheme 14 below.

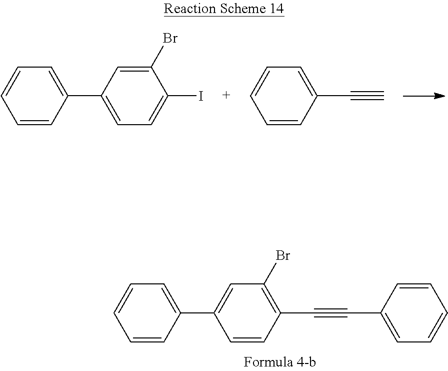

In Reaction Scheme 14, 26.3 g (82.0%) of the compound of Formula 4-b was prepared in the same manner as in Reaction Scheme 1 of Synthesis Example 1, except that 3-bromo-4-iodobiphenyl was used instead of the 1-bromo-2-iodobenzene used in Reaction Scheme 1.

c) Synthesis of Formula 4-c

Formula 4-c was synthesized according to Reaction Scheme 15 below.

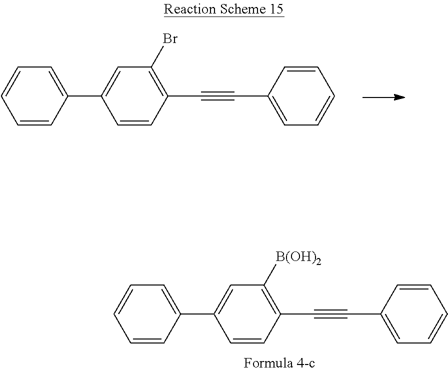

In Reaction Scheme 15, 12.1 g, (51.3%) of the compound of Formula 4-c was prepared in the same manner as in Reaction Scheme 2 of Synthesis Example 1, except that the compound of Formula 4-b was used instead of the compound of Formula 2-a used in Reaction Scheme 2.

d) Synthesis of Formula 4-d

Formula 4-d was synthesized according to Reaction Scheme 16 below.

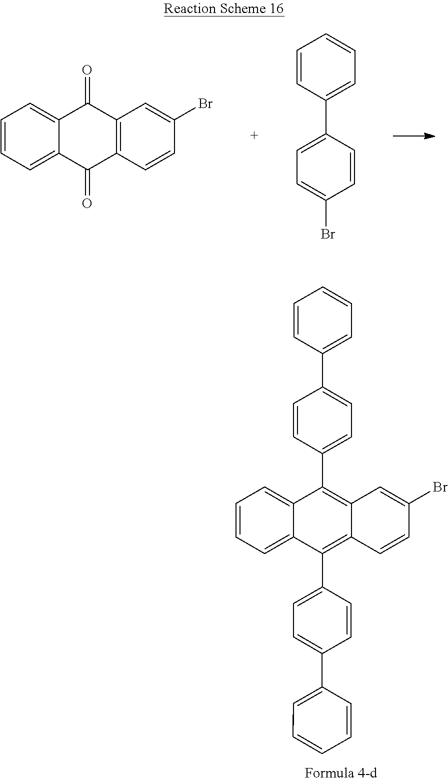

In Reaction Scheme 16, 36.2 g (60.0%) of the compound of Formula 4-d was prepared in the same manner as in Reaction Scheme 3 of Synthesis Example 1, except that 4-bromo biphenyl was used instead of the 2-bromo-9,9-dimethylfluorene used in Reaction Scheme 3.

e) Synthesis of Formula 4-e

Formula 4-e was synthesized according to Reaction Scheme 17 below.

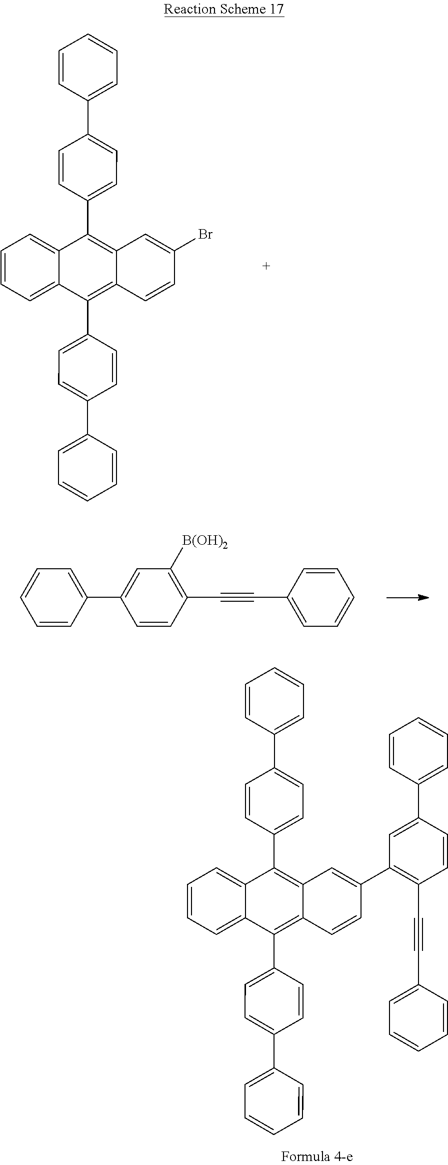

In Reaction Scheme 17, 16.1 g, (34.1%) of the compound of Formula 4-e was prepared in the same manner as in Reaction Scheme 4 of Synthesis Example 1, except that the compound of Formula 4-c was used instead of the compound of Formula 1-b used in Reaction Scheme 4.

f) Synthesis of Compound 330

Compound 330 was synthesized according to Reaction Scheme 18 below.

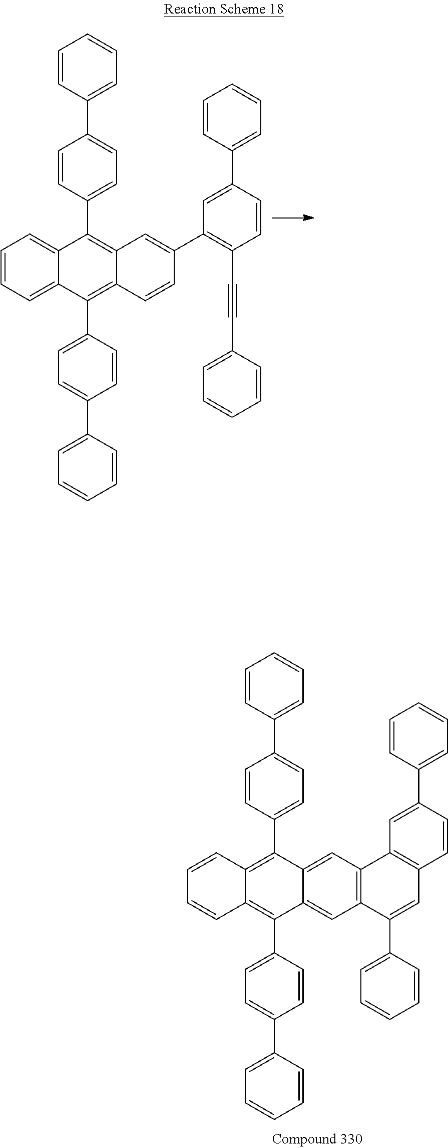

In Reaction Scheme 18, 3.38 g (21.0%) of Compound 330 was prepared in the same manner as in Reaction Scheme 5 of Synthesis Example 1, except that the compound of Formula 4-e was used instead of the compound of Formula 1-d used in Reaction Scheme 5.

MS (MALDI-TOF):m/z=734.30 [M]⁺

EA(Elemental Analysis): calc.—C, 94.79%; H, 5.21%.

(i) found—C, 95.24%; H, 4.76%.

¹H NMR (CDCl₃): δ 8.79 (d, 1H, Ar—H), δ 8.31 (s, 2H, Ar—H), δ 8.20 (d, 1H, Ar—H), δ 8.04 (d, 1H, Ar—H), δ 7.88˜7.70 (m, 12H, Ar—H), δ 7.56˜7.21 (m, 21H, Ar—H)

### Synthesis Example 5

### Synthesis of Compound 29

a) Synthesis of 5-a

Formula 5-a was synthesized according to Reaction Scheme 19 below.

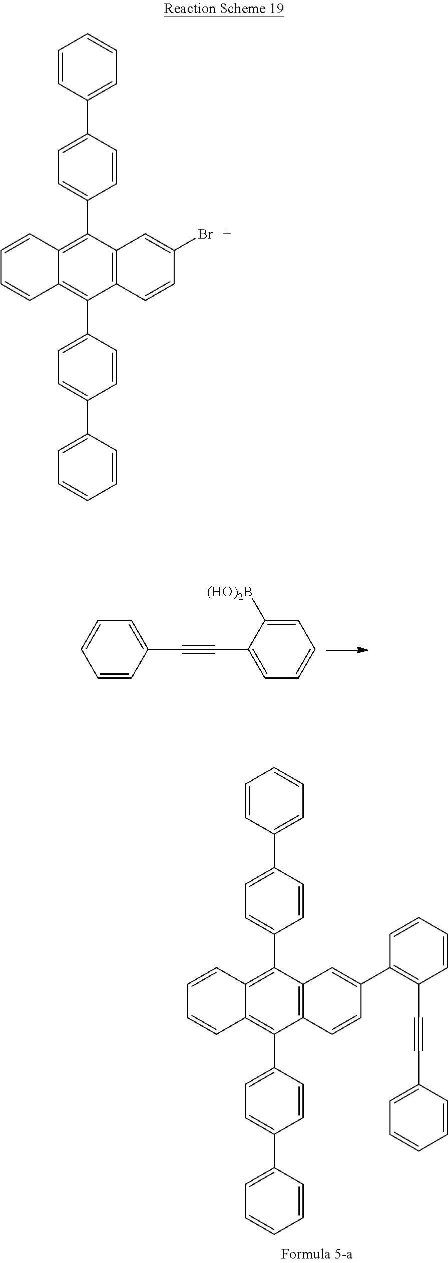

In Reaction Scheme 19, 16 g (57.8%) of the compound of Formula 5-a was prepared in the same manner as in Reaction Scheme 4 of Synthesis Example 1, except that 9,10-biphenyl-2-bromo anthracene was used instead of the compound of Formula 1-c used in Reaction Scheme 4.

b) Synthesis of 5-b

Compound 29 was synthesized according to Reaction Scheme 20 below.

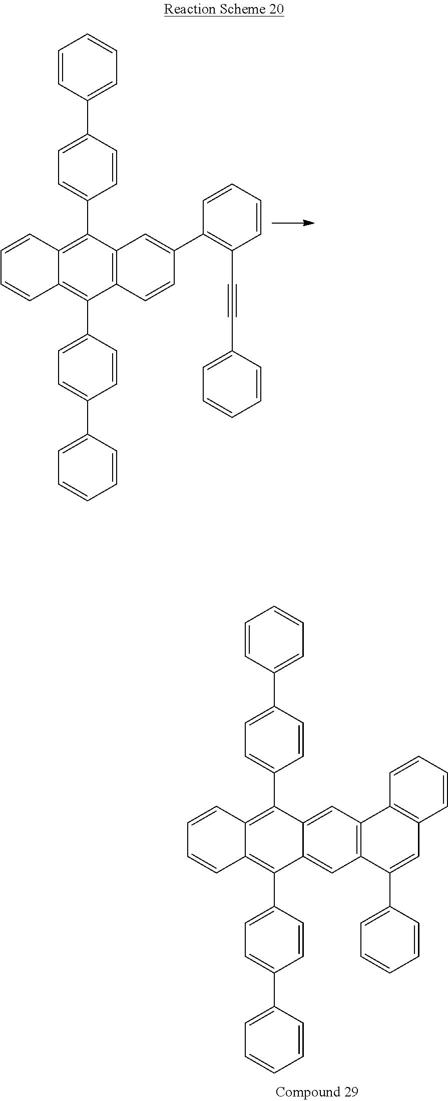

In Reaction Scheme 20, 4.5 g (13.3%) of Compound 29 was prepared in the same manner as in Reaction Scheme 5 of Synthesis Example 1, except that the compound of Formula 5-a was used instead of the compound of Formula 1-d used in Reaction Scheme 5.

MS (MALDI-TOF):m/z=658.27[M]⁺

EA(Elemental Analysis): calc.—C, 94.80% H, 5.20%.

(i) found—C, 94.99%; H, 5.01%.

¹H NMR (CDCl₃): δ 8.74 (d, 1H, Ar—H), b 8.32 (s, 2H, Ar—H), δ 8.16 (d, 1H, Ar—H), δ 7.94˜7.76 (m, 12H, Ar—H), δ 7.54˜7.21 (m, 18H, Ar—H)

### Synthesis Example 6

### Synthesis of Compound 10

a) Synthesis of 6-a

Formula 6-a was synthesized according to Reaction Scheme 21 below.

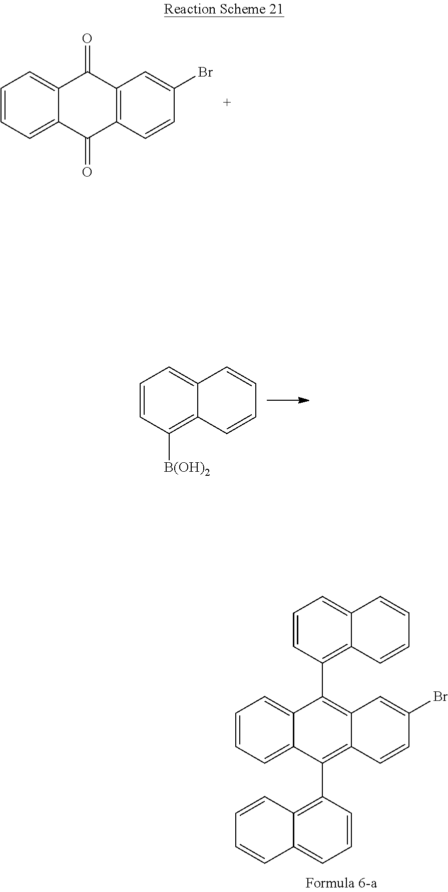

In Reaction Scheme 21, 17.3 g (26.1%) of the compound of Formula 6-a was prepared in the same manner as in Reaction Scheme 3 of Synthesis Example 1, except that 1-naphthalene boronic acid was used instead of 2-bromo-9,9-dimethylfluorene used in Reaction Scheme 3.

b) Synthesis of 6-b

Formula 6-b was synthesized according to Reaction Scheme 22 below.

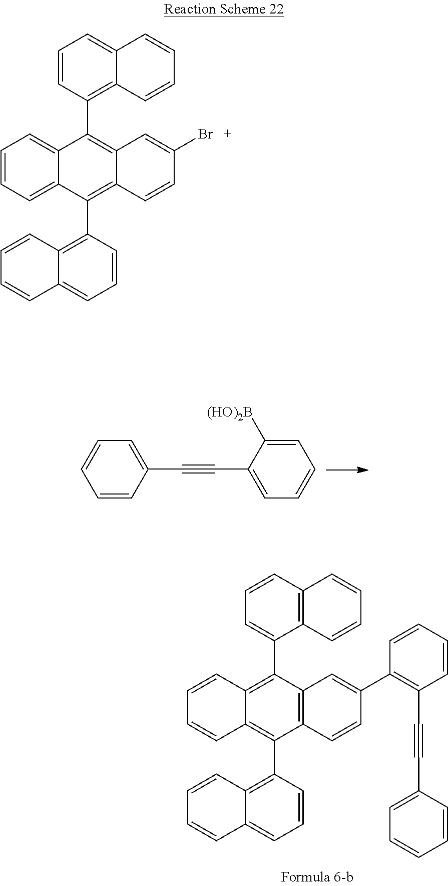

In Reaction Scheme 22, 8.8 g (42.7%) of the compound of Formula 6-b was prepared in the same manner as in Reaction Scheme 4 of Synthesis Example 1, except that the compound of Formula 6-a was used instead of the compound of Formula 1-c used in Reaction Scheme 4.

c) Synthesis of Compound 10

Compound 10 was synthesized according to Reaction Scheme 23 below.

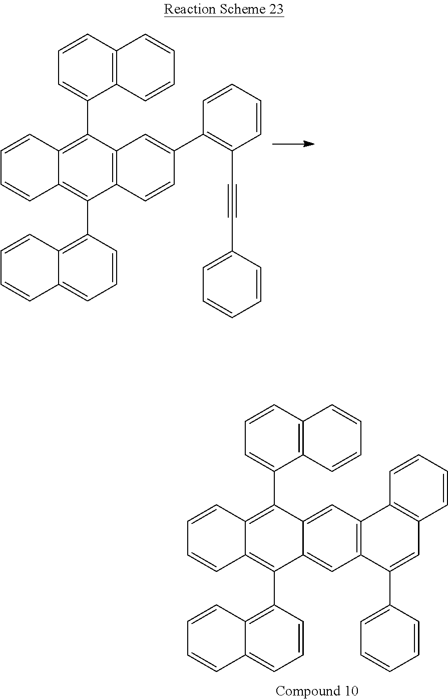

In Reaction Scheme 23, 1.4 g (16.5%) of Compound 10 was prepared in the same manner as in Reaction Scheme 5 of Synthesis Example 1, except that the compound of Formula 6-b was used instead of the compound of Formula 1-d used in Reaction Scheme 5.

MS (MALDI-TOF):m/z=606.23[M]⁺

EA (Elemental Analysis): calc.—C, 95.02%; H, 4.98%.

(i) found—C, 94.99%; H, 5.01%.

¹H NMR (CDCl₃): δ 8.81 (d, 1H, Ar—H), δ 8.34˜8.28 (m, 4H, Ar—H), δ 8.22 (s, 2H, Ar—H), δ 7.94˜7.76 (m, 14H, Ar—H), δ 7.54˜7.21 (m, 9H, Ar—H)

### Synthesis Example 7

### Synthesis of Compound 144

a) Synthesis of 7-a

Formula 7-a was synthesized according to Reaction Scheme 24 below.

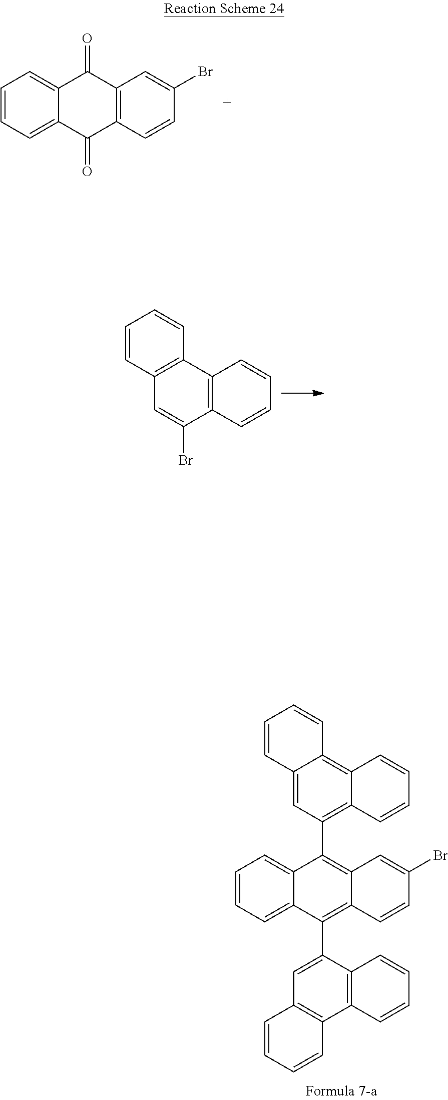

In Reaction Scheme 24, 25.3 g (42.5%) of the compound of Formula 7-a was prepared in the same manner as in Reaction Scheme 3 of Synthesis Example 1, except that 9-bromophenanthrene was used instead of the 2-bromo-9,9-dimethylfluorene used in Reaction Scheme 3.

b) Synthesis of 7-b

Formula 7-b was synthesized according to Reaction Scheme 25 below.

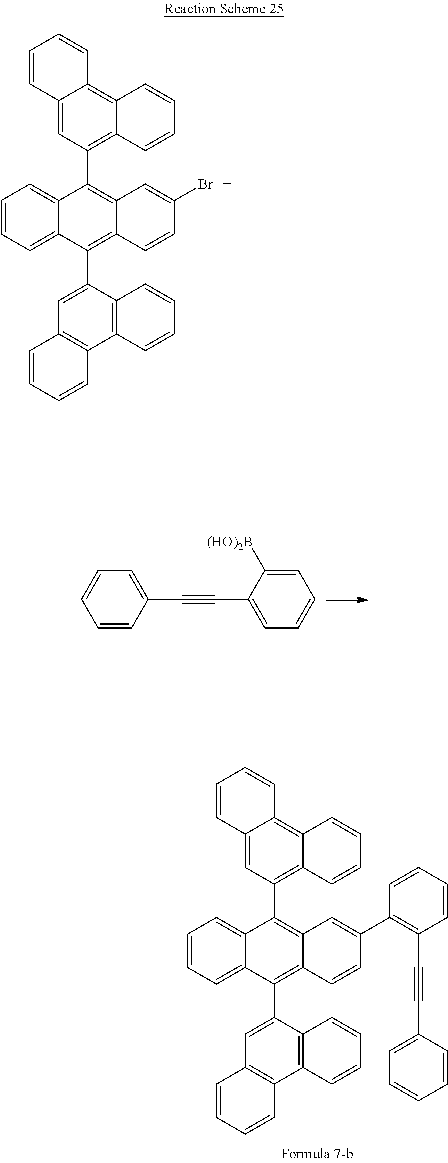

In Reaction Scheme 25, 12.3 g (41.7%) of the compound of Formula 7-b was prepared in the same manner as in Reaction Scheme 4 of Synthesis Example 1, except that the compound of Formula 7-a was used instead of the compound of Formula 1-c used in Reaction Scheme 4.

c) Synthesis of 7-c

Formula 7-c was synthesized according to Reaction Scheme 26 below.

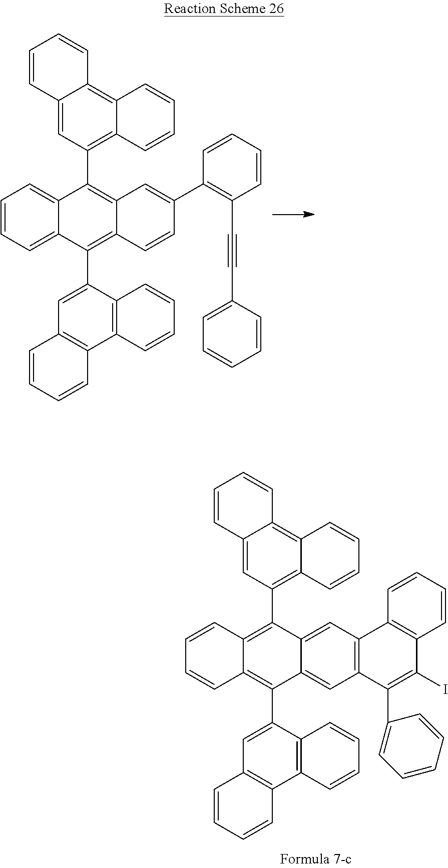

In Reaction Scheme 26, the compound of Formula 7-b was loaded into a 250 ml round bottom flask in a nitrogen atmosphere, and 120 ml of dichloromethane was added thereto. The temperature of the reaction product was decreased to −78° C. and then 34 ml (0.034 mol) of 1.0 M dichloromethane in iodomonochloride was slowly added dropwise thereto.

Stirring was performed thereon for 4 hours and then the reaction was finished. The organic layer was separated using dichloromethane and water, and the residual was concentrated and separated by column chromatography using hexane and methylene chloride as eluents, followed by recrystallization with hexane. The obtained solid was dried to produce 11.5 g (79.3%) of the compound of Formula 7-c.

d) Synthesis of Formula 143

Compound 144 was synthesized according to Reaction Scheme 27 below.

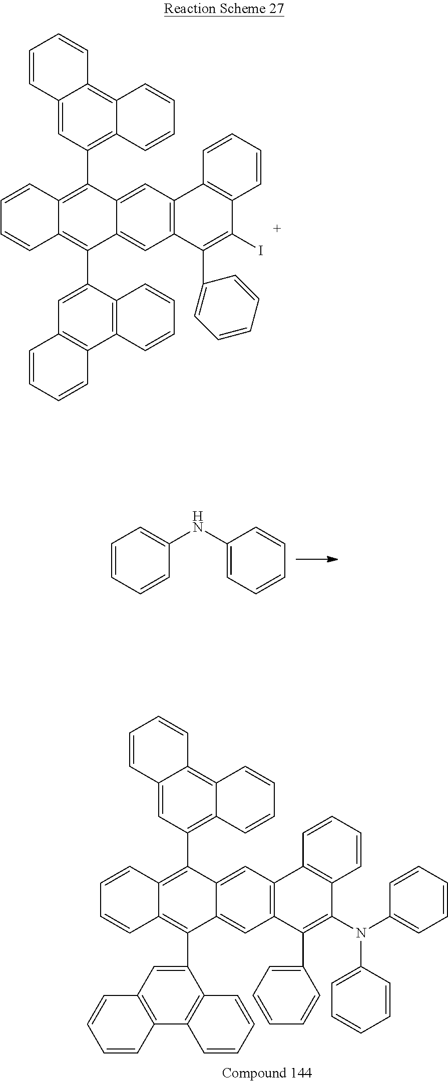

In Reaction Scheme 27, the compound of Formula 7-c, 3.5 g (0.021 mol) of diphenyl amine, 0.062 g (0.00028 mol) of Pd(OAc)₂, 0.17 g (0.00038 mol) of BINAP, and 3.94 g (0.041 mol) of sodium tert-butoxide were loaded into a 500 ml round bottom flask. Then, 300 ml of toluene was added thereto and the mixture was refluxed for 12 hours.

The resulting solution was filtered with hot toluene and the residual was concentrated and separated by column chromatography using hexane and methylene chloride as eluents, followed by recrystallization with hexane to produce 5.3 g (43.3%) of Compound 144.

MS (MALDI-TOF):m/z=873.4[M]⁺

EA (Elemental Analysis): calc.—C, 93.44%; H, 4.96%; N, 1.60%.

(i) found—C, 93.42%; H, 5.01%; N, 1.57%.

¹H NMR (CDCl₃): δ 8.90 (d, 1H, Ar—H), δ 8.24 (s, 2H, Ar—H), δ 8.04˜7.82 (m, 20H, Ar—H), δ 7.64˜7.21 (m, 16H, Ar—H), δ 7.19˜7.08 (m, 4H, Ar—H)

### Synthesis Example 8

### Synthesis of Compound 231

a) Synthesis of Formula 8-a

Formula 8-a was synthesized according to Reaction Scheme 28 below.

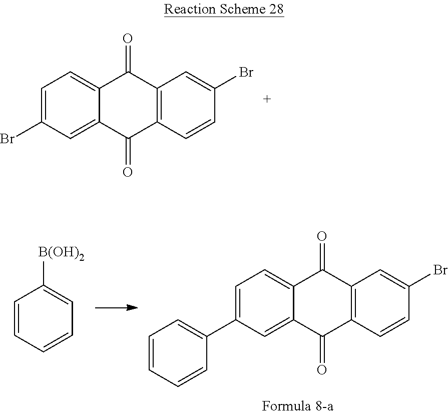

In Reaction Scheme 28, 100 g (0.27 mol) of 2,6-dibromo anthraquinone, 28.32 g (0.23 mol) of a phenyl boronic acid, 6.31 g (0.0055 mol) of Pd(PPh₃)₄, and 75.52 g (0.55 mol) of potassium carbonate were loaded into a 2000 ml round bottom flask. Then, 500 ml of toluene, 500 ml of 1,4-dioxane, and 200 ml of water were added thereto and the mixture was refluxed for 12 hours. The reaction product was cooled and the organic layer was separated using ethylether and water, and concentrated and dried under reduced pressure. The result was separated by column chromatography using hexane and methylene chloride as eluents, followed by recrystallization with hexane. The obtained solid was dried to produce 50.2 g (51.0%) of the compound of Formula 8-a.

b) Synthesis of 8-b

Formula 8-b was synthesized according to Reaction Scheme 29 below.

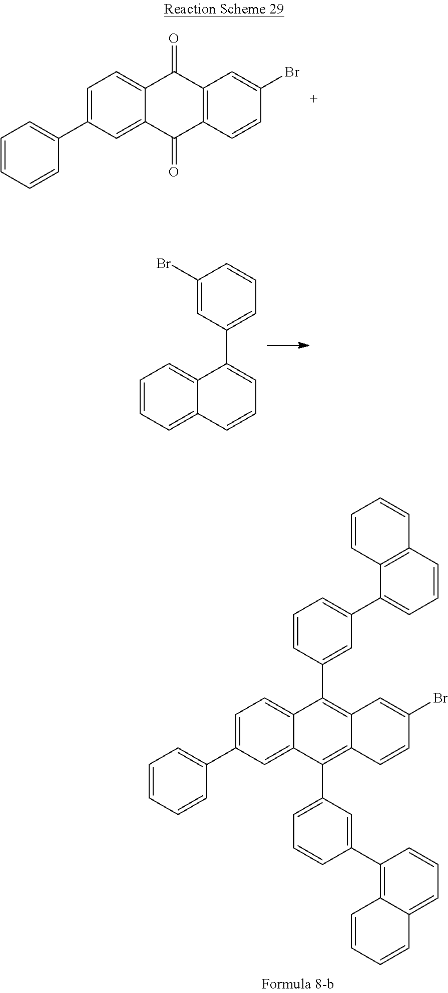

In Reaction Scheme 29, 51.0 g (49.0%) of the compound of Formula 8-b was prepared in the same manner as in Reaction Scheme 3 of Synthesis Example 1, except that 3-bromo phenyl-1-naphthalene was used instead of 2-bromo-9,9-dimethylfluorene used in Reaction Scheme 3.

c) Synthesis of 8-c

Formula 8-c was synthesized according to Reaction Scheme 30 below.

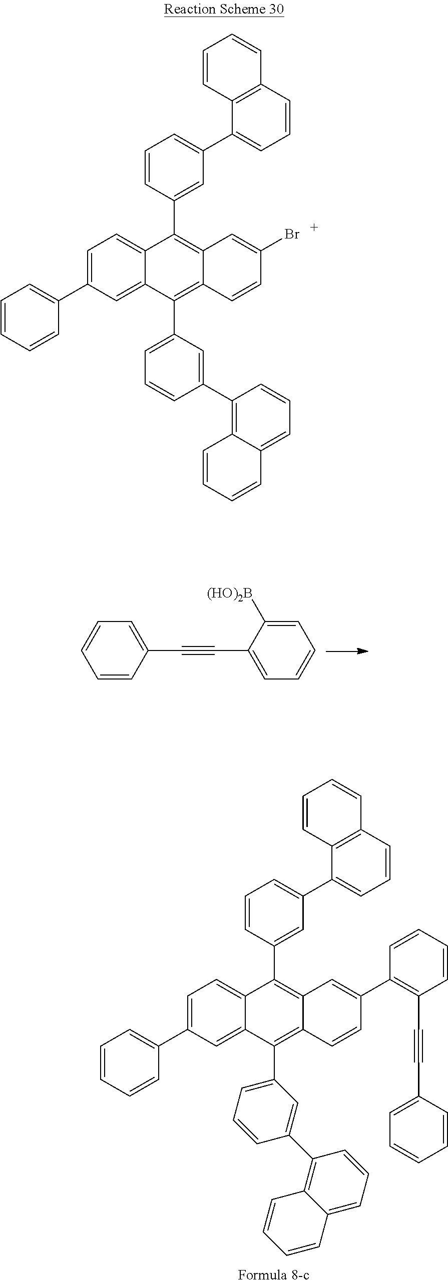

In Reaction Scheme 30, 31.2 g (53.6%) of the compound of Formula 8-c was prepared in the same manner as in Reaction Scheme 4 of Synthesis Example 1, except that the compound of Formula 8-b was used instead of the compound of Formula 1-c used in Reaction Scheme 4.

d) Synthesis of 8-d

Formula 8-d was synthesized according to Reaction Scheme 31 below.

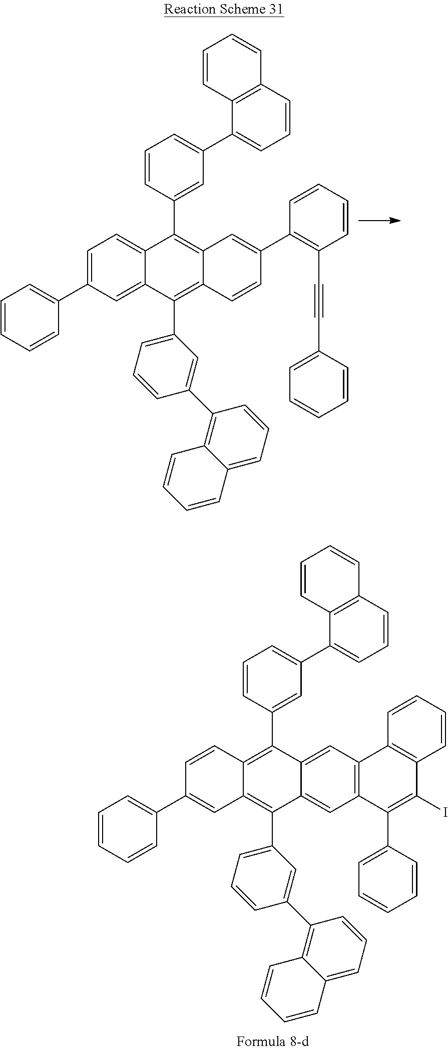

In Reaction Scheme 31, 12.8 g (35.9%) of Formula 8-d was prepared in the same manner as in Reaction Scheme 26 of Synthesis Example 7, except that Formula 8-c was used instead of the Formula 7-b used in Reaction Scheme 26.

Synthesis of Compound 231

Compound 231 was synthesized according to Reaction Scheme 32 below.

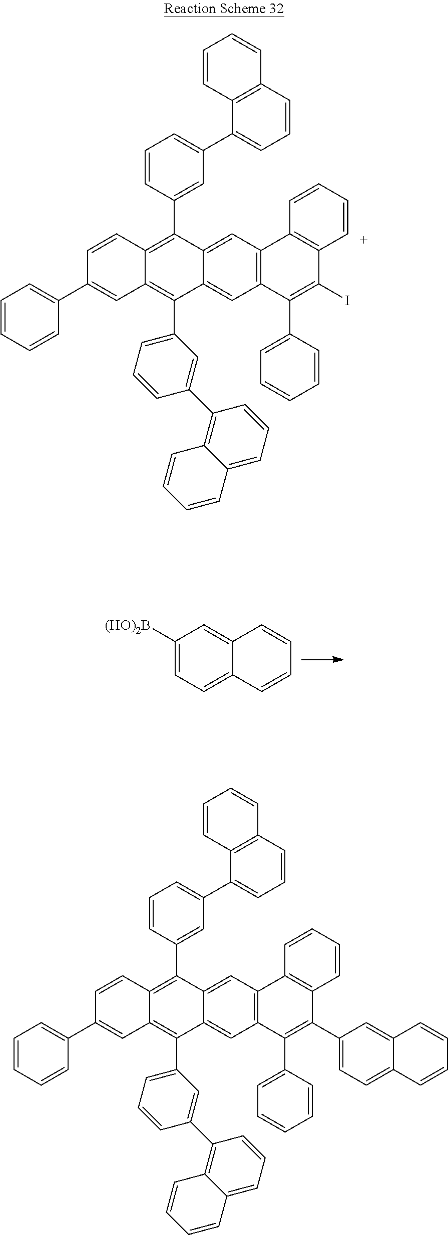

Compound 231

In Reaction Scheme 32, the compound of Formula 8-d obtained from Reaction Scheme 31, 2.92 g (0.017 mol) of 2-naphthyl boronic acid, 0.64 g (0.005 mol) of Pd(pph₃)₄, and 5.52 g (0.040 mol) of potassium carbonate were loaded into a 250 ml round bottom flask, and then 60 ml of toluene, 60 ml of dioxane, and 40 ml of water were added thereto, followed by 12 hours of refluxing. The reaction was finished and the reaction product was subjected to reduced pressure to produce a solid. The organic layer was separated using ethylether and water, concentrated under reduced pressure, and then separated by column chromatography using methylene chloride and hexane as eluents. The obtained solid was dried to produce 2.6 g (66.6%) of Compound 231.

MS (MALDI-TOF):m/z=884.3[M]⁺

EA (Elemental Analysis): calc.—C, 94.99%; H, 5.01%.

(i) found—C, 94.97%; H, 5.03%.

¹H NMR (CDCl₃): δ 8.78 (d, 1H, Ar—H), δ 8.40˜8.34 (m, 3H, Ar—H), δ 8.22 (s, 2H, Ar—H), δ 8.1˜7.7 (m, 14H, Ar—H), δ 7.6˜7.2 (m, 24H, Ar—H)

### Examples

### Manufacture of Organic Light-Emitting Diode

ITO glass was patterned to have a light emission area of 2 mm×2 mm and then washed. The substrate was installed in a vacuum chamber, and then, at a base pressure of 1×10⁻⁶ torr, an organic material was deposited on the ITO glass in the sequence of DNTPD (700 Å); NPD (300 Å); Compounds 10, 24, 28, 29, 83, 144, 231, or 330; RD (1.0%) (400 Å); Alq3 (300 Å); LiF (5 Å); and Al (1,000 Å). The formed organic light-emitting diode was measured at 0.4 mA. The structure of RD is illustrated below:

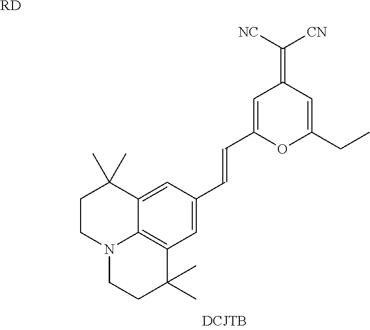

### Comparative Example

An organic light-emitting diode was manufactured in the same manner as in the above Examples, except that rubrene and compound 500 were used instead of the compound according to embodiments of the present invention.

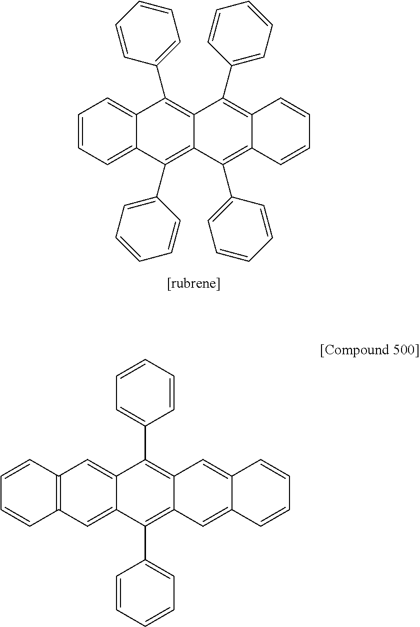

TABLE 1

| | | | | | EL | | |
|---|---|---|---|---|---|---|---|
| | | | Doping | | Eff. | | |
| | | | Concen- | | (cd/ | | |
| | Formula | Dopant | tration % | V | A) | CIEx | CIEy |
| Example 1 | 28 | RD | 1.0% | 3.8 | 6.33 | 0.63 | 0.36 |
| Example 2 | 24 | | | 4.3 | 6.33 | 0.65 | 0.35 |
| Example 3 | 83 | | | 4.1 | 7.78 | 0.63 | 0.36 |
| Example 4 | 330 | | | 3.9 | 10.01 | 0.64 | 0.36 |
| Example 5 | 29 | | | 3.7 | 8.01 | 0.64 | 0.36 |
| Example 6 | 10 | | | 3.8 | 11.2 | 0.65 | 0.36 |
| Example 7 | 144 | | | 4.1 | 12.5 | 0.63 | 0.35 |
| Example 8 | 231 | | | 3.9 | 11.8 | 0.65 | 0.36 |
| Comparative | rubrene | | | 4.5 | 2.98 | 0.65 | 0.34 |
| Example 1 | | | | | | | |
| Comparative | compound | | | 4.3 | 5.76 | 0.64 | 0.36 |
| Example 2 | 500 | | | | | | |

As shown in Table 1, when the compounds according to embodiments of the present invention are used as a host material, the OLEDs show lower driving voltage and higher luminescent efficiency than when rubrene and compound 500, which are commonly used, are used as the host material.

The novel compounds according to embodiments of the present invention are more stable and have better luminescent characteristics than conventional materials. Thus, organic light-emitting devices including the compounds have improved luminescent efficiency and low driving voltage.

While the present invention has been illustrated and described with reference to certain exemplary embodiments thereof, it will be understood by those of ordinary skill in the art that various changes and modifications may be made to the described embodiments without departing from the spirit and scope of the present invention as defined by the following claims.

What is claimed is:

1. A compound comprising a compound represented by Formula 1: 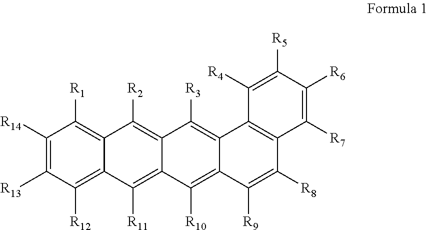
wherein:
each of R₁ to R₁₄ is independently a hydrogen atom, a deuterium atom, a substituted or unsubstituted C1 to C60 alkyl group, a substituted or unsubstituted C2 to C60 alkenyl group, a substituted or unsubstituted C2 to C60 alkynyl group, a substituted or unsubstituted C3 to C60 cycloalkyl group, a substituted or unsubstituted C1 to C60 alkoxy group, a substituted or unsubstituted C5 to C60 aryloxy group, a substituted or unsubstituted C5 to C60 arylthio group, a substituted or unsubstituted C5 to C60 aryl group, an amino group substituted with a C5 to C60 aryl group or a C3 to C60 heteroaryl group, a substituted or unsubstituted C3 to C60 heteroaryl group, a substituted or unsubstituted C6 to C60 fused polycyclic ring, a halogen atom, a cyano group, a nitro group, a hydroxyl group, or a carboxyl group.

2. The compound of claim 1, wherein each of R₁ to R₁₄ is independently a hydrogen atom, a deuterium atom, a cyano group, a halogen atom, a substituted or unsubstituted C1 to C30 alkyl group, a substituted or unsubstituted C5 to C30 aryl group, a substituted or unsubstituted C3 to C30 heteroaryl group, an amino group substituted with a C5 to C30 aryl group or a C3 to C30 heteroaryl group, or a substituted or unsubstituted C6 to C30 fused polycyclic ring.

3. The compound of claim 1, wherein each of R₁ to R₁₄ is independently a hydrogen atom, a deuterium atom, a halogen atom, a cyano group, a substituted or unsubstituted C1 to C20 alkyl group, or a compound represented by one of Formulae 2a to 2g: 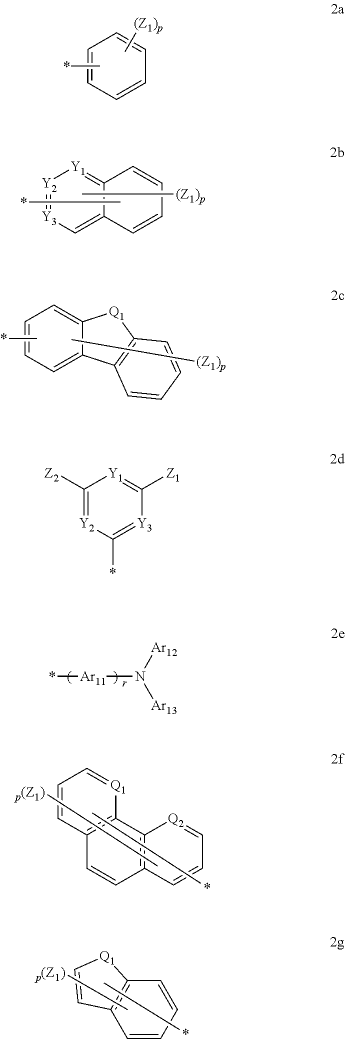
wherein:
each of Q₁ and Q₂ is independently a linker represented by —C(R₂₀)(R₂₁)—, —N(R₂₀)—, —S—, or —O—;
each of Y₁, Y₂, and Y₃ is independently a linker represented by —N═ or —C(R₂₂)═;
each of Z₁, Z₂, Ar₁₂, Ar₁₃, R₂₀, R₂₁, and R₂₂ is independently a hydrogen atom, a deuterium atom, substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group, wherein adjacent Ar₁₂ and Ar₁₃ groups or adjacent R₂₀ and R₂₁ groups are optionally fused with each other to form a ring or are optionally linked to each other via a single bond;
Ar₁₁ is a substituted or unsubstituted C1 to C20 alkylene group, a substituted or unsubstituted C5 to C20 arylene group, or a substituted or unsubstituted C3 to C20 heteroarylene group;
p is an integer of 1 to 12;
r is an integer of 0 to 5; and
* is a binding site.

4. The compound of claim 1, wherein each of R₁, R₃, R₄, R₇, R₁₀, and R₁₂ to R₁₄ is independently a hydrogen atom or a deuterium atom.

5. The compound of claim 1, wherein R₂ and R₁₁ are identical to each other.

6. The compound of claim 1, wherein each of R₁ to R₁₄ is independently a hydrogen atom, a deuterium atom, a halogen atom, a cyano group, a substituted or unsubstituted C1 to C20 alkyl group, a substituted or unsubstituted C3 to C30 heteroaryl group, a substituted or unsubstituted C6 to C30 fused polycyclic ring, or a compound represented by one of Formula 3a to 3f: 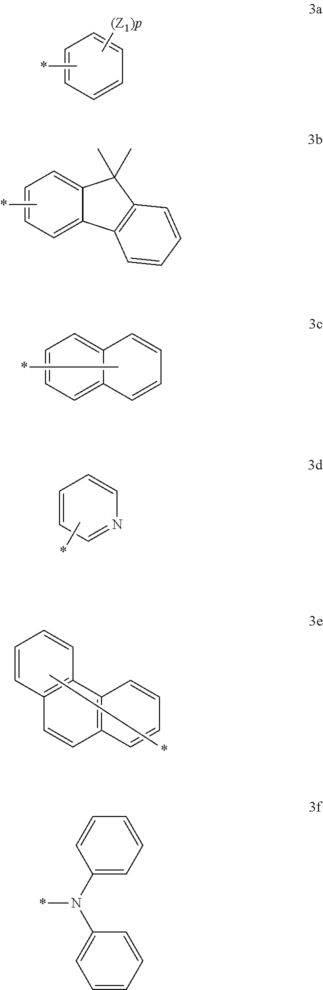
wherein:
Z₁ is a hydrogen atom, a deuterium atom, or a substituted or unsubstituted C5 to C20 aryl group;
p is an integer of 1 to 5; and
* is a binding site.

7. The compound of claim 1, wherein each of R₁, R₃, R₄, R₇, R₁₀, and R₁₂ to R₁₄ is independently a hydrogen atom or a deuterium atom, and each of R₂, R₅, R₆, R₈, R₉, and R₁₁ is independently a hydrogen atom, a deuterium atom, a halogen atom, a cyano group, a substituted or unsubstituted C1 to C20 alkyl group, or a compound represented by one of Formulae 2a to 2g: 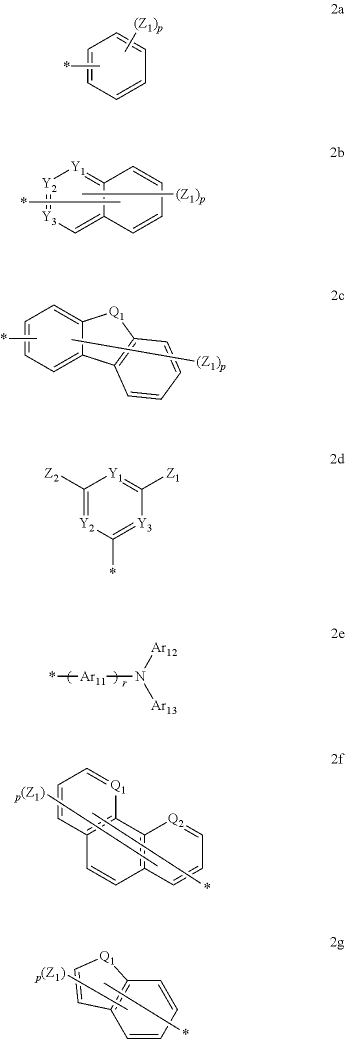
wherein:
each of Q₁ and Q₂ is independently a linker represented by —C(R₂₀)(R₂₁)—, —N(R₂₀)—, —S—, or —O—;
each of Y₁, Y₂, and Y₃ is independently a linker represented by —N═ or —C(R₂₂)═;
each of Z₁, Z₂, Ar₁₂, Ar₁₃, R₂₀, R₂₁, and R₂₂ is independently a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group, wherein adjacent Ar₁₂ and Ar₁₃ groups, or adjacent R₂₀ and R₂₁ groups are optionally fused with each other to form a ring, or are optionally linked to each other via a single bond;
Ar₁₁ is a substituted or unsubstituted C1 to C20 alkylene group, a substituted or unsubstituted C5 to C20 arylene group, or a substituted or unsubstituted C3 to C20 heteroarylene group;
p is an integer of 1 to 12;
r is an integer of 0 to 5; and
* is a binding site.

8. The compound of claim 1, wherein each of R₁, R₃, R₄, R₇, R₁₀, and R₁₂ to R₁₄ is independently a hydrogen atom or a deuterium atom, and each of R₂, R₅, R₆, R₈, R₉, and R₁₁ is independently a hydrogen atom, a deuterium atom, a halogen atom, a cyano group, a substituted or unsubstituted C1 to C20 alkyl group, or a compound represented by one of Formula 3a to 3f: 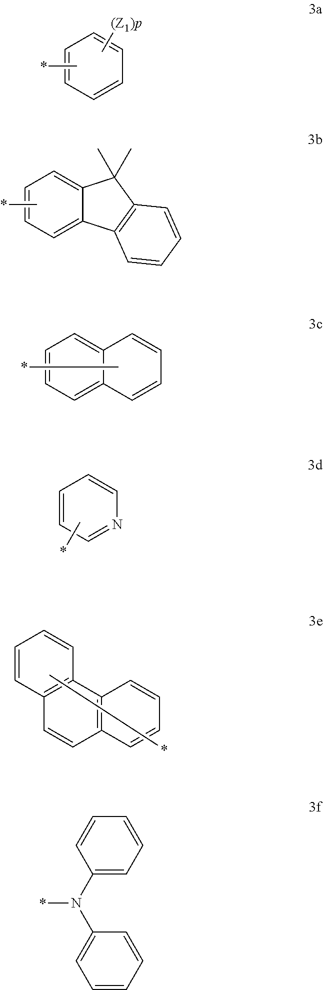
wherein:
Z₁ is a hydrogen atom, a deuterium atom, or a substituted or unsubstituted C5 to C20 aryl group;
p is an integer of 1 to 5; and
* is a binding site.

9. The compound of claim 1, wherein each of R₁, R₃, R₄, R₇, R₁₀, and R₁₂ to R₁₄ is independently a hydrogen atom or a deuterium atom, and each of R₂, R₅, R₆, R₈, R₉, and R₁₁ is independently a hydrogen atom, a deuterium atom, a halogen atom, a cyano group, a substituted or unsubstituted C1 to C20 alkyl group, or a compound represented by one of Formulae 2a to 2g, and R₂ and R₁₁ are identical to each other: 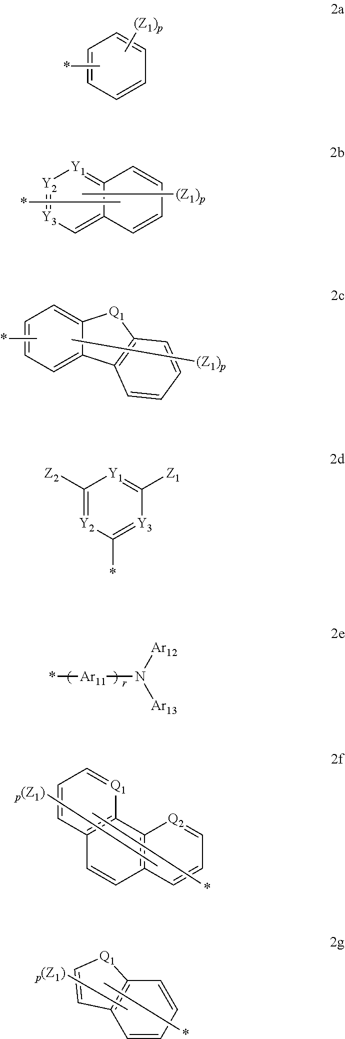
wherein:
each of Q₁ and Q₂ is independently a linker represented by —C(R₂₀)(R₂₁)—, —N(R₂₀)—, —S—, or —O—;
each of Y₁, Y₂, and Y₃ is independently a linker represented by —N═, or —C(R₂₂)═;
each of Z₁, Z₂, Ar₁₂, Ar₁₃, R₂₀, R₂₁, and R₂₂ is independently a hydrogen atom, a deuterium atom, a substituted or unsubstituted C1 to C20 alkyl group, a substituted or unsubstituted C5 to C20 aryl group, a substituted or unsubstituted C3 to C20 heteroaryl group, a substituted or unsubstituted C6 to C20 fused polycyclic ring, a halogen atom, a cyano group, a nitro group, a hydroxyl group, a carboxyl group, or a substituted silyl group, wherein adjacent Ar₁₂ and Ar₁₃ groups or adjacent R₂₀ and R₂₁ groups are optionally fused with each other to form ring, or optionally linked to each other via a single bond;
Ar₁₁ is a substituted or unsubstituted C1 to C20 alkylene group, a substituted or unsubstituted C5 to C20 arylene group, or a substituted or unsubstituted C3 to C20 heteroarylene group;
p is an integer of 1 to 12;
r is an integer of 0 to 5; and
* is a binding site.

10. The compound of claim 1, wherein the compound of Formula 1 is one of Compounds 10, 24, 28, 29, 83, 144, 231 or 330:

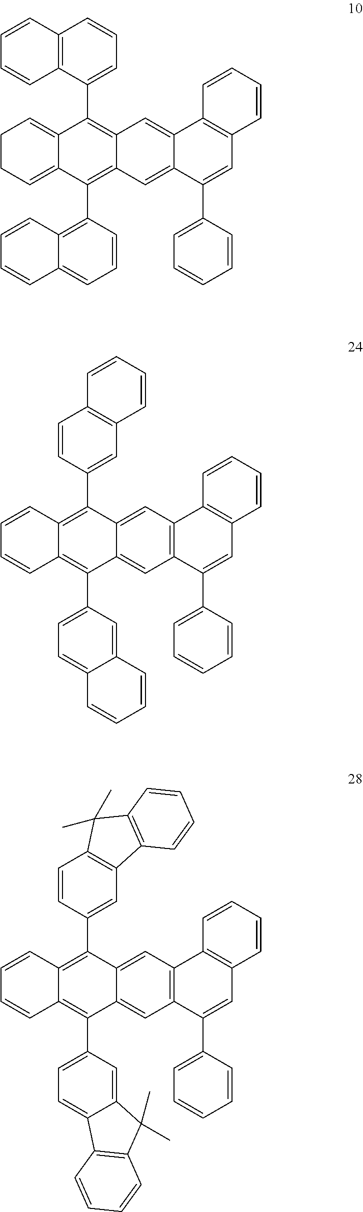

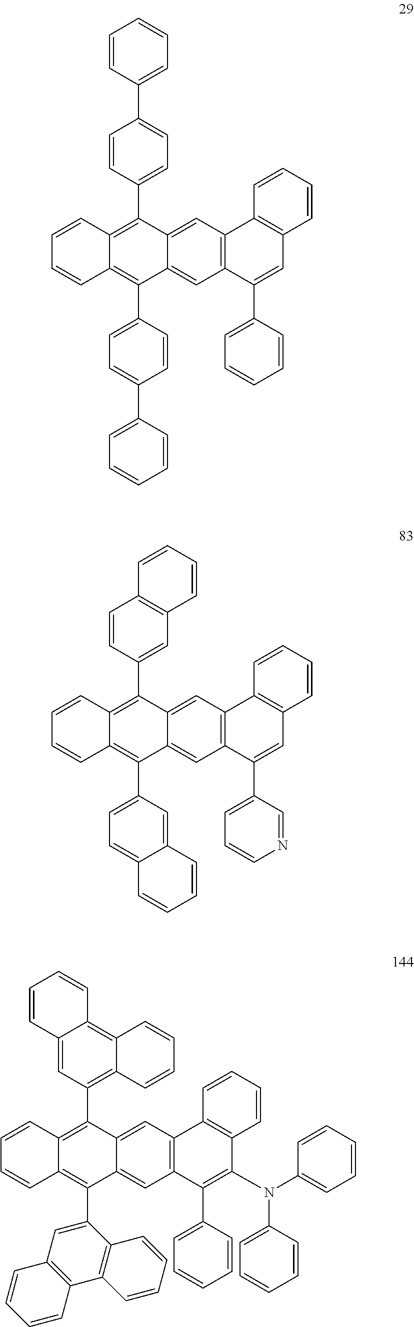

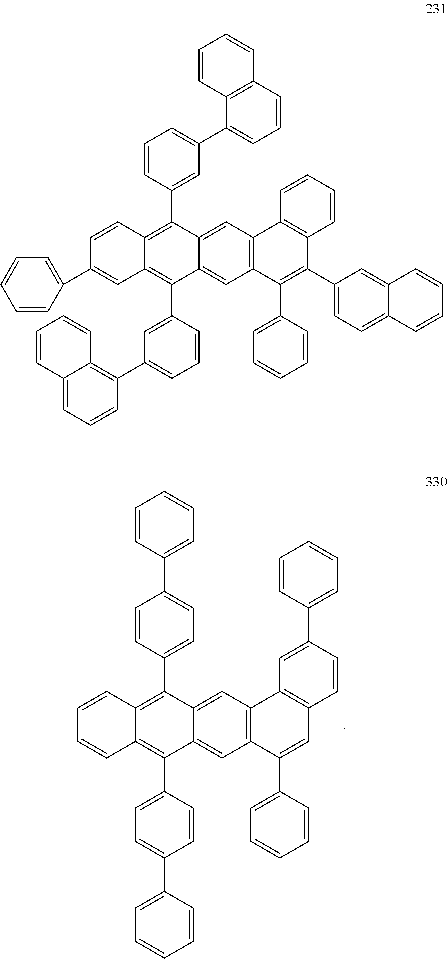

11. An organic light-emitting device comprising:
a first electrode;
a second electrode; and
an organic layer between the first electrode and the second electrode,
wherein the organic layer comprises the compound of claim 1.

12. The organic light-emitting device of claim 11, wherein the organic layer is a hole injection layer, a hole transport layer, a functional layer having hole injection and hole transport capabilities, an electron injection layer, an electron transport layer, or a functional layer having electron transport and electron injection capabilities.

13. The organic light-emitting device of claim 11, wherein the organic light-emitting device comprises an electron transport layer that comprises an electron transporting organic material and a metal-containing material.

14. The organic light-emitting device of claim 11, wherein the organic layer comprises an emission layer and the compound represented by Formula 1 is used as a host for a fluorescence or phosphorescence device.

15. The organic light-emitting device of claim 11, wherein the organic light-emitting device comprises an emission layer, a hole transport layer, and an electron transport layer,
wherein the emission layer further comprises an anthracene compound, an arylamine compound, or a styryl compound.

16. The organic light-emitting device of claim 11, wherein the organic light-emitting device comprises an emission layer, a hole transport layer, or an electron transport layer,
wherein the emission layer comprises a red layer, a green layer, a blue layer, and a white layer and one of the red, green, blue or white layers further comprises a phosphorescent compound.

17. The organic light-emitting device of claim 11, wherein the organic layer is a red emission layer.

18. The organic light-emitting device of claim 11, wherein the organic layer is a red emission layer and the compound of Formula 1 is used as a red host.

19. The organic light-emitting device of claim 11, wherein the organic layer is formed using the compound of claim 1 in a wet process.

20. A flat panel display device comprising the organic light-emitting device of claim 11,
wherein the first electrode of the organic light-emitting device is electrically connected to a source electrode or a drain electrode of a thin film transistor.
